SEQUENCE LISTING (68 sequences)

SEQ ID NO: 1 - DNA, 22 bp, synthetic construct
  misc_feature 1..22: note = artificial sequence for RNAi
  misc_feature 8..9: note = n is a, c, g, or t
  misc_feature 22: note = n is a, c, g, or t
  source 1..22: mol_type = other DNA
ttcgwwwnnahhwwcatccggn

SEQ ID NO: 2 - RNA, 22 nt, synthetic construct
  misc_feature 1..22: note = artificial sequence for RNAi
  misc_feature 8..9: note = n is a, c, g, or u
  misc_feature 22: note = n is a, c, g, or u
  source 1..22: mol_type = other RNA
ttcgwwwnnahhwwcatccggn

SEQ ID NO: 3 - DNA, 22 bp, synthetic construct
  misc_feature 1..22: note = artificial sequence for RNAi
  source 1..22: mol_type = other DNA
ttcgaattaaacttcatccgga

SEQ ID NO: 4 - RNA, 22 nt, synthetic construct
  misc_feature 1..22: note = artificial sequence for RNAi
  source 1..22: mol_type = other RNA
ttcgaattaaacttcatccgga

SEQ ID NO: 5 - DNA, 22 bp, synthetic construct
  misc_feature 1..22: note = artificial sequence for RNAi
  source 1..22: mol_type = other DNA
ttcgataacaatatcatccgga

SEQ ID NO: 6 - RNA, 22 nt, synthetic construct
  misc_feature 1..22: note = artificial sequence for RNAi
  source 1..22: mol_type = other RNA
ttcgataacaatatcatccgga

SEQ ID NO: 7 - DNA, 22 bp, synthetic construct
  misc_feature 1..22: note = artificial sequence for RNAi
  source 1..22: mol_type = other DNA
ttcgatataaacttcatccgga

SEQ ID NO: 8 - RNA, 22 nt, synthetic construct
  misc_feature 1..22: note = artificial sequence for RNAi
  source 1..22: mol_type = other RNA
ttcgatataaacttcatccgga

SEQ ID NO: 9 - DNA, 22 bp, synthetic construct
  misc_feature 1..22: note = artificial sequence for RNAi
  source 1..22: mol_type = other DNA
ttcgattaaaacatcatccgga

SEQ ID NO: 10 - RNA, 22 nt, synthetic construct
  misc_feature 1..22: note = artificial sequence for RNAi
  source 1..22: mol_type = other RNA
ttcgattaaaacatcatccgga

SEQ ID NO: 11 - DNA, 22 bp, synthetic construct
  misc_feature 1..22: note = artificial sequence for RNAi
  source 1..22: mol_type = other DNA
ttcgtatacaatatcatccgga

SEQ ID NO: 12 - RNA, 22 nt, synthetic construct
  misc_feature 1..22: note = artificial sequence for RNAi
  source 1..22: mol_type = other RNA
ttcgtatacaatatcatccgga

SEQ ID NO: 13 - DNA, 22 bp, synthetic construct
  misc_feature 1..22: note = artificial sequence for RNAi
  source 1..22: mol_type = other DNA
ttcgtatgcaatatcatccgga

SEQ ID NO: 14 - RNA, 22 nt, synthetic construct
  misc_feature 1..22: note = artificial sequence for RNAi
  source 1..22: mol_type = other RNA
ttcgtatgcaatatcatccgga

SEQ ID NO: 15 - DNA, 97 bp, synthetic construct
  misc_feature 1..97: note = artificial sequence for RNAi
  source 1..97: mol_type = other DNA
tgctgttgacagtgagcgcccggatgaagtttaattcgaatagtgaagccacagatgtattcgaattaaacttcatccggatgcctactgcctcgga

SEQ ID NO: 16 - RNA, 97 nt, synthetic construct
  misc_feature 1..97: note = artificial sequence for RNAi
  source 1..97: mol_type = other RNA
tgctgttgacagtgagcgcccggatgaagtttaattcgaatagtgaagccacagatgtattcgaattaaacttcatccggatgcctactgcctcgga

SEQ ID NO: 17 - DNA, 97 bp, synthetic construct
  misc_feature 1..97: note = artificial sequence for RNAi
  source 1..97: mol_type = other DNA
tgctgttgacagtgagcgcccggatgatattgttatcgaatagtgaagccacagatgtattcgataacaatatcatccggatgcctactgcctcgga

SEQ ID NO: 18 - RNA, 97 nt, synthetic construct
  misc_feature 1..97: note = artificial sequence for RNAi
  source 1..97: mol_type = other RNA
tgctgttgacagtgagcgcccggatgatattgttatcgaatagtgaagccacagatgtattcgataacaatatcatccggatgcctactgcctcgga

SEQ ID NO: 19 - DNA, 97 bp, synthetic construct
  misc_feature 1..97: note = artificial sequence for RNAi
  source 1..97: mol_type = other DNA
tgctgttgacagtgagcgcccggatgaagtttatatcgaatagtgaagccacagatgtattcgatataaacttcatccggatgcctactgcctcgga

SEQ ID NO: 20 - RNA, 97 nt, synthetic construct
  misc_feature 1..97: note = artificial sequence for RNAi
  source 1..97: mol_type = other RNA
tgctgttgacagtgagcgcccggatgaagtttatatcgaatagtgaagccacagatgtattcgatataaacttcatccggatgcctactgcctcgga

SEQ ID NO: 21 - DNA, 97 bp, synthetic construct
  misc_feature 1..97: note = artificial sequence for RNAi
  source 1..97: mol_type = other DNA
tgctgttgacagtgagcgcccggatgatgttttaatcgaatagtgaagccacagatgtattcgattaaaacatcatccggatgcctactgcctcgga

SEQ ID NO: 22 - RNA, 97 nt, synthetic construct
  misc_feature 1..97: note = artificial sequence for RNAi
  source 1..97: mol_type = other RNA
tgctgttgacagtgagcgcccggatgatgttttaatcgaatagtgaagccacagatgtattcgattaaaacatcatccggatgcctactgcctcgga

SEQ ID NO: 23 - DNA, 97 bp, synthetic construct
  misc_feature 1..97: note = artificial sequence for RNAi
  source 1..97: mol_type = other DNA
tgctgttgacagtgagcgcccggatgatattgtatacgaatagtgaagccacagatgtattcgtatacaatatcatccggatgcctactgcctcgga

SEQ ID NO: 24 - RNA, 97 nt, synthetic construct
  misc_feature 1..97: note = artificial sequence for RNAi
  source 1..97: mol_type = other RNA
tgctgttgacagtgagcgcccggatgatattgtatacgaatagtgaagccacagatgtattcgtatacaatatcatccggatgcctactgcctcgga

SEQ ID NO: 25 - DNA, 97 bp, synthetic construct
  misc_feature 1..97: note = artificial sequence for RNAi
  source 1..97: mol_type = other DNA
tgctgttgacagtgagcgcccggatgatattgcatacgaatagtgaagccacagatgtattcgtatgcaatatcatccggatgcctactgcctcgga

SEQ ID NO: 26 - RNA, 97 nt, synthetic construct
  misc_feature 1..97: note = artificial sequence for RNAi
  source 1..97: mol_type = other RNA
tgctgttgacagtgagcgcccggatgatattgcatacgaatagtgaagccacagatgtattcgtatgcaatatcatccggatgcctactgcctcgga

SEQ ID NO: 27 - DNA, 22 bp, synthetic construct
  misc_feature 1..22: note = artificial sequence for RNAi
  misc_feature 1: note = n is a, c, g, or t
  misc_feature 14..15: note = n is a, c, g, or t
  source 1..22: mol_type = other DNA
nccggatgwwddtnnwwwcgaa

SEQ ID NO: 28 - RNA, 22 nt, synthetic construct
  misc_feature 1..22: note = artificial sequence for RNAi
  misc_feature 1: note = n is a, c, g, or u
  misc_feature 14..15: note = n is a, c, g, or u
  source 1..22: mol_type = other RNA
nccggatgwwddtnnwwwcgaa

SEQ ID NO: 29 - DNA, 22 bp, synthetic construct
  misc_feature 1..22: note = artificial sequence for RNAi
  source 1..22: mol_type = other DNA
tccggatgaagtttaattcgaa

SEQ ID NO: 30 - RNA, 22 nt, synthetic construct
  misc_feature 1..22: note = artificial sequence for RNAi
  source 1..22: mol_type = other RNA
tccggatgaagtttaattcgaa

SEQ ID NO: 31 - DNA, 22 bp, synthetic construct
  misc_feature 1..22: note = artificial sequence for RNAi
  source 1..22: mol_type = other DNA
tccggatgatattgttatcgaa

SEQ ID NO: 32 - RNA, 22 nt, synthetic construct
  misc_feature 1..22: note = artificial sequence for RNAi
  source 1..22: mol_type = other RNA
tccggatgatattgttatcgaa

SEQ ID NO: 33 - DNA, 22 bp, synthetic construct
  misc_feature 1..22: note = artificial sequence for RNAi
  source 1..22: mol_type = other DNA
tccggatgaagtttatatcgaa

SEQ ID NO: 34 - RNA, 22 nt, synthetic construct
  misc_feature 1..22: note = artificial sequence for RNAi
  source 1..22: mol_type = other RNA
tccggatgaagtttatatcgaa

SEQ ID NO: 35 - DNA, 22 bp, synthetic construct
  misc_feature 1..22: note = artificial sequence for RNAi
  source 1..22: mol_type = other DNA
tccggatgatgttttaatcgaa

SEQ ID NO: 36 - RNA, 22 nt, synthetic construct
  misc_feature 1..22: note = artificial sequence for RNAi
  source 1..22: mol_type = other RNA
tccggatgatgttttaatcgaa

SEQ ID NO: 37 - DNA, 22 bp, synthetic construct
  misc_feature 1..22: note = artificial sequence for RNAi
  source 1..22: mol_type = other DNA
tccggatgatattgtatacgaa

SEQ ID NO: 38 - RNA, 22 nt, synthetic construct
  misc_feature 1..22: note = artificial sequence for RNAi
  source 1..22: mol_type = other RNA
tccggatgatattgtatacgaa

SEQ ID NO: 39 - DNA, 22 bp, synthetic construct
  misc_feature 1..22: note = artificial sequence for RNAi
  source 1..22: mol_type = other DNA
tccggatgatattgcatacgaa

SEQ ID NO: 40 - RNA, 22 nt, synthetic construct
  misc_feature 1..22: note = artificial sequence for RNAi
  source 1..22: mol_type = other RNA
tccggatgatattgcatacgaa

SEQ ID NO: 41 - DNA, 169 bp, synthetic construct
  misc_feature 1..169: note = artificial sequence for RNAi
  misc_feature 148..169: note = n is a, c, g, or t
  source 1..169: mol_type = other DNA
taggaattataatgcttatctacgtttgaccgcctgaagtctctgattaagtatctgccagctaaaggtgaagatatattccccatccactacaagtacatgtgtaatagctttgcacaggaattttgtttaatataactcgagcgannnnnnnnnnnnnnnnnnnnnn

SEQ ID NO: 42 - RNA, 169 nt, synthetic construct
  misc_feature 1..169: note = artificial sequence for RNAi
  misc_feature 148..169: note = n is a, c, g, or u
  source 1..169: mol_type = other RNA
taggaattataatgcttatctacgtttgaccgcctgaagtctctgattaagtatctgccagctaaaggtgaagatatattccccatccactacaagtacatgtgtaatagctttgcacaggaattttgtttaatataactcgagcgannnnnnnnnnnnnnnnnnnnnn

SEQ ID NO: 43 - DNA, 169 bp, synthetic construct; identical to SEQ ID NO: 41
  misc_feature 1..169: note = artificial sequence for RNAi
  misc_feature 148..169: note = n is a, c, g, or t
  source 1..169: mol_type = other DNA

SEQ ID NO: 44 - RNA, 169 nt, synthetic construct; identical to SEQ ID NO: 42
  misc_feature 1..169: note = artificial sequence for RNAi
  misc_feature 148..169: note = n is a, c, g, or u
  source 1..169: mol_type = other RNA

SEQ ID NO: 45 - DNA, 169 bp, synthetic construct; identical to SEQ ID NO: 41
  misc_feature 1..169: note = artificial sequence for RNAi
  misc_feature 148..169: note = n is a, c, g, or t
  source 1..169: mol_type = other DNA

SEQ ID NO: 46 - RNA, 169 nt, synthetic construct; identical to SEQ ID NO: 42
  misc_feature 1..169: note = artificial sequence for RNAi
  misc_feature 148..169: note = n is a, c, g, or u
  source 1..169: mol_type = other RNA

SEQ ID NO: 47 - DNA, 169 bp, synthetic construct; identical to SEQ ID NO: 41
  misc_feature 1..169: note = artificial sequence for RNAi
  misc_feature 148..169: note = n is a, c, g, or t
  source 1..169: mol_type = other DNA

SEQ ID NO: 48 - RNA, 169 nt, synthetic construct; identical to SEQ ID NO: 42
  misc_feature 1..169: note = artificial sequence for RNAi
  misc_feature 148..169: note = n is a, c, g, or u
  source 1..169: mol_type = other RNA

SEQ ID NO: 49 - DNA, 169 bp, synthetic construct; identical to SEQ ID NO: 41
  misc_feature 1..169: note = artificial sequence for RNAi
  misc_feature 148..169: note = n is a, c, g, or t
  source 1..169: mol_type = other DNA

SEQ ID NO: 50 - RNA, 169 nt, synthetic construct; identical to SEQ ID NO: 42
  misc_feature 1..169: note = artificial sequence for RNAi
  misc_feature 148..169: note = n is a, c, g, or u
  source 1..169: mol_type = other RNA

SEQ ID NO: 51 - DNA, 169 bp, synthetic construct; identical to SEQ ID NO: 41
  misc_feature 1..169: note = artificial sequence for RNAi
  misc_feature 148..169: note = n is a, c, g, or t
  source 1..169: mol_type = other DNA

SEQ ID NO: 52 - RNA, 169 nt, synthetic construct; identical to SEQ ID NO: 42
  misc_feature 1..169: note = artificial sequence for RNAi
  misc_feature 148..169: note = n is a, c, g, or u
  source 1..169: mol_type = other RNA

SEQ ID NO: 53 - DNA, 4425 bp, synthetic construct
  misc_feature 1..4425: note = artificial sequence for RNAi
  source 1..4425: mol_type = other DNA
atgcgaccaagcggaaccgctggagccgctctgctggctctgctggccgccctgtgccctgccagcagggctctggaggaaaagaaagtgtgccaggggacatccaacaagctgacacagctgggaactttcgaggatcactttctgagcctgcagagaatgtttaacaattgcgaagtggtgctgggcaacctggagatcacttacgtgcagaggaattatgacctgagcttcctgaagaccattcaggaagtggccggatacgtgctgatcgctctgaacacagtggaacggatccccctggagaatctgcagatcattaggggcaacatgtactatgagaacagctacgccctggctgtgctgagcaactatgacgccaataagaccgggctgaaagaactgccaatgagaaatctgcaggagatcctgcatggagccgtgaggttctcaaacaatcccgccctgtgcaacgtggagagcatccagtggagggacattgtgagctccgatttcctgagcaacatgtccatggattttcagaatcacctggggtcctgccagaagtgtgacccaagttgtcccaacggatcatgctggggagccggcgaggaaaattgccagaagctgactaaaatcatttgcgctcagcagtgttccggccgctgcagggggaaatctcctagtgattgctgtcataatcagtgtgctgctggatgcaccggaccacgcgaatctgactgcctggtgtgccgcaagtttcgggacgaggccacctgtaaagatacatgcccccctctgatgctgtacaaccccaccacatatcagatggatgtgaatcctgaggggaagtactccttcggagccacctgcgtgaagaagtgccctcgcaactatgtggtgacagaccacggaagctgcgtgcgggcttgcggagctgactcctacgaaatggaggaagatggcgtgcgcaagtgcaagaaatgtgaggggccatgtcggaaagtgtgcaatgggatcggaattggcgagttcaaggattcactgagcatcaacgccaccaatattaagcacttcaagaactgcacatccatctctggagatctgcacattctgcccgtggccttcagaggcgactcttttactcacaccccacccctggaccctcaggaactggatatcctgaagactgtgaaagagatcaccggcttcctgctgattcaggcctggcctgagaacaggacagacctgcatgcttttgagaatctggaaatcattagagggaggactaagcagcacggacagttcagcctggccgtggtgtccctgaacatcacttctctgggcctgcgcagtctgaaggagatttcagacggcgatgtgatcattagcgggaacaaaaatctgtgctacgccaacaccatcaattggaagaaactgtttgggacatccggacagaagactaaaatcatttctaacaggggagagaatagttgtaaggccacaggccaggtgtgccatgctctgtgctccccagaaggatgctggggaccagagcctcgcgattgcgtgagctgtaggaacgtgtcccgcggcagggaatgcgtggacaagtgcaatctgctggagggggaaccaagagagttcgtggaaaactctgagtgcatccagtgtcaccctgagtgtctgccacaggccatgaacattacatgcactgggaggggacccgacaattgcatccagtgtgcccattacattgatggcccccactgcgtgaagacatgccctgctggagtgatgggcgagaacaatactctggtgtggaaatacgccgacgctggacacgtgtgccacctgtgccaccccaattgtacttatggatgcaccggaccaggactggagggatgccctactaacgggcctaagatcccaagcattgccaccgggatggtgggagctctgctgctgctgctggtggtggccctgggaatcggcctgtttatgcggagaaggcacattgtgagaaaaaggaccctgcgccggctgctgcaggagagggaactggtggaacccctgacaccttctggagaggcccctaaccaggctctgctgcggatcctgaaggagactgagttcaagaagatcaaggtgctggggagtggagcctttggaaccgtgtacaagggcctgtggatcccagagggggaaaaggtgaaaatccccgtggccatcaagacatctcctaaggctaacaaggaaatcctggacgaggcctatgtgatggctagtgtggataacccacacgtgtgccgcctgctgggcatctgcctgacctcaacagtgcagctgattacccagctgatgcccttcgggtgtctgctggactacgtgcgggaacacaaggataacatcggaagccagtatctgctgaattggtgcgtgcagattgccaaaggcatgaattacctggaggacagaaggctggtgcatagagatctggccgctaggaacgtgctggtgaagacaccccagcacgtgaaaatcactgattttggactggccaagctgctgggcgctgaggaaaaagaatatcatgccgagggcgggaaggtgcccatcaagtggatggctctggagagcatcctgcatcggatctacacacaccagagcgacgtgtggtcctatggcgtgaccgtgtgggaactgatgaccttcgggtctaagccctacgacggaatccctgccagtgagatctctagtattctggaaaaaggggagagactgccacagcctccaatctgtaccattgacgtgtatatgatcatggtgaagtgctggatgattgacgccgatagtagaccaaagttcagggaactgatcattgagttttcaaaaatggctcgcgacccccagcggtacctggtcatccagggcgatgaaagaatgcacctgccatctcccaccgacagtaacttctacagggccctgatggacgaggaagatatggacgatgtggtggacgctgatgagtatctgatcccccagcagggcttcttttcaagcccaagtacttcacggacccccctgctgtcctctctgtctgccaccagtaacaattcaacagtggcttgtatcgatcgcaacggcctgcagtcctgcccaattaaggaggactcttttctgcagcggtatagttcagaccccacaggggccctgactgaagattccatcgacgataccttcctgcctgtgccagagtacattaatcagagtgtgccaaagaggccagctggatcagtgcagaaccccgtgtaccacaaccagccactgaatccagctccttcccgggacccacattaccaggatccccactctacagctgtggggaaccctgagtatctgaataccgtgcagccaacatgcgtgaacagcaccttcgacagccctgcccattgggctcagaagggatcccaccagatctctctggacaacccagattaccagcaggacttctttcctaaggaagccaaaccaaacggaatcttcaagggcagcaccgccgaaaatgctgagtatctgagagtggcccctcagagctccgagttcatcggcgctggcggcggcggcagcggcggcggcggctccggcggcggcggctctggcggcaaacctattcctaaccctctgctgggactggactcaactatggcaagtagcgagaatgtgattacagagtttatgaggttcaaagtcaggatggaggggaccgtcaatgggcacgagttcgagattgagggagagggagagggcaggccttatgagggccacaacaccgtgaagctgaaggtgacaaagggaggaccactgcctttcgcctgggacatcctgtctccacagtttcagtatggcagcaaggtgtacgtgaagcacccagccgacatccccgattacaagaagctgtctttccccgagggctttaagtgggagagagtgatgaacttcgaggacggaggagtggcaaccgtgacacaggacagctccctgcaggatggctgctttatctataaggtgaagttcatcggcgtgaattttccttccgatggcccagtgatgcagaagaagaccatgggctgggaggcctctacagagcggctgtaccccagagacggcgtgctgaagggcgagacacacaaggccctgaagctgaaggatggcggccactatctggtggagttcaagagcatctacatggccaagaagcccgtgcagctgcctggctactattacgtggacgccaagctggatatcacatcccacaatgaagattacaccatcgtggagcagtatgaacgcaccgaagggaggcatcacctgtttctgatccggatgatattgtatacgaatccggatgatattgttatcgaa

SEQ ID NO: 54 - RNA, 4425 nt, synthetic construct
  misc_feature 1..4425: note = artificial sequence for RNAi
  source 1..4425: mol_type = other RNA
atgcgaccaagcggaaccgctggagccgctctgctggctctgctggccgccctgtgccctgccagcagggctctggaggaaaagaaagtgtgccaggggacatccaacaagctgacacagctgggaactttcgaggatcactttctgagcctgcagagaatgtttaacaattgcgaagtggtgctgggcaacctggagatcacttacgtgcagaggaattatgacctgagcttcctgaagaccattcaggaagtggccggatacgtgctgatcgctctgaacacagtggaacggatccccctggagaatctgcagatcattaggggcaacatgtactatgagaacagctacgccctggctgtgctgagcaactatgacgccaataagaccgggctgaaagaactgccaatgagaaatctgcaggagatcctgcatggagccgtgaggttctcaaacaatcccgccctgtgcaacgtggagagcatccagtggagggacattgtgagctccgatttcctgagcaacatgtccatggattttcagaatcacctggggtcctgccagaagtgtgacccaagttgtcccaacggatcatgctggggagccggcgaggaaaattgccagaagctgactaaaatcatttgcgctcagcagtgttccggccgctgcagggggaaatctcctagtgattgctgtcataatcagtgtgctgctggatgcaccggaccacgcgaatctgactgcctggtgtgccgcaagtttcgggacgaggccacctgtaaagatacatgcccccctctgatgctgtacaaccccaccacatatcagatggatgtgaatcctgaggggaagtactccttcggagccacctgcgtgaagaagtgccctcgcaactatgtggtgacagaccacggaagctgcgtgcgggcttgcggagctgactcctacgaaatggaggaagatggcgtgcgcaagtgcaagaaatgtgaggggccatgtcggaaagtgtgcaatgggatcggaattggcgagttcaaggattcactgagcatcaacgccaccaatattaagcacttcaagaactgcacatccatctctggagatctgcacattctgcccgtggccttcagaggcgactcttttactcacaccccacccctggaccctcaggaactggatatcctgaagactgtgaaagagatcaccggcttcctgctgattcaggcctggcctgagaacaggacagacctgcatgcttttgagaatctggaaatcattagagggaggactaagcagcacggacagttcagcctggccgtggtgtccctgaacatcacttctctgggcctgcgcagtctgaaggagatttcagacggcgatgtgatcattagcgggaacaaaaatctgtgctacgccaacaccatcaattggaagaaactgtttgggacatccggacagaagactaaaatcatttctaacaggggagagaatagttgtaaggccacaggccaggtgtgccatgctctgtgctccccagaaggatgctggggaccagagcctcgcgattgcgtgagctgtaggaacgtgtcccgcggcagggaatgcgtggacaagtgcaatctgctggagggggaaccaagagagttcgtggaaaactctgagtgcatccagtgtcaccctgagtgtctgccacaggccatgaacattacatgcactgggaggggacccgacaattgcatccagtgtgcccattacattgatggcccccactgcgtgaagacatgccctgctggagtgatgggcgagaacaatactctggtgtggaaatacgccgacgctggacacgtgtgccacctgtgccaccccaattgtacttatggatgcaccggaccaggactggagggatgccctactaacgggcctaagatcccaagcattgccaccgggatggtgggagctctgctgctgctgctggtggtggccctgggaatcggcctgtttatgcggagaaggcacattgtgagaaaaaggaccctgcgccggctgctgcaggagagggaactggtggaacccctgacaccttctggagaggcccctaaccaggctctgctgcggatcctgaaggagactgagttcaagaagatcaaggtgctggggagtggagcctttggaaccgtgtacaagggcctgtggatcccagagggggaaaaggtgaaaatccccgtggccatcaagacatctcctaaggctaacaaggaaatcctggacgaggcctatgtgatggctagtgtggataacccacacgtgtgccgcctgctgggcatctgcctgacctcaacagtgcagctgattacccagctgatgcccttcgggtgtctgctggactacgtgcgggaacacaaggataacatcggaagccagtatctgctgaattggtgcgtgcagattgccaaaggcatgaattacctggaggacagaaggctggtgcatagagatctggccgctaggaacgtgctggtgaagacaccccagcacgtgaaaatcactgattttggactggccaagctgctgggcgctgaggaaaaagaatatcatgccgagggcgggaaggtgcccatcaagtggatggctctggagagcatcctgcatcggatctacacacaccagagcgacgtgtggtcctatggcgtgaccgtgtgggaactgatgaccttcgggtctaagccctacgacggaatccctgccagtgagatctctagtattctggaaaaaggggagagactgccacagcctccaatctgtaccattgacgtgtatatgatcatggtgaagtgctggatgattgacgccgatagtagaccaaagttcagggaactgatcattgagttttcaaaaatggctcgcgacccccagcggtacctggtcatccagggcgatgaaagaatgcacctgccatctcccaccgacagtaacttctacagggccctgatggacgaggaagatatggacgatgtggtggacgctgatgagtatctgatcccccagcagggcttcttttcaagcccaagtacttcacggacccccctgctgtcctctctgtctgccaccagtaacaattcaacagtggcttgtatcgatcgcaacggcctgcagtcctgcccaattaaggaggactcttttctgcagcggtatagttcagaccccacaggggccctgactgaagattccatcgacgataccttcctgcctgtgccagagtacattaatcagagtgtgccaaagaggccagctggatcagtgcagaaccccgtgtaccacaaccagccactgaatccagctccttcccgggacccacattaccaggatccccactctacagctgtggggaaccctgagtatctgaataccgtgcagccaacatgcgtgaacagcaccttcgacagccctgcccattgggctcagaagggatcccaccagatctctctggacaacccagattaccagcaggacttctttcctaaggaagccaaaccaaacggaatcttcaagggcagcaccgccgaaaatgctgagtatctgagagtggcccctcagagctccgagttcatcggcgctggcggcggcggcagcggcggcggcggctccggcggcggcggctctggcggcaaacctattcctaaccctctgctgggactggactcaactatggcaagtagcgagaatgtgattacagagtttatgaggttcaaagtcaggatggaggggaccgtcaatgggcacgagttcgagattgagggagagggagagggcaggccttatgagggccacaacaccgtgaagctgaaggtgacaaagggaggaccactgcctttcgcctgggacatcctgtctccacagtttcagtatggcagcaaggtgtacgtgaagcacccagccgacatccccgattacaagaagctgtctttccccgagggctttaagtgggagagagtgatgaacttcgaggacggaggagtggcaaccgtgacacaggacagctccctgcaggatggctgctttatctataaggtgaagttcatcggcgtgaattttccttccgatggcccagtgatgcagaagaagaccatgggctgggaggcctctacagagcggctgtaccccagagacggcgtgctgaagggcgagacacacaaggccctgaagctgaaggatggcggccactatctggtggagttcaagagcatctacatggccaagaagcccgtgcagctgcctggctactattacgtggacgccaagctggatatcacatcccacaatgaagattacaccatcgtggagcagtatgaacgcaccgaagggaggcatcacctgtttctgatccggatgatattgtatacgaatccggatgatattgttatcgaa

SEQ ID NO: 55 - DNA, 1365 bp, synthetic construct
  misc_feature 1..1365: note = artificial sequence for RNAi
  source 1..1365: mol_type = other DNA
atggcaagctcagaaaatgtcatcaccgagttcatgcgattcaaagtgcgcatggagggcaccgtgaacggccacgagttcgagatcgagggagagggagagggccggccatatgagggccacaataccgtgaagctgaaggtgacaaagggaggaccactgcctttcgcctgggacatcctgtctcctcagtttcagtatggcagcaaggtgtacgtgaagcacccagccgacatccccgattacaagaagctgtccttcccagagggctttaagtgggagagagtgatgaacttcgaggacggaggagtggcaaccgtgacacaggacagctccctgcaggatggctgctttatctataaggtgaagttcatcggcgtgaattttcctagcgatggcccagtgatgcagaagaagaccatgggctgggaggcctccacagagagactgtaccctagggacggcgtgctgaagggcgagacacacaaggccctgaagctgaaggatggcggccactatctggtggagtttaagagcatctacatggccaagaagcccgtgcagctgcctggctactattacgtggacgccaagctggatatcacctcccacaacgaggattatacaatcgtggagcagtacgagcgcaccgagggccggcaccacctgttcctgggcggcggcggctctggaggaggaggaagcggaggaggaggatccggcatgacagagtacaagctggtggtggtgggagccagaggcgtgggcaagtccgccctgaccatccagctgatccagaatcactttgtggacgagtatgatcccacaatcgaggactcttacaggaagcaggtggtcatcgatggcgagacatgcctgctggacatcctggatacagccggccaggaggagtattctgccatgcgggaccagtacatgaggaccggcgagggcttcctgtgcgtgttcgccatcaacaatacaaagagcttcgaggatatccaccactatcgcgagcagatcaagcgggtgaaggactctgaggatgtgccaatggtgctggtgggcaacaagtgtgacctgcccagcagaaccgtggacacaaagcaggcccaggatctggccaggtcctacggcatccctttcatcgagacaagcgccaagacacggcagagagtggaggatgccttttataccctggtgagggagatccgccagtaccggctgaagaaaatctcaaaggaagaaaagacccctggctgcgtgaaaattaagaaatgtattattatgtgatccggatgatattgtatacgaatccggatgatgttttaatcgaatccggatgatattgttatcgaactcgag

SEQ ID NO: 56 - RNA, 1365 nt, synthetic construct
  misc_feature 1..1365: note = artificial sequence for RNAi
  source 1..1365: mol_type = other RNA
atggcaagctcagaaaatgtcatcaccgagttcatgcgattcaaagtgcgcatggagggcaccgtgaacggccacgagttcgagatcgagggagagggagagggccggccatatgagggccacaataccgtgaagctgaaggtgacaaagggaggaccactgcctttcgcctgggacatcctgtctcctcagtttcagtatggcagcaaggtgtacgtgaagcacccagccgacatccccgattacaagaagctgtccttcccagagggctttaagtgggagagagtgatgaacttcgaggacggaggagtggcaaccgtgacacaggacagctccctgcaggatggctgctttatctataaggtgaagttcatcggcgtgaattttcctagcgatggcccagtgatgcagaagaagaccatgggctgggaggcctccacagagagactgtaccctagggacggcgtgctgaagggcgagacacacaaggccctgaagctgaaggatggcggccactatctggtggagtttaagagcatctacatggccaagaagcccgtgcagctgcctggctactattacgtggacgccaagctggatatcacctcccacaacgaggattatacaatcgtggagcagtacgagcgcaccgagggccggcaccacctgttcctgggcggcggcggctctggaggaggaggaagcggaggaggaggatccggcatgacagagtacaagctggtggtggtgggagccagaggcgtgggcaagtccgccctgaccatccagctgatccagaatcactttgtggacgagtatgatcccacaatcgaggactcttacaggaagcaggtggtcatcgatggcgagacatgcctgctggacatcctggatacagccggccaggaggagtattctgccatgcgggaccagtacatgaggaccggcgagggcttcctgtgcgtgttcgccatcaacaatacaaagagcttcgaggatatccaccactatcgcgagcagatcaagcgggtgaaggactctgaggatgtgccaatggtgctggtgggcaacaagtgtgacctgcccagcagaaccgtggacacaaagcaggcccaggatctggccaggtcctacggcatccctttcatcgagacaagcgccaagacacggcagagagtggaggatgccttttataccctggtgagggagatccgccagtaccggctgaagaaaatctcaaaggaagaaaagacccctggctgcgtgaaaattaagaaatgtattattatgtgatccggatgatattgtatacgaatccggatgatgttttaatcgaatccggatgatattgttatcgaactcgag

SEQ ID NO: 57 - DNA, 759 bp, synthetic construct
  misc_feature 1..759: note = artificial sequence for RNAi
  source 1..759: mol_type = other DNA
ggaggtggatcgggaggtggatcgcctaaagatccagccaaacctccggccaaggcacaagttgtgggatggccaccggtgagatcataccggaagaacgtgatggtttcctgccaaaaatcaagcggtggcccggaggcggcggcgttcgtgaagagcgctgggaagcccatccccaaccccctgctgggcctggacagcacgggcagcggcgccaccaacttcagcctgctgaagcaggccggcgacgtggaggagaaccccggccccatgaagaccttcaacatctctcagcaggaccttgagctggtggaagttgccactgagaaaatcaccatgctctatgaggacaacaagcaccatgttggtgctgccatcaggaccaagacaggagaaatcatctctgctgtccacattgaagcctacattggcagggtcactgtgtgtgcagaggccattgccattgggtctgcagtctccaatgggcagaaggactttgacaccattgtggctgtgaggcacccctacagtgatgaggtggacaggtccatcagagtggtgtccccctgtggcatgtgcagggagctcatctcagactatgcccctgattgctttgttctgattgagatgaatggcaagctggtcaagacaaccattgaggagctgattccactgaaatacaccagaaactgatccggatgatattgtatacgaatccggatgatgttttaatcgaatccggatgatattgttatcgaa

SEQ ID NO: 58 - RNA, 759 nt, synthetic construct
  misc_feature 1..759: note = artificial sequence for RNAi
  source 1..759: mol_type = other RNA
ggaggtggatcgggaggtggatcgcctaaagatccagccaaacctccggccaaggcacaagttgtgggatggccaccggtgagatcataccggaagaacgtgatggtttcctgccaaaaatcaagcggtggcccggaggcggcggcgttcgtgaagagcgctgggaagcccatccccaaccccctgctgggcctggacagcacgggcagcggcgccaccaacttcagcctgctgaagcaggccggcgacgtggaggagaaccccggccccatgaagaccttcaacatctctcagcaggaccttgagctggtggaagttgccactgagaaaatcaccatgctctatgaggacaacaagcaccatgttggtgctgccatcaggaccaagacaggagaaatcatctctgctgtccacattgaagcctacattggcagggtcactgtgtgtgcagaggccattgccattgggtctgcagtctccaatgggcagaaggactttgacaccattgtggctgtgaggcacccctacagtgatgaggtggacaggtccatcagagtggtgtccccctgtggcatgtgcagggagctcatctcagactatgcccctgattgctttgttctgattgagatgaatggcaagctggtcaagacaaccattgaggagctgattccactgaaatacaccagaaactgatccggatgatattgtatacgaatccggatgatgttttaatcgaatccggatgatattgttatcgaa

SEQ ID NO: 59 - DNA, 97 bp, synthetic construct
  misc_feature 1..97: note = artificial sequence for RNAi
  source 1..97: mol_type = other DNA
tgctgttgacagtgagcgcacgacgagaacagttgaaacatagtgaagccacagatgtatgtttcaactgttctcgtcgtttgcctactgcctcgga

SEQ ID NO: 60 - RNA, 97 nt, synthetic construct
  misc_feature 1..97: note = artificial sequence for RNAi
  source 1..97: mol_type = other RNA
tgctgttgacagtgagcgcacgacgagaacagttgaaacatagtgaagccacagatgtatgtttcaactgttctcgtcgtttgcctactgcctcgga

SEQ ID NO: 61 - DNA, 97 bp, synthetic construct
  misc_feature 1..97: note = artificial sequence for RNAi
  source 1..97: mol_type = other DNA
tgctgttgacagtgagcgactcgtgaaatcccgttagtaatagtgaagccacagatgtattactaacgggatttcacgaggtgcctactgcctcgga

SEQ ID NO: 62 - RNA, 97 nt, synthetic construct
  misc_feature 1..97: note = artificial sequence for RNAi
  source 1..97: mol_type = other RNA
tgctgttgacagtgagcgactcgtgaaatcccgttagtaatagtgaagccacagatgtattactaacgggatttcacgaggtgcctactgcctcgga

SEQ ID NO: 63 - DNA, 97 bp, synthetic construct
  misc_feature 1..97: note = artificial sequence for RNAi
  source 1..97: mol_type = other DNA
tgctgttgacagtgagcgcaggaattataatgcttatctatagtgaagccacagatgtatagataagcattataattcctatgcctactgcctcgga

SEQ ID NO: 64 - RNA, 97 nt, synthetic construct
  misc_feature 1..97: note = artificial sequence for RNAi
  source 1..97: mol_type = other RNA
tgctgttgacagtgagcgcaggaattataatgcttatctatagtgaagccacagatgtatagataagcattataattcctatgcctactgcctcgga

SEQ ID NO: 65 - DNA, 97 bp, synthetic construct
  misc_feature 1..97: note = artificial sequence for RNAi
  source 1..97: mol_type = other DNA
tgctgttgacagtgagcgaccagctaaaggtgaagatatatagtgaagccacagatgtatatatcttcacctttagctggctgcctactgcctcgga

SEQ ID NO: 66 - RNA, 97 nt, synthetic construct
  misc_feature 1..97: note = artificial sequence for RNAi
  source 1..97: mol_type = other RNA
tgctgttgacagtgagcgaccagctaaaggtgaagatatatagtgaagccacagatgtatatatcttcacctttagctggctgcctactgcctcgga

SEQ ID NO: 67 - DNA, 97 bp, synthetic construct
  misc_feature 1..97: note = artificial sequence for RNAi
  source 1..97: mol_type = other DNA
tgctgttgacagtgagcgacagctaaaggtgaagatatattagtgaagccacagatgtaatatatcttcacctttagctggtgcctactgcctcgga

SEQ ID NO: 68 - RNA, 97 nt, synthetic construct
  misc_feature 1..97: note = artificial sequence for RNAi
  source 1..97: mol_type = other RNA
tgctgttgacagtgagcgacagctaaaggtgaagatatattagtgaagccacagatgtaatatatcttcacctttagctggtgcctactgcctcgga

(19) United States
(12) Patent Application Publication
(10) Pub. No.: US 2025/0051759 A1
(43) Pub. Date: Feb. 13, 2025

(54) METHODS AND MOLECULES FOR RNA INTERFERENCE (RNAi)

(71) Applicant: BOEHRINGER INGELHEIM INTERNATIONAL GMBH, Ingelheim am Rhein (DE)

(72) Inventors: Ralph NEUMÜLLER, Ingelheim am Rhein (DE); Johannes ZUBER, Ingelheim am Rhein (DE); Thomas HOFFMANN, Ingelheim am Rhein (DE); Matthias HINTERNDORFER, Ingelheim am Rhein (DE)

(73) Assignee: BOEHRINGER INGELHEIM INTERNATIONAL GMBH, Ingelheim am Rhein (DE)

(21) Appl. No.: 18/721,618

(22) PCT Filed: Dec. 23, 2022
(86) PCT No.: PCT/EP2022/087671

§ 371 Date: Jun. 18, 2024

(30) Foreign Application Priority Data
Dec. 23, 2021 (EP) 21217407.2

(51) Int. Cl. C12N 15/10; C12N 15/113

(52) U.S. Cl. CPC C12N 15/1082; C12N 15/1135; C12N 2310/141; C12N 2310/531

(57) ABSTRACT

The invention provides a method of suppressing gene expression, comprising providing a mammalian cell with a suppression target RNA molecule comprising a heterologous RNAi target site operatively linked to a RNA sequence from a gene of interest, or with a DNA encoding said suppression target RNA molecule; providing to the cell an inhibitory RNA that has a complementary nucleotide sequence to the heterologous RNAi target site; wherein binding of the inhibitory RNA to the heterologous RNAi target site suppresses gene expression of the operatively linked RNA sequence from the gene of interest; and wherein said heterologous RNAi target site has a size of at least 18 nucleotides in length. The invention further provides nucleic acids and cells suitable for such a method.

### TECHNICAL FIELD

The present invention relates to methods of suppressing the expression of target genes in mammalian cells.

### BACKGROUND ART

Drug development is guided by genetic loss-of-function (LOF) experiments that validate a therapeutic genetic target and benchmark expected activities of a candidate drug. The toolbox of genetic loss-of-function methods has grown over the past decades (1,2).

RNA interference (RNAi) has become a widespread technology with multiple applications (3-5). RNA interference is e.g. described in EP 2 267 131 B1 includes genetic inhibition with double-stranded RNA without length limitation.

EP 1 309 726 B2, EP 1407044 B2 and others describe isolated small interfering RNA (siRNA) of approximately 21-23 nucleotides in length, and how these siRNAs mediate RNAi of a corresponding mRNAs and inactivate corresponding genes by transcriptional silencing.

EP 1 546 174 B1 describes that short hairpin RNAs (shRNAs) can be engineered to suppress the expression of desired genes. shRNAs can be synthesized exogenously or can be transcribed in cells, thus permitting the construction of continuous cell lines or transgenic animals in which RNAi enforces stable gene silencing.

WO 2014/117050 A2 describes microRNA-adapted shRNAs (shRNAmir) that are superior to siRNA and conventional shRNA and the optimization of the miR-E shRNAmir backbone. shRNA sequences can be cloned and used in this or a related (mir30) backbone (12).

Mohr et al. give an overview of gene silencing through sequence-specific targeting of mRNAs by RNAi and its application in genome-wide functional screens in cultured cells and in vivo in model organisms. These screens have resulted in the identification of new cellular pathways and potential drug targets (1).

In Drosophila, an alternative strategy of RNAi-based loss-of-function studies, specifically the generation of shRNAs targeting green fluorescent protein (GFP), was described. GFP was randomly inserted into a small set of genes using a synthetic exon strategy into fly genes by an international research collaboration (Kelso et al., Nucleic Acids Research 2004). The GFP specific shRNAs were subsequently used in transgenic fly strains to knock-down GFP-tagged genes. This work demonstrated that non-optimized shRNAs targeting an exogenous sequence (GFP) can be used for knock-down studies in the fly but not in mammals (13).

Further, the target sequence of the shRNA targeting GFP was utilized and inserted into target genes using CRSIPR/Cas9. Subsequently, these target genes (Tif-1a, Lola, MESR4) could be knocked down in transgenic flies. The sequences used in this study were not optimized for usage in human or mouse cells (14). In general, the concept of utilizing an exogenous sequence as an off-target free RNAi effector sequence for potent knock-down has not been established in mammalian cells.

WO 2006/137941 A2 describes an investigation of artificial miRNAs that target endogenous sequences of a cell.

De Guire et al., Nucleic Acids Research 38(13) (2010): 1-8, describes artificial miRNAs with genetic target sites that are endogenous to the investigated cells. In an in si/ico investigation mutated target sites were used to compute average hybridization affinities.

Atsushi Shibata et al., Journal of RNAi and gene silencing 3 (1)(2007): 237-247, describes an artificial miRNA, which incorporates restriction enzyme recognition sequences in the loop and stem region of the miRNA precursor structure. These precursors were processed into mature miRNAs. Dicistronic miRNA precursor clusters targeting multiple sites within a single mRNA could suppress highly-mutable targets, such as in HIV.

Wang et al., ACS Synthetic Biology 11 (2015): 1193-1200), describes synthetic miRNA for multiplex RNA interference in mammalian cells in order to study miRNA maturation. A reporter system with a reporter protein fused to target sites was used.

Huesken et al., Nature Biotechnology 23(8) (2005): 995-1001 describes the design of siRNAs using an artificial neural network. A collection of 48,746 siRNA targeting 24,373 genes was generated and used to identify genes involved in the cellular response to hypoxia. Only a few hits are described for this screen.

Other important technologies include CRISPR (Clustered Regularly Interspaced Short Palindromic Repeats), and site-specific recombination technologies such as the Cre-Lox or Flp-FRT systems (1,2).

### SUMMARY OF INVENTION

### Technical Problem

While the prior art technologies have revolutionized genetic screening, target identification and validation, each method is associated with drawbacks that limit the usability in certain aspects of target validation.

There is a need to improve RNAi based methods, in particular to reduce off-target effects and increase knock-down levels.

Optimally, RNAi based methods would be inducible and fully reversible methods that are well suited to guide drug development projects and that have certain advantages compared to the methods known in the art. In particular, the loss-of-function should be induced homogenously in all treated cells and the method should not be associated with off-target effects. In addition, the method should be applicable to all target classes of genes, ranging from protein coding genes to regulatory RNAs and should allow for the evaluation of untagged or unaltered coding sequences.

This need is addressed by the embodiments provided herein. According to the present invention there is provided a method of suppressing the expression of target genes in mammalian cells as set forth in claim 1.

### Solution to Problem

It has been found that the technical problem outlined above is solved by the methods according to the invention (see claim 1):

The invention provides a method of suppressing gene expression, comprising providing a mammalian cell with a suppression target RNA molecule comprising a heterologous RNAi target site operatively linked to a RNA sequence from a gene of interest, or with a DNA encoding said suppression target RNA molecule; providing to the cell an inhibitory RNA that has a complementary nucleotide sequence to the heterologous RNAi target site; wherein binding of the inhibitory RNA to the heterologous RNAi target site suppresses gene expression of the operatively linked RNA sequence from the gene of interest; and wherein said heterologous RNAi target site has a size of at least 18 nucleotides in length.

The invention further provides a nucleic acid comprising the nucleic acid sequence 5′-TTCGWWWNNAHHWWCATCCGGN-3′ (SEQ ID NO: 1), wherein W is A or T, H is A or T or C, and N is A or T or G or C; wherein A is adenine, C is cytosine, G is guanine, T is thymine in DNA or uracil in RNA.

The invention further provides a nucleic acid comprising the nucleic acid sequence 5′-NCCGGATGWWDDTNNWWWCGAA-3′ (SEQ ID NO: 27), wherein W is T or A, D is T or A or G, and N is T or A or C or G.

Also provided is a nucleic acid comprising a promoter, an expression sequence and a nucleic acid sequence of a nucleic acid according to the invention.

Further provided is a vector comprising a nucleic acid of the invention.

Also provided is a mammalian cell comprising a DNA encoding a gene of interest operatively linked to a sequence of a heterologous RNAi target site; further comprising a DNA encoding an inhibitory RNA that has a complementary region to the heterologous RNAi target site; wherein said heterologous RNAi target site has a size of at least 18 nucleotides in length. Also provided is a mammalian cell comprising a nucleic acid or vector of the invention.

Further provided is a method of determining the effects of a loss-of-function of a gene of interest in a mammalian cell, comprising providing a mammalian cell with a DNA that transcribes a suppression target RNA molecule comprising a heterologous RNAi target site operatively linked to a RNA sequence from the gene of interest; providing to the cell an inhibitory RNA that has a complementary nucleotide sequence to the heterologous RNAi target site; wherein binding of the inhibitory RNA to the heterologous RNAi target site suppresses gene expression of the operatively linked RNA sequence from the gene of interest; and wherein said heterologous RNAi target site has a size of at least 18 nucleotides in length; and observing a change of phenotype of the mammalian cell between the mammalian cells with the provided inhibitory RNA and without providing the inhibitory RNA.

All aspects of the invention are related and can be combined unless explicitly stated otherwise. For example, the nucleic acids, vectors and cells can be used in the inventive methods. The inventive nucleic acids, vectors and cells can be required to be suitable for being used in the inventive methods. All embodiments, preferred options and detailed descriptions of aspects and embodiments of the invention can be combined with each other and with the methods, nucleic acids, vectors and cells.

### BRIEF DESCRIPTION OF DRAWINGS

FIG. 1: WebLogo (Berkeley) was used to generate the respective sequence logo displaying nucleotide position biases of 2161 shRNAs with exceptionally high DSIR (Designer of Small Interfering RNA; Vert et al., BMC Bioinformatics 2006) scores (>105).

FIG. 2: Nucleotide biases in top-scoring DSIR predictions and derived design matrix. Selection criteria were based on nucleotide biases in top-scoring DSIR predictions. Black bar, seed region used for off-target predictions.

FIG. 3: WebLogo (Berkeley) was used to generate the respective sequence logo displaying nucleotide position biases of seed sequences (guide position 2-8) with the lowest off-target score in siSPOTR analysis (top 1%).

FIG. 4: WebLogo (Berkeley) was used to generate the respective sequence logo displaying position biases in sequences from (FIG. 3) harboring a T in guide position 2.

FIG. 5: Graphical representation of used vectors. The GPT target sequence contains either of the selected ARTi target sequences.

FIG. 6: Knockdown efficiency of ARTi-shRNAmirs. Flow cytometric quantification of GFP knockdown efficiency in immortalized MEFs 2 days after transduction with ARTi-shRNAmir or control shRNAs. Percent knowdown is normalized to shRen713 control (targeting Renilla luciferase).

FIG. 7: Toxicity of ARTi-shRNAmirs profiled in competitive cell proliferation assays. Competitive co-culture assays of three cell lines after transduction with ARTi-shRNAmirs, shRen713 (targeting Renilla luciferase) or shMyc1834 (c-Myc) control. RN2 cells showed a considerable growth disadvantage in the presence of ARTi.6634.

FIG. 8: Transcriptional profiling. Principal component analysis of the 100 most differentially expressed genes in three cell lines expressing ARTishRNA-mirs or empty vector control.

FIG. 9: Number of downregulated genes (FC≥2, TPM≥10) in ARTi-shRNAmir expressing cells compared to empty vector control.

FIG. 10: Schematic drawing of EGFR^{del19}-ARTi construct ectopically expressed in PC-9 cells. EGFR^{del19}-ARTi dependent PC-9 cells are generated by knockout of endogenous EGFR in the background of EGFR^{del19}-ARTi expressing PC-9 cells.

FIG. 11: Functional validation of EGFR^{del19} construct in Ba/F3 cells dependent on interleukin-3 (IL3) (parental), EGFR^{del19} or EGFR^{del19,C797s} oncogenic variants treated with indicated compounds. Values represent the IC₅₀ in nM. As EGFR^{del19}-ARTi expressing Ba/F3 cells survive in the absence of IL3, the EGFR^{del19}-ARTi construct is functional.

FIG. 12: Western blot analysis confirming overexpression of EGFR^{del19}-ARTi construct (as described in FIG. 9), knockout of endogenous EGFR and doxycycline induced knockdown or EGFR^{del19}-ARTi.

FIG. 13: Proliferation assay and subsequent crystal violet staining of parental and engineered PC-9 cells in absence or presence of doxycycline.

FIG. 14: Expression levels (y-axis: normalized counts) of the EGFR^{del19}-ARTi construct in parental and engineered PC-9 cells after 4 and 8 days of doxycycline treatment or no treatment.

FIG. 15: Expression levels (y-axis: normalized counts) of DUSP6 in parental and engineered PC-9 cells after 4 and 8 days of doxycycline treatment or no treatment.

FIG. 16: In vivo experiment comparing doxycycline induced ARTi-shRNAmir transduced PC-9 cells to drinking water control.

FIG. 17: In vivo experiment comparing doxycycline induced EGFR^{del19}-ARTi knockdown to Osimertinib-mediated inhibition of^{EGFRdel19} oncogenic signaling.

FIG. 18: Schematic drawing of MIA PaCa-2 genome engineering strategy.

FIG. 19: Western blot for KRAS (dsRed::KRAS^{G12R}, upper band), Actin (magenta) and KRAS^{G12C} (lower band) (upper plot) and dsRed (upper band) and Actin (lower band) (bottom plot) for different engineered MIA PaCa-2 cells in the presence and absence of doxycycline.

FIG. 20: Proliferation assay and subsequent crystal violet staining of parental and engineered MIA PaCa-2 cells in absence or presence of doxycycline.

FIG. 21: Growth curves of tumors implanted with engineered MIA PaCa-2 cells (overexpression of ARTi-shRNAmir and dsRed::KRAS^{G12R}-ARTi; KRAS^{G12C} proficient) in the absence and presence of doxycycline.

FIG. 22: Growth curves of tumors implanted with engineered MIA PaCa-2 cells (overexpression of ARTi-shRNAmir and dsRed::KRAS^{G12R}-ARTi; KRAS^{G12C} deficient (knockout)) in the absence and presence of doxycycline.

FIG. 23: Schematic drawing of C-terminal tagging of STAG1 at endogenous locus to generate a homozygous STAG1-ARTi allele. (AID=degron sequence for auxin induced degradation, V5=V5 tag derived fom the P and V protein of the simian virus 5, P2A=2A self-cleaving peptide from porcine teschovirus-1, Blasti=blasticidine resistance gene).

FIG. 24: Schematic drawing of lethal paralog interaction between STAG1 and STAG2. Cells survive loss of either paralog but are incapable of growing upon combined loss of STAG1 and STAG2.

FIG. 25: Proliferation assay and subsequent crystal violet staining of parental and engineered HCT 116 cells in absence or presence of doxycycline. Cells lacking SATG2 and treated with doxycycline for ARTi-mediated STAG1 knockdown show impaired proliferation when compared to STAG2 proficient cells or the non-doxycycline treated control.

FIG. 26: Western blot confirmation of STAG1 CRISPR/Cas9-mediated knock-in and knockout of STAG2 in the presence and absence of doxycycline.

FIG. 27: Quantification of tumor growth inhibition (TGI) of an in vivo experiment using STAG2 knock out cells transduced with the ARTi-shRNAmir and homozygeous insertion of the ARTi target sequence at the endogenous STAG1 locus in HCT 116 cells. Addition of doxycycline to the drinking water results in tumor stasis.

FIG. 28: Growth curves of tumors implanted with STAG2 wild type, ARTi-shRNAmir transduced HCT 116 cells that contain a homozygous insertion of the ARTi target sequence at the endogenous STAG1 locus. Similar growth kinetics are observed upon absence or presence of doxycycline in the drinking water.

### DESCRIPTION OF EMBODIMENTS

The invention as a main aspect provides a method of suppressing gene expression. The method comprises the steps of providing a mammalian cell with a suppression target RNA molecule comprising a heterologous RNAi target site operatively linked to a RNA sequence from a gene of interest, or with a DNA encoding said suppression target RNA molecule; and providing to the cell an inhibitory RNA that has a complementary nucleotide sequence to the heterologous RNAi target site. Binding of the inhibitory RNA to the heterologous RNAi target site suppresses gene expression of the operatively linked RNA sequence from the gene of interest. Accordingly, the method uses conditions, wherein in the cell such biding is not prevented and allowed to happen. The heterologous RNAi target site should have a size of at least 18 nucleotides in length, e.g. 18 to 30 nucleotides in length.

One of the main uses of the inventive method is in loss-of-function (LOF) experiments. Expression of a gene of interest can be suppressed or knocked-down, which leads to the loss-of-function of the gene. A change in the mammalian cell due to the loss-of-function can be monitored or observed. Such a change may be a functional alteration, a phenotypic alteration, a morphological alteration, a metabolic alteration, altered cell survival or an effect on gene expression that may be affected by the loss-of-function of the gene of interest that is targeted by the inventive suppression of expression with the inhibitory RNA. Such alterations are in comparison to the same cell or cell type without the inventive suppression of gene expression under the same conditions.

The inventive methods use mammalian cells, which are eukaryotic cells that have a natural RNA inhibition mechanism, such as RNAi as described in the background section. RNAi may require enzymes, such as dicer and proteins of the RNA-induced silencing complex (RISC) and the RISC-Loading Complex (RLC) to be active and/or perform its function in RNAi. Such enzymes and proteins may be native to the mammalian cell or recombinant.

The mammalian cell may be a rodent cell, a primate cell, a mouse cell, a rat cell, a hamster cell, a human cell, a non-human cell, etc. Preferably the mammalian cell is of a cell line from a mammalian species that can be cultured in vitro or that can be implanted into a living organism (in vivo). Included in the present invention is the use of a population or a single cell.

Preferably the cell is an isolated cell and/or not in an animal. The cell may be a cultured cell of a cell culture. Related thereto any of the inventive methods may be an in vitro method. In other embodiments, the cell may be in vivo, which allows in vivo studies of gene function with the inventive method. Included is the use of the mammalian cell, including cells from a cell culture in an organism, e.g. implanted in a mammalian organism.

Any inventive method may exclude any method practiced as a method for treatment of the human or animal body by surgery or therapy. Also excluded may be a diagnostic method practiced on the human or animal body.

The inventive method comprises the step of providing a mammalian cell with a suppression target RNA molecule comprising a heterologous RNAi target site operatively linked to a RNA sequence from a gene of interest. The RNA sequence from a gene of interest is the expressed gene or transcript of a gene that is to be suppressed by the inventive method. This RNA sequence may be a mRNA. It is also possible that the inventive method is used to suppress functional RNAs, such as miRNAs as embodiments of the suppression target RNA molecule. The RNAi target site refers to a sequence in a target gene of interest, to which the (complementary) nucleotide sequence of the inhibitory RNA binds to elicit translational repression or RNA cleavage. The RNAi target site may also be bound by proteins of the RNAi machinery (RNA loaded silencing complex).

The gene of interest, or “target gene”, can be any gene of the mammalian cell or a transgene. Any gene can be turned into a target gene by engineering the target site of the respective gene to include the heterologous RNAi target site, in particular to include the heterologous RNAi target site into its transcribed sequence (e.g. coding or untranslated mRNA regions). It is also possible to use more than one target gene in one reaction. Accordingly, more than one, e.g. 2, 3, 4, 5, 6 or more genes of interest may comprise a heterologous RNAi target site. It may be a different heterologous RNAi target site for the more than one gene of interest, which would allow targeting the genes individually by corresponding inhibitory RNAs, or the heterologous RNAi target site may be the same for the more than one gene of interest so that more than one gene can be suppressed in its expression at the same time. The use of more than one gene of interest with the inventive heterologous RNAi target site permits highly stringent loss-of-function experiments for multiple targets. Isoform-specific integration of the RNAi target site allows for reliable knockdown of specific gene isoforms. Engineering RNAi target site into multiple genes (e.g. paralogs) enables effective combinatorial loss-of-function perturbations without increasing the risk for off-target effects.

This suppression target RNA molecule contains a heterologous RNAi target site. A RNAi target site is a short sequence, e.g. 18 nucleotides (nt) in length or more, to which an inhibitory RNA binds to (see next step of the inventive method). Such a RNAi target site is heterologous according to the invention. Heterologous means that the sequence of the RNAi target site is one that is not naturally found in the mammalian cell. It is a foreign, artificial sequence to the mammalian cell, i.e. not found elsewhere in the genome of the mammalian cell, unless introduced artificially. The heterologous RNAi target site may thus be an exogenous RNAi target site and/or a recombinant RNAi target site. “Exogenous” means that the sequences has been introduced from outside the cell. “Recombinant” refers to a genetic entity distinct from that generally found in nature. As applied to a nucleotide sequence or nucleic acid molecule, this means that said suppression target RNA molecule is the product of various combinations of cloning, restriction and/or ligation steps, and/or other procedures that result in the production of a nucleic acid sequence that is distinct from a sequence or molecule found in the mammalian cell. The heterologous RNAi target site does not match any transcribed genes in the mammalian cell's genome. The use of a heterologous RNAi target site has the benefit of reducing off-target effects in the mammalian cells since the inhibitory RNA has a reduced capability to bind to sequences of the transcriptome of the mammalian cell. The heterologous RNAi target site may be in an endogenous gene or it can be included in overexpression constructs or vectors.

Due to the artificial nature of using the heterologous sequence, the invention is also referred to a “artificial RNAi” (abbreviated “ARTi”) herein.

The present invention achieves a suppression of gene expression through RNA-dependent gene-silencing. This may involve inhibition of translation and/or target RNA cleavage by RNA interference resulting in a reduction in gene expression.

Providing to the cell with a RNA molecule, either the suppression target RNA molecule or the inhibitory RNA or both, may comprise delivering the RNA molecule to the cell or delivering a DNA encoding said RNA molecule to the cell. The DNA may express the RNA molecule to provide it to the cell. Delivering a RNA molecule or DNA to the cell includes but is not limited to transfection, electroporation, microinjection, bacterial-mediated delivery or viral-mediated delivery, viral based transduction, transfection of synthetic RNAs or transfection of plasmids. A DNA may be delivered in form of a vector. E.g. the inventive method may comprise introducing into the cell an expression vector comprising a sequence encoding the suppression target RNA molecule or the inhibitory RNA or both. The suppression target RNA molecule or the inhibitory RNA or both, is then expressed in the cell. An expression vector may comprise one or more transcriptional regulatory sequences, e.g. a promoter, operably linked to the sequence encoding the suppression target RNA molecule or the inhibitory RNA, respectively. Preferred expression vectors for the inhibitory RNA are selected from a retroviral or lentiviral expression vector, a microRNA-based shRNA expression vector, a miR-30 or miR-E expression vector. Another vector may be an integration vector, which integrates a DNA sequence of the suppression target RNA molecule or the inhibitory RNA or both into the genome and/or into a chromosome of the mammalian cell. The DNA encoding the suppression target RNA molecule or the inhibitory RNA may be on separate vectors. In preferred embodiments, the DNA encoding said suppression target RNA molecule is an expression vector or a genomic DNA of the mammalian cell. In particular preferred embodiments, the inhibitory RNA is expressed from a DNA encoding said inhibitory RNA in said mammalian cell. The invention thus also provides a vector comprising a nucleic acid of the invention as DNA.

Preferably expression of the inhibitory RNA is inducible expression by an inducible promoter. E.g. an expression vector may comprise an inducible promoter. A preferred inducible promoter is a tetracycline-responsive element promoter, which may be on the DNA encoding said inhibitory RNA for expression of the inhibitory RNA. Expression by a tetracycline-responsive element promoter may e.g. be controlled by providing the cell with a tetracycline, such as doxycycline.

The heterologous RNAi target site is a RNA interference matching sequence in a target gene of interest. The heterologous RNAi target site should be amenable to RNA interference, such as by comprising a sequence to which the RNAi machinery (RNA loaded silencing complex) binds to elicit translational repression or RNA cleavage. This embodiment is particularly preferred and emphasized to be combined with all other aspects and embodiments of the invention. The target site is characterized by regions of sequence complementarity to the inhibitory RNA.

The heterologous RNAi target site may have a size of at least 18 nt (nucleotides) in length, e.g. 18 nt, 19 nt, 20 nt, 21 nt, 22 nt, 23 nt, 24 nt, 25 nt, 26 nt, 27 nt, 28 nt, 29 nt or 30 nt or more, in length. Preferred is a length of the heterologous RNAi target site of 20 to 25 bases in length, especially 21 to 23 bases in length, even more preferably 22 bases in length.

Alternatively, or in combination with, providing the cells with the suppression target RNA, it is possible to provide the cells with a DNA encoding said suppression target RNA molecule. The DNA may lead to the transcription of the suppression target RNA molecule, which provides said suppression target RNA molecule to the cell. The suppression target RNA molecule can be a transcript, an RNA molecule produced by transcriptional events. The transcript should be amenable to RNA interference.

The inventive method further comprises the step of providing to the cell an inhibitory RNA that has a complementary nucleotide sequence to the heterologous RNAi target site. The complementary nucleotide sequence is also referred to as “guide sequence” herein, in analogy to RNAi terminology. RNAi is a preferred method for suppressing gene expression according to the invention. Similar to the heterologous RNAi target site, the complementary nucleotide sequence can have a size of at least 18 nt (nucleotides) in length, e.g. 18 nt, 19 nt, 20 nt, 21 nt, 22 nt, 23 nt, 24 nt, 25 nt, 26 nt, 27 nt, 28 nt, 29 nt or 30 nt or more, in length. Preferred is a length of the complementary nucleotide sequence to the heterologous RNAi target site of 20 to 25 bases in length, especially 21 to 23 bases in length, even more preferably 22 bases in length.

The inhibitory RNA, preferably a RNAi molecule, comprises a complementary nucleotide sequence to the heterologous RNAi target site, which in itself is heterologous to the mammalian cell. Consequently, the complementary sequence on the inhibitory RNA is also heterologous to the mammalian cell. The inhibitory RNA can be an artificial RNA molecule. The term artificial here refers to a non-naturally occurring RNA interference sequence. In embodiments of the invention the inhibitory RNA comprises a sequence that has been optimized to confer potent knockdown and no detectable off-target effects in mice and human cells. Such an artificial inhibitory RNA is non-naturally occurring in humans or mice as the sequence. Inventive sequences of such embodiments are derived from design choices and subsequent experimental optimization.

Binding of the inhibitory RNA to the heterologous RNAi target site suppresses gene expression of the operatively linked RNA sequence from the gene of interest. Accordingly, the method uses conditions wherein in the cell such binding is not prevented and allowed to happen. This binding of the inhibitory RNA to the heterologous RNAi target site can be sequence specific. This includes sequences with perfect or imperfect sequence complementarity between the heterologous RNAi target site and the complementary sequence of the inhibitory RNA. Complementary is sufficient to elicit suppression of expression in a sequence specific manner of the suppression target RNA molecule(s) with the heterologous RNAi target site that binds the inhibitory RNA. Binding means formation of RNA:RNA nucleotide hybridization or base pairing. Such RNA:RNA hybridization between the heterologous RNAi target site and the inhibitory RNA is preferably by at least 18 bp (base pairs), preferably 18 bp, 19 bp, 20 bp, 21 bp, 22 bp or 23 bp, 24 bp, 25 bp, or more at the heterologous RNAi target site. In particular preferred is a complementarity over 21 to 23 bases in length, especially preferred 22 bases in length.

Preferably, the inhibitory RNA comprises a double strand, wherein one strand of the double strand is complementary to the heterologous RNAi target site. Double stranded nucleic acids may be processed by the RNAi pathway in the mammalian cell, including processing by the enzyme dicer. Such processing leads the way for efficient suppression of the target gene expression. In particular preferred embodiments, the (first) strand that is complementary to the heterologous RNAi target site may be referred to as “guide strand” and the other (or second) strand of the double strand of the inhibitory RNA to this guide strand may be referred to as “passenger strand”. When processed by the RNAi machinery of the mammalian cell, the passenger strand is degraded and the guide strand is incorporated into the RNA-induced silencing complex (RISC).

According to the embodiment using a double stranded inhibitory RNA, the inventive method can also be phrased as a method of suppressing the expression of a target gene of interest in a mammalian cell, the method comprises: delivering of an inhibitory RNA into the mammalian cell, preferably in an amount sufficient to suppress expression of the target gene of interest; wherein the inhibitory RNA comprises a double stranded nucleotide sequence wherein one strand comprises or consists of a sequence that is complementary to a heterologous RNAi target site, wherein the target gene of interest or a transcript thereof comprises said heterologous RNAi target site; such that the inhibitory RNA produces sequence-specific suppression of expression of the target gene of interest. This embodiment can of course be combined with any other elements, embodiments or aspects of the invention.

In some embodiments, the inhibitory RNA is selected from shRNA, siRNA, miRNA or a combination thereof, preferably shRNAmir. The RNAs can be processed by the RNAi mechanism of a cell, an efficient silencing mechanism and thus lead to high silencing or knock-down capacity.

A shRNAmir is a small hairpin RNA embedded into a microRNA backbone. It can be expressed in a cell from a Polymerase II promoter. The hairpin RNA contains regions of double strand base pairing within the RNA molecule.

The microRNA backbone may comprise mirE, which is a shRNAmir backbone based on the human Micro-RNA 30a (MIR30A). It can be modified for cloning of synthetic shRNAs and micro-RNA optimal processing. The microRNA backbone may comprise mirF. mirF is a derivative of mirE that has been further optimized for optimal micro-RNA processing through removal of a bulge in the basal MIR30A/mire stem.

In particular embodiments, the invention comprises introducing the heterologous RNAi target site into the endogenous locus of the target gene of interest in a mammalian cell, such as on a genomic DNA of the mammalian cell, e.g. using gene editing methods, such as CRISPR/Cas9, zinc finger nucleases, transcription activator-like effector nucleases (TALEN), meganucleases, integration vectors or other methods known in the art, and transducing homozygous clones with a shRNAmir as inhibitory RNA comprising a sequence that is complementary to said target site. Preferably this method uses a tetracycline inducible miR-E expression vector, such that the shRNAmir produces sequence-specific suppression of expression of the target gene of interest.

Preferably the heterologous RNAi target site is in a 5′ untranslated region (UTR), a coding region, in particular an exon, or in a 3′ UTR of the suppression target RNA molecule, wherein 5′ and 3′ refer to the position relative to the RNA sequence of the gene of interest. Preferably, the heterologous RNAi target is in a 5′ UTR, in an exon, or in a 3′ UTR of a gene of interest. Positioning in the 3′ UTR is particularly preferred. Such positions are useful locations for placement of a RNAi target site. It allows expression of the gene of interest without the inhibitory RNA and efficient suppression of expression with the inhibitory RNA.

The heterologous RNAi target site can be placed or inserted in the untranslated regions of messenger RNAs or into noncoding genes. Consequently, for protein coding genes, the coded sequence of target proteins is not altered and the method is amenable to study the loss-of-function (LOF) of protein-coding genes without altering the primary sequence of the corresponding protein. Altogether, the inventive method for LOF is a potent, inducible (hence reversible), off-target free LOF method, that does not change the coding sequence of a gene. As the inventive method is a method optimized for in vitro and in vivo use, the method has wide applications in target identification and validation. In particular preferred embodiments, one or more heterologous RNAi target site are introduced into the transcribed sequence of one or more target genes of interest.

Preferably the heterologous RNAi target site is a non-murine and/or non-human, preferably a non-mammalian nucleic acid sequence. As such, off-target gene suppressions are reduced in humans or mice. In particular embodiments, the heterologous RNAi target site with its size of at least 18 nucleotides in length (or particular sizes as mentioned above) has a non-identity of at least 2, 3, 4, 5, 6, 7, 8, 9, 10, 11, 12, 13, 14, 15, 16, 17 or 18 nucleotides in comparison to any contiguous nucleotide sequence of the size of the heterologous RNAi target site (e.g. at least 18 nucleotides or other sizes of the heterologous RNAi target site as mentioned above) of the genome of the mammalian cell, in particular of the murine or human genome in case of the non-murine and/or non-human nucleic acid sequences.

Preferably the nucleotide sequence of the inhibitory RNA comprises or consists of the nucleic acid sequence 5′-UUCGWWWNNAHHWWCAUCCGGN-3′ (SEQ ID NO: 2), wherein W is A or U, H is A or U or C, and N is A or U or G or C. This nucleic acid sequence is preferably complementary to the heterologous RNAi target site. Such a sequence has the benefit of improved knockdown potency and to overcoming off-target effects in mammalian cells. Also, this sequence is optimized to work and not occur in the two mammalian species Mus musculus and Homo sapiens. In DNA form, e.g. in a DNA suitable for expressing an inhibitory RNA with SEQ ID NO: 2, the sequence can be of SEQ ID NO: 1 with U being replaced by T. The inhibitory RNA may be an interfering RNA.

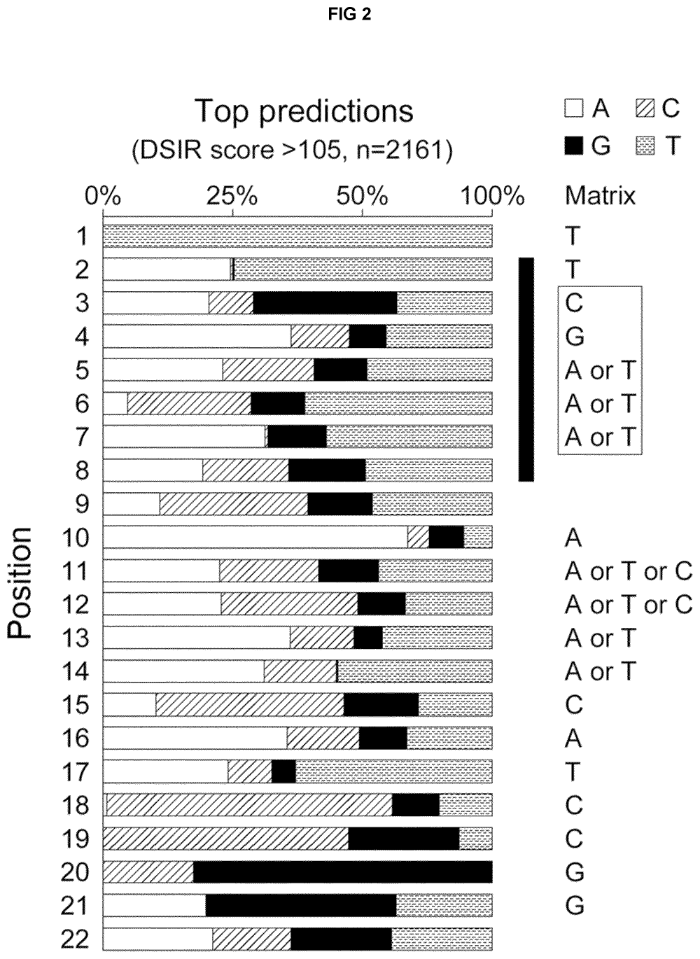

In preferred embodiments of SEQ ID NO: 1 and/or 2, W at position 5 is preferably T/U. In combinable preferred embodiments, W at position 6 is preferably T/U. In combinable preferred embodiments, W at position 7 is preferably A. In combinable preferred embodiments, N at position 8 is preferably A or T/U. In combinable preferred embodiments, N at position 9 is preferably C. In combinable preferred embodiments, H at position 11 is preferably A or T/U. In combinable preferred embodiments, H at position 12 is preferably C. In combinable preferred embodiments, W at position 13 is preferably A. In combinable preferred embodiments, W at position 14 is preferably A. In combinable preferred embodiments, N at position 22 is preferably A, G or T/U. Position here refers to the position corresponding to nucleotides in SEQ ID NO: 1 or 2, e.g. in reference to non-variable nucleotides of SEQ ID NO: 1 or 2, such as at positions 15 to 21. Further preferred nucleotides of the variable nucleotides are weighted as given by the bar size shown in FIG. 2. T in FIG. 2 may be U in RNA. For a heterologous RNAi target site, a complementary nucleic acid to any of these embodiments is preferred.

Preferably the nucleotide sequence of the inhibitory RNA comprises or consists of a nucleic acid sequence selected from any one of SEQ ID NO: 4, 6, 8, 10, 12, 14, 16, 18, 20, 22, 24, or 26 or their encoding DNA sequences of SEQ ID NO: 3, 5, 7, 9, 11, 13, 15, 17, 19, 21, 23, 25 (see Table 1). This nucleic acid sequence is preferably complementary to the heterologous RNAi target site. Particular preferred are sequences TTCGATAACAATATCATCCGGA (SEQ ID NO: 5), TTCGATTAAAACATCATCCGGA (SEQ ID NO: 9), TTCGTATACAATATCATCCGGA (SEQ ID NO: 11) or their respective RNA sequences of SEQ ID NO: 6, 10 and 12. Particularly preferred is a consensus sequence of these 3 sequences, e.g. with regards to SEQ ID NO: 1 or 2, wherein W at position 5 is either T/U or A, A being preferred, wherein W at position 6 is either T/U or A, T/U being preferred, wherein W at position 7 is either T/U or A, A being preferred, wherein N at position 8 is A, wherein N at position 9 is C or A, C being preferred, wherein H at position 11 is A, wherein H at position 12 is T/U or C, T/U being preferred, wherein W at position 13 is A, wherein W at position 14 is T/U, optionally also wherein N at position 22 is A, or any combination thereof. As above, position here refers to the position corresponding to nucleotides in SEQ ID NO: 1 or 2. Such nucleotide sequences of the inhibitory RNA are particularly effective for suppression of expression and reduction of off-target effects. For a heterologous RNAi target site, a complementary nucleic acid to any of these embodiments is preferred. The inhibitory RNA may be an interfering RNA.

In some embodiments, the inhibitory RNA is expressed in the 5′-UTR, coding region or 3′-UTR of a coding gene, e.g. is expressed in the 5′-UTR, coding region or 3′-UTR of a reporter gene, in particular, is expressed in the 3′-UTR of a fluorescent reporter. Such positions are useful locations for placement of the inhibitory RNA. It allows expression of the inhibitory RNA without disturbing expression of the reporter gene.

Preferably the heterologous RNAi target site comprises or consists of the nucleic acid sequence 5′-NCCGGAUGWWDDUNNWWWCGAA-3′, (SEQ ID NO: 28), wherein W is U or A, D is U or A or G, and N is U or A or C or G. Such a sequence has the benefit of improved knockdown potency and to overcoming off-target effects in mammalian cells. A DNA encoding a nucleic acid sequence with the heterologous RNAi target site may comprise SEQ ID NO: 27, with U being replaced by T.

In preferred embodiments of SEQ ID NO: 27 and/or 28, W at position 18 is preferably A. In combinable preferred embodiments, W at position 17 is preferably A. In combinable preferred embodiments, W at position 16 is preferably T/U. In combinable preferred embodiments, N at position 15 is preferably A or T/U. In combinable preferred embodiments, N at position 14 is preferably G. In combinable preferred embodiments, D at position 12 is preferably A or T/U. In combinable preferred embodiments, D at position 11 is preferably G. In combinable preferred embodiments, W at position 10 is preferably T/U. In combinable preferred embodiments, W at position 9 is preferably T/U. In combinable preferred embodiments, N at position 1 is preferably A, C or T/U. Position here refers to the position corresponding to nucleotides in SEQ ID NO: 27 or 28 e.g. in reference to non-variable nucleotides of SEQ ID NO: 27 or 28, such as at positions 2 to 8. Further preferred nucleotides of the variable nucleotides are weighted as given by complementary sequences to the sequences shown in FIG. 2 according to the bar size shown in FIG. 2.

Preferably the heterologous RNAi target site comprises or consists of a nucleic acid sequence selected from any one of SEQ ID NO: 30, 32, 34, 36, 38, or 40. A DNA encoding a nucleic acid sequence with the heterologous RNAi target site may comprise SEQ ID NO: 29, 31, 33, 35, 37, 39, with U being replaced by T. Also, any one of SEQ ID NO: 41-56 are nucleic acids comprising a heterologous RNAi target site of the invention and are part of the invention and can be used in the inventive methods.

In particular preferred are heterologous RNAi target sites selected from TCCGGATGATATTGTTATCGAA (SEQ ID NO: 31), TCCGGATGATGTTTTAATCGAA (SEQ ID NO: 35), TCCGGATGATATTGTATACGAA (SEQ ID NO: 37) or their respective RNA sequences selected from SEQ ID NO: 32, 36 and 38. In particular preferred is a consensus sequence of these 3 sequences, e.g. with regards to SEQ ID NO: 27 or 28, wherein W at position 17 is either T/U or A, T/U being preferred, wherein W at position 16 is either T/U or A, A being preferred, wherein W at position 15 is either T/U or A, T/U being preferred, wherein N at position 15 is T/U, wherein N at position 14 is G or T/U, G being preferred, wherein D at position 12 is T/U, wherein D at position 11 is A or G, A being preferred, wherein W at position 10 is G, wherein W at position 9 is A, optionally also wherein N at position 1 is T, or any combination thereof. As above, position here refers to the position corresponding to nucleotides in SEQ ID NO: 27 or 28. Such heterologous RNAi target sites are particularly effective for suppression of expression and reduction of off-target effects with corresponding inhibitory RNA.

In special embodiments of the invention, the inhibitory RNA and the heterologous RNAi target site (“target site”) comprise sequences selected from (a) to (g)
(a) the inhibitory RNA comprising the sequence transcribed from SEQ ID NO: 1 or the sequence of SEQ ID NO: 2 and the target site comprising the sequence transcribed from SEQ ID NO: 27 or the sequence of SEQ ID NO: 28;
(b) the inhibitory RNA comprising the sequence transcribed from SEQ ID NO: 3 or the sequence of SEQ ID NO: 4 and the target site comprising the sequence transcribed from SEQ ID NO: 29 or the sequence of SEQ ID NO: 30;
(c) the inhibitory RNA comprising the sequence transcribed from SEQ ID NO: 5 or the sequence of SEQ ID NO: 6 and the target site comprising the sequence transcribed from SEQ ID NO: 31 or the sequence of SEQ ID NO: 32;
(d) the inhibitory RNA comprising the sequence transcribed from SEQ ID NO: 7 or the sequence of SEQ ID NO: 8 and the target site comprising the sequence transcribed from SEQ ID NO: 33 or the sequence of SEQ ID NO: 34;
(e) the inhibitory RNA comprising the sequence transcribed from SEQ ID NO: 9 or the sequence of SEQ ID NO: 10 and the target site comprising the sequence transcribed from SEQ ID NO: 35 or the sequence of SEQ ID NO: 36;
(f) the inhibitory RNA comprising the sequence transcribed from SEQ ID NO: 11 or the sequence of SEQ ID NO: 12 and the target site comprising the sequence transcribed from SEQ ID NO: 37 or the sequence of SEQ ID NO: 38;
(g) the inhibitory RNA comprising the sequence transcribed from SEQ ID NO: 13 or the sequence of SEQ ID NO: 14 and the target site comprising the sequence transcribed from SEQ ID NO: 39 or the sequence of SEQ ID NO: 40.

Preferably the mammalian cell lacks a functional genetic copy of the gene of interest in its genome apart from a DNA encoding said suppression target RNA molecule. The genetic copy that shall be non-functional can be an endogenous copy in the mammalian cell. The genetic copy that shall be non-functional may be without the inventive heterologous RNAi target site. Removing functional copies, essentially copies of the gene leading to the expression of the same gene product, e.g. protein or RNA as mentioned below, like non-coding RNA, without the heterologous RNAi target site means that the only copy is on the suppression target-encoding DNA (which can have been provided exogenously), which can be targeted by the inventive method for suppression of expression. This increases sensitivity of functional loss-of-function studies. The presence of the functional genetic copy of the gene/endogenous copy may be possible but is not preferred because then function may have to be studied according to altered gene dose or expression level experiments.

Preferably the gene of interest is an exogenous gene; preferably wherein an endogenous variant of the exogenous gene in the genome of the mammalian cell is disrupted; especially preferred disrupted by CRISPR/Cas. In other particular preferred embodiments, the gene of interest is an endogenous gene, e.g. the gene of interest can be in genomic DNA and/or the DNA encoding said suppression target RNA molecule can be a genomic DNA of the mammalian cell. The coding sequence of the heterologous RNAi target site can be introduced to the endogenous gene, e.g. on a chromosome of the cell, so that the endogenous gene leads to a transcript of the gene operatively linked with the heterologous RNAi target site and thus form the suppression target RNA molecule. Such introduction can be by gene editing or by using an integration vector, which integrates a DNA sequence of the heterologous RNAi target site into the genome and/or into a chromosome of the mammalian cell. In particular embodiments, the endogenous gene can be an oncogene or tumor suppressor gene. Such genes are particularly interesting targets for studying LOF, especially in a drug screening setting.

In preferred embodiments, the gene of interest encodes peptides or proteins, e.g. a reporter gene, such as a gene of a fluorescent protein or an antibiotic resistance gene. In particular preferred embodiments, the one or more heterologous RNAi target sites are in a 3′-UTR of a fluorescent reporter. Reporter genes allow tracking of the inventive nucleic acids.

In particular preferred embodiments, the gene of interest is an oncogene or tumour suppressor gene. Preferably the gene of interest is selected from EGFR, KRAS or STAG1. E.g. an EGFR gene may comprise a sequence that is at least 70%, preferably at least 80%, especially preferred at least 90% or at least 95%, or at least 98%, identical to SEQ ID NO: 53 or SEQ ID NO: 54 and comprises one or more heterologous RNAi target sites. E.g. a KRAS gene may comprise a sequence that is at least 70%, preferably at least 80%, especially preferred at least 90% or at least 95%, or at least 98%, identical to SEQ ID NO: 55 or SEQ ID NO: 56 and comprises one or more heterologous RNAi target sites. E.g. a STAG1 gene may comprise a sequence that is at least 70%, preferably at least 80%, especially preferred at least 90% or at least 95%, or at least 98%, identical to SEQ ID NO: 57 or SEQ ID NO: 58 and comprises one or more heterologous RNAi target sites. Preferably, the one or more heterologous RNAi target sites are in the 3′-UTR of the STAG1 gene.

In other embodiments, the target gene of interest encodes RNAs originating from transcriptional event comprising long non-coding RNAs, microRNAs, rRNAs or tRNAs. Next to proteins such regularity RNA can be expressed or encoded by the gene of interest and thus studied according to the inventive methods.

The invention further provides a nucleic acid comprising the nucleic acid sequence 5′-TTCGWWWNNAHHWWCATCCGGN-3′ (SEQ ID NO: 1), wherein W is A or T, H is A or T or C, and N is A or T or G or C; wherein A is adenine, C is cytosine, G is guanine, T is thymine in DNA or uracil in RNA. T being thymine in DNA or uracil in RNA is according to WIPO ST.26. Such a sequence may be used as complementary nucleotide sequence (to the heterologous RNAi target site) of the inhibitory RNA, or the DNA encoding said inhibitory RNA. Preferred embodiments of these sequences are described above and also constitute preferred embodiments of the nucleic acid provided by the invention.

Preferably, the nucleic acid is RNA, preferably comprising the nucleic acid sequence 5′-UUCGWWWNNAHHWWCAUCCGGN-3′ (SEQ ID NO: 2). Preferred embodiments of these sequences are described above and also constitute preferred embodiments of the nucleic acid provided by the invention.

Preferably, the sequence of SEQ ID NO: 1 is double stranded. As mentioned above, double strands lead to efficient processing of the RNAi machinery and can be used for efficient silencing of gene knock-down. Preferably the nucleic acid comprises a nucleic acid sequence selected from any one of SEQ ID NO: 3, 5, 7, 9, 11, 13, 15, 17, 19, 21, 23, 25.

In particular preferred embodiments, the nucleic acid is RNA and comprises a stem-loop structure, or wherein the nucleic acid is DNA and encodes or transcribes a RNA that comprises a stem-loop structure. Stem-loop structures are RNA structural elements that increase processing by the RNAi machinery and thus lead to improved suppression of expression of an operatively linked gene.

In preferred embodiments, the nucleic acid is RNA and is selected from shRNA, siRNA, miRNA or a combination thereof, preferably shRNAmir, or wherein the nucleic acid is DNA and encodes or transcribes a RNA that is a shRNA, a siRNA, a miRNA or a combination thereof, preferably a shRNAmir. The RNA, especially in form of a shRNAmir, may comprise 5′ and 3′ flanking sequences and loop sequences of a microRNA, especially preferred loop sequences of miR-30 or miR-E.

The invention provides a nucleic acid comprising the nucleic acid sequence 5′-NCCGGATGWWDDTNNWWWCGAA-3′ (SEQ ID NO: 27), wherein W is T or A, D is T or A or G, and N is T or A or C or G. Such a sequence of SEQ ID NO: 27 can be used as heterologous RNAi target site, in particular as DNA encoding a heterologous RNAi target site on a RNA (e.g. as in SEQ ID NO: 28). The nucleic acid may be in a cell, preferably with the nucleic acid sequence of SEQ ID NO: 27 in an endogenous gene. Thus, the cell may express the endogenous gene or transcript thereof comprising a heterologous RNAi target site (SEQ ID NO: 27). Preferred embodiments of these sequences are described above and also constitute preferred embodiments of the nucleic acid provided by the invention. The nucleic acid preferably comprises or consists of a nucleic acid sequence selected from any one of SEQ ID NO: 29, 31, 33, 35, 37, 39,41,43,45,47,49,51,53,55,57.

Provided is a nucleic acid comprising the nucleic acid sequence of SEQ ID NO: 27 and further comprising a gene of interest, e.g. with the sequence of SEQ ID NO: 27 (intended as a heterologous RNAi target site in a transcript of the gene of interest) in a 5′ UTR, coding region, preferably an intron or an exon, or a 3′ UTR of the gene of interest as described above. In preferred embodiments, any of the sequences of the preceding paragraph or described elsewhere herein in connection of a gene with a heterologous RNAi target site is provided according to the invention.

Further provided is a nucleic acid comprising a promoter, an expression sequence and a nucleic acid sequence of a nucleic acid according to the invention. A sequence with a promoter and an expression sequence is also referred to as a gene. The promoter facilitates expression of the expression sequence in a cell or extracellular expression systems. Preferably the sequence of a nucleic acid according to the invention is located in a 5′ UTR of the expression sequence, in the expression sequence or in a 3′ UTR of the expression sequence; and/or preferably wherein the expression sequence is a protein coding sequence, even more preferably an exon. As mentioned above, placement at these locations has benefits with regard to undisturbed expression of the expression sequence, unless expression is suppressed according to the invention.

The invention further provides a mammalian cell comprising a nucleic acid of the invention or a vector of the invention. The invention provides a mammalian cell comprising a DNA encoding a gene of interest operatively linked to a sequence of a heterologous RNAi target site; further comprising a DNA encoding an inhibitory RNA that has a complementary region to the heterologous RNAi target site; wherein said heterologous RNAi target site has a size of at least 18 nucleotides in length. Through the operative linkage, the gene of interest can be suppressed when an inhibitory RNA binds to the heterologous RNAi target site through RNA interference in the mammalian cell.

As described above, also in the mammalian cells, the gene of interest may be an exogenous gene; preferably wherein an endogenous variant of the exogenous gene is disrupted; or an endogenous gene, e.g. wherein the heterologous RNAi target site has been introduced to a genomic gene of interest. The disruption or rendering non-functional of an endogenous variant has been described above with regard to the inventive method and is also an option for the inventive cell.

The invention also provides a method of determining the effects of a loss of function of a gene of interest in a mammalian cell, comprising providing a mammalian cell with a DNA that transcribes a suppression target RNA molecule comprising a heterologous RNAi target site operatively linked to a RNA sequence from the gene of interest;
providing to the cell an inhibitory RNA that has a complementary nucleotide sequence to the heterologous RNAi target site;
wherein binding of the inhibitory RNA to the heterologous RNAi target site suppresses gene expression of the operatively linked RNA sequence from the gene of interest; and wherein said heterologous RNAi target site has a size of at least 18 nucleotides in length;
and observing a change of phenotype of the mammalian cell between the mammalian cells with the provided inhibitory RNA and without providing the inhibitory RNA.

Preferably providing to the cell an inhibitory RNA comprises suppressing gene expression according to the inventive methods as described above.

In particular preferred the invention is for genetic target validation (testing genes of interest as drug targets), loss-of-function studies of genes of interest as well as phenotype establishment to guide drug development in cells, especially in cancer cells.

The invention also provides a method of producing a knockdown mutant of a mammalian cell, the method comprises:
delivering of an inhibitory RNA into the mammalian cell in an amount sufficient to suppress expression of a target gene,
wherein the inhibitory RNA molecule comprises a double stranded region comprising a sequence that is complementary to a heterologous RNAi target site, wherein a target gene of interest or a transcript thereof comprises said heterologous RNAi target site,
such that the inhibitory RNA molecule produces sequence-specific suppression of expression of the target gene of interest, thereby producing the knockdown mutant. Embodiments and preferred aspects of the invention as described above also relate to this method of the invention.

Preferably, the present invention is defined according to the following numbered embodiments. These numbered embodiments can be combined with any further aspects and embodiments of this description.

1. A method of suppressing gene expression, comprising providing a mammalian cell with a suppression target RNA molecule comprising a heterologous RNAi target site operatively linked to a RNA sequence from a gene of interest, or with a DNA encoding said suppression target RNA molecule;
providing to the cell an inhibitory RNA that has a complementary nucleotide sequence to the heterologous RNAi target site;
wherein binding of the inhibitory RNA to the heterologous RNAi target site suppresses gene expression of the operatively linked RNA sequence from the gene of interest; and wherein said heterologous RNAi target site has a size of at least 18 nucleotides in length.

2. The method of embodiment 1, wherein the DNA encoding said suppression target RNA molecule is an expression vector or a genomic DNA of the mammalian cell.

3. The method of embodiment 1 or 2, wherein the inhibitory RNA is expressed from a DNA encoding said inhibitory RNA in said mammalian cell.

4. The method of embodiment 3, wherein expression of the inhibitory RNA is inducible expression by an inducible promoter, preferably wherein the DNA encoding said inhibitory RNA comprises a tetracycline-responsive element promoter.

5. The method of any one of embodiments 1 to 4, wherein the inhibitory RNA is selected from shRNA, siRNA, miRNA or a combination thereof, preferably shRNAmir.

6. The method of any one of embodiments 1 to 5, wherein the inhibitory RNA comprises a double strand, wherein one strand of the double strand is complementary to the heterologous RNAi target site.

7. The method of any one of embodiments 1 to 6, wherein the heterologous RNAi target site is in a 5′ untranslated region (UTR), in a coding region, preferably an exon, or in a 3′ untranslated region (UTR) of the suppression target RNA molecule, preferably in a 3′ UTR of the suppression target RNA molecule, wherein 5′ and 3′ refer to the position relative to the RNA sequence of the gene of interest.

8. The method of any one of embodiments 1 to 7, wherein the heterologous RNAi target site is a non-murine and/or non-human, preferably a non-mammalian nucleic acid sequence.

9. The method of any one of embodiments 1 to 8, wherein the nucleotide sequence of the inhibitory RNA that is complementary to the heterologous RNAi target site comprises or consists of the nucleic acid sequence 5′-UUCGWWWNNAHHWWCAUCCGGN-3′ (SEQ ID NO: 2), wherein W is A or U, H is A or U or C, and N is A or U or G or C.

10. The method of any one of embodiments 1 to 9, wherein the nucleotide sequence of the inhibitory RNA that is complementary to the heterologous RNAi target site comprises or consists of a nucleic acid sequence selected from any one of SEQ ID NO: 4, 6, 8, 10, 12, 14, 16, 18, 20, 22, 24, or 26.

11. The method of embodiment 10, wherein the nucleotide sequence of the inhibitory RNA comprises or consists of a nucleic acid sequence selected from any one of SEQ ID NO: 6, 10 and 12.

12. The method of any one of embodiments 1 to 11, wherein the heterologous RNAi target site comprises or consists of the nucleic acid sequence 5′-NCCGGAUGWWDDUNNWWWCGAA-3′, (SEQ ID NO: 28), wherein W is U or A, D is U or A or G, and N is U or A or C or G.

13. The method of any one of embodiments 1 to 12, wherein the heterologous RNAi target site comprises or consists of a nucleic acid sequence selected from any one of SEQ ID NO: 30, 32, 34, 36, 38, or 40.

14. The method of embodiment 13, wherein the heterologous RNAi target site comprises or consists of a nucleic acid sequence selected from any one of SEQ ID NO: 32, 36 and 38.

15. The method of any one of embodiments 1 to 14, wherein the mammalian cell lacks a functional genetic copy of the gene of interest in its genome apart from a DNA encoding said suppression target RNA molecule.

16. The method of any one of embodiments 1 to 15, wherein the gene of interest is on an exogenous gene; preferably wherein an endogenous variant of the exogenous gene in the genome of the mammalian cell is disrupted; especially preferred disrupted by CRISPR/Cas.

17. The method of any one of embodiments 1 to 16, wherein the gene of interest is EGFR, KRAS or STAG1; or wherein the gene of interest is an oncogene or tumor suppressor gene.

18. The method of any one of embodiments 1 to 15, wherein the gene of interest is an endogenous gene, preferably an oncogene or tumor suppressor gene, preferably EGFR, KRAS or STAG1.

19. A nucleic acid comprising the nucleic acid sequence 5′-TTCGWWWNNAHHWWCATCCGGN-3′ (SEQ ID NO: 1), wherein W is A or T, H is A or T or C, and N is A or T or G or C; wherein A is adenine, C is cytosine, G is guanine, T is thymine in DNA or uracil in RNA.

20. The nucleic acid of embodiment 19 being RNA, preferably comprising the nucleic acid sequence 5′-UUCGWWWNNAHHWWCAUCCGGN-3′ (SEQ ID NO: 2).

21. The nucleic acid of embodiment 19 or 20, wherein the sequence of SEQ ID NO: 1 is double stranded.

22. The nucleic acid of any one of embodiments 19 to 21, comprising a nucleic acid sequence selected from any one of SEQ ID NO: 3, 5, 7, 9, 11, 13, 15, 17, 19, 21, 23, 25.

23. The nucleic acid of embodiment 22, wherein the nucleic acid sequence is selected from any one of SEQ ID NO: 5, 9, 11.

24. The nucleic acid of any one of embodiments 19 to 23, wherein the nucleic acid is RNA and comprises a stem-loop structure, or wherein the nucleic acid is DNA and encodes or transcribes a RNA that comprises a stem-loop structure.

25. The nucleic acid of any one of embodiments 19 to 24, wherein the nucleic acid is RNA and is selected from shRNA, siRNA, miRNA or a combination thereof, preferably shRNAmir, or wherein the nucleic acid is DNA and encodes or transcribes a RNA that is a shRNA, a siRNA, a miRNA or a combination thereof, preferably a shRNAmir.

26. A nucleic acid comprising the nucleic acid sequence 5′-NCCGGATGWWDDTNNWWWCGAA-3′ (SEQ ID NO: 27), wherein W is T or A, D is T or A or G, and N is T or A or C or G.

27. The nucleic acid of embodiment 26, comprising or consisting of a nucleic acid sequence selected from any one of SEQ ID NO: 29, 31, 33, 35, 37, 39, 41, 43, 45, 47, 49, 51, 53, 55, 57.

28. The nucleic acid of embodiment 27, wherein the nucleic acid sequence is selected from any one of SEQ ID NO: 31, 35, 37.

29. A nucleic acid comprising a promoter, an expression sequence and a nucleic acid sequence of a nucleic acid according to any one of embodiments 19 to 28; preferably wherein the sequence of a nucleic acid according to any one of embodiments 19 to 28 is located in a 5′ UTR of the expression sequence, in the expression sequence or in a 3′ UTR of the expression sequence; and/or preferably wherein the expression sequence is a protein coding sequence; and/or preferably wherein the sequence of a nucleic acid according to any one of embodiments 19 to 28 is located in a coding region, preferably an exon, of the expression sequence.

30. A vector comprising a nucleic acid of any one of embodiments 19 to 29 as DNA.

31. A mammalian cell comprising a DNA encoding a gene of interest operatively linked to a sequence of a heterologous RNAi target site;
further comprising a DNA encoding an inhibitory RNA that has a complementary region to the heterologous RNAi target site;
wherein said heterologous RNAi target site has a size of at least 18 nucleotides in length.

32. The cell of embodiment 31, wherein the gene of interest is on an exogenous gene; preferably wherein an endogenous variant of the exogenous gene is disrupted.

33. A mammalian cell comprising a nucleic acid of any one of embodiments 19 to 29 or a vector of embodiment 30.

34. A method of determining the effects of a loss of function of a gene of interest in a mammalian cell, comprising
providing a mammalian cell with a DNA that transcribes a suppression target RNA molecule comprising a heterologous RNAi target site operatively linked to a RNA sequence from the gene of interest;
providing to the cell an inhibitory RNA that has a complementary nucleotide sequence to the heterologous RNAi target site;
wherein binding of the inhibitory RNA to the heterologous RNAi target site suppresses gene expression of the operatively linked RNA sequence from the gene of interest; and wherein said heterologous RNAi target site has a size of at least 18 nucleotides in length;
and observing a change of phenotype of the mammalian cell between the mammalian cells with the provided inhibitory RNA and without providing the inhibitory RNA; preferably wherein providing to the cell an inhibitory RNA comprises suppressing gene expression according to any one of embodiments 1 to 18.

Throughout the present disclosure, the articles “a”, “an”, and “the” are used herein to refer to one or to more than one (i.e., to at least one) of the grammatical object of the article.

As used herein, words of approximation such as, without limitation, “about”, “substantial” or “substantially” refer to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by e.g. ±10%.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The “comprising” expressions when used on an element in combination with a numerical range of a certain value of that element means that the element is limited to that range while “comprising” still relates to the optional presence of other elements. E.g. the element with a range may be subject to an implicit proviso excluding the presence of that element in an amount outside of that range. As used herein, the phrase “consisting essentially of” requires the specified integer(s) or steps as well as those that do not materially affect the character or function of the claimed invention. As used herein, the closed term “consisting” is used to indicate the presence of the recited elements only.

### EXAMPLES

The invention is further described by way of the following examples as embodiments of the invention without being limited thereto.

### Example 1: Rational Design and Selection of RNA Antisense Sequences of the Invention Using siRNA Predictions

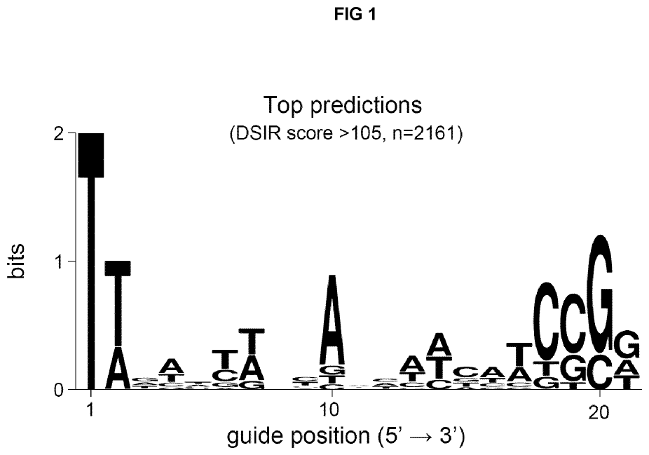

To design pairs of artificial shRNAs and matching target sites that trigger effective and selective target suppression with minimal off-target effects, the nucleotide composition of shRNAs target sites that reach exceptionally high performance scores were analyzed in a well-established siRNA prediction tool (DSIR=Designer of Small Interfering RNA7) and contain no A or T in position 20 to eliminate shRNAmirs that produce RISC-loadable small RNAs from the passenger strand (8). Top scoring predictions (DSIR score>105, n=2161) displayed prominent nucleotide biases, particularly for TT in positions 1-2, for A in position 10, and for CCG in positions 18-20 (FIG. 1, 2), which are in line with established requirements for efficient RISC loading and target mRNA cleavage. Based on strong biases at the 5′-end, the first two nucleotides of guides were fixed to TT.

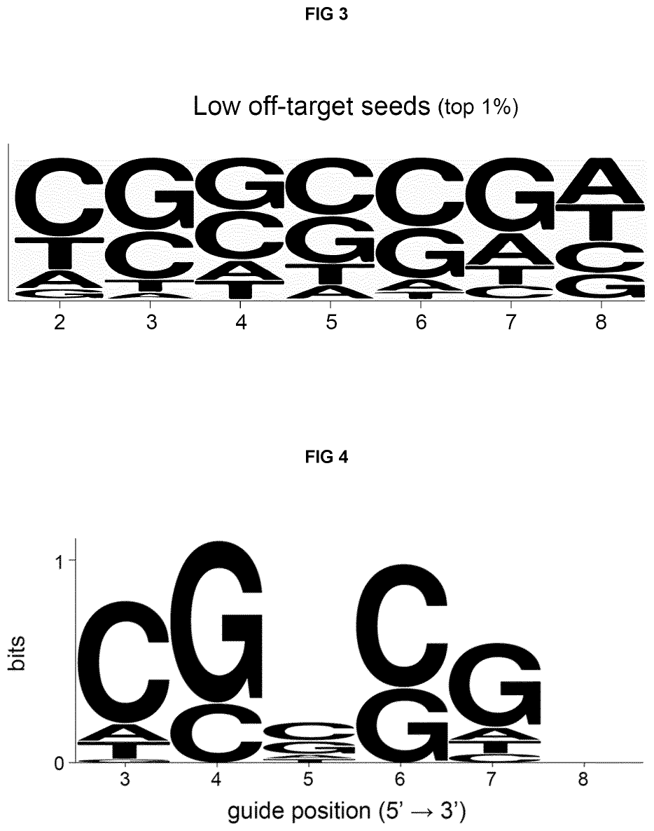

Next, possible off-target effects were minimized using siSPOTR, an siRNA-based prediction tool that assesses off-target potential of different siRNA seed sequences (guide strand positions 2-8) in the human and mouse genome. Seed sequences with the lowest predicted off-target activity (top 1%) showed biases for C and/or G in guide positions 2-6 (FIG. 3), but 23% contained a T at the 5′ end of the seed sequence that is required in our design. Among these, particularly strong biases for CG were observed in the following two positions, i.e. positions 2 and 3 (FIG. 4), which were found in 73% of all seed sequences harboring a 5′ T. Overall, 17% of all top-scoring seed sequences harbored TCG at their 5′ end, making it the second most common triplet (after CGC, which was found in 20%). Based on these analyses, the first 4 nucleotides of the guide were fixed to TTCG (FIG. 2). For the following positions (following the first four nucleotides), it was assumed, that introducing additional GC biases would destroy 5′-3′ asymmetry of small RNA duplexes that is critically required for efficient RISC loading. To maintain sufficient asymmetry, the next 3 positions were biased towards A or T, which in most positions is in alignment with nucleotide biases associated with knockdown efficacy.

For the remaining sequence 3′ of the seed region, nucleotide features were selected according to knockdown efficacy based on our DSIR analysis, which are remarkably prominent and cannot all be explained through established processing requirements (FIG. 2). Altogether, this established a 22-nt matrix for the design of ARTi-shRNAmirs (TTCGWWWNNAHHWWCATCCGGN (SEQ ID NO: 1); W=A/T, H=A/T/C; N=A/T/G/C). In a last step, possible off-target effects were further reduced by eliminating all guides whose extended seed sequence (guide positions 2-14) had a perfect match in the human or mouse transcriptome and, finally, selected six top-scoring ARTi predictions for experimental validation.

### Example 2: Cloning and Expression of miRNA-Based shRNAs (shRNAmirs) Using miR-E Expression Plasmids

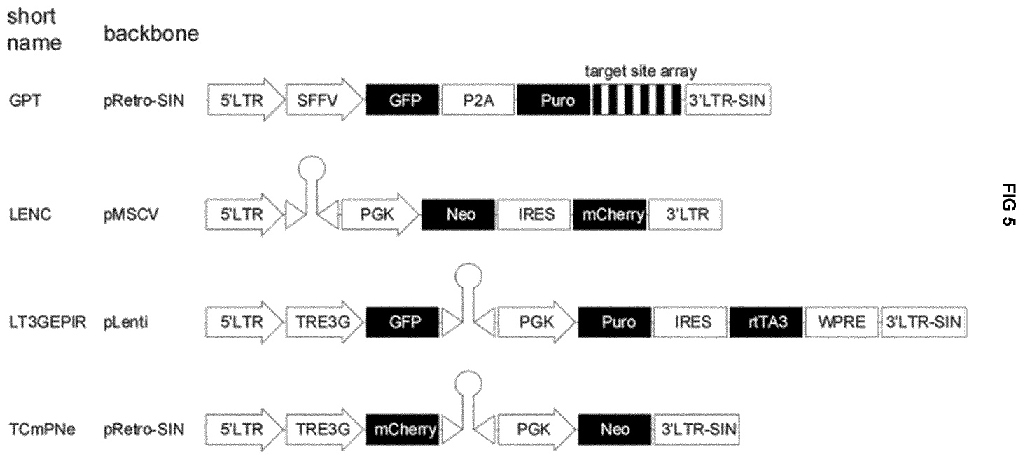

ARTi-shRNAs were ordered as single stranded DNA oligonucleotides, amplified by PCR and cloned into the following retroviral or lentiviral miR-E improved shRNAmir expression plasmids using EcoRI, Xhol restriction digest and gibson assembly: pSin-TRE3G-mCherry-miR-E-PGK-Neo (TCmPNe; reporter assay and competitive proliferation assay in RTT-MEFs) or modified versions of pMSCV-miR-E-PGK-Neo-IRES-mCherry (LENC; Addgene plasmid #111163) and pRRL-TRE3G-GFP-mir-E-PGK-Puro-IRES-rtTA3 backbone (LT3GFPIR, Addgene plasmid #111177) (FIG. 5).

The GPT sequence (SFFV-GFP-P2A-Puro-ARTi-target sensor) was cloned into pRSF91 retroviral plasmid (Galla et al. 2011) using gibson assembly (FIG. 5).

### Example 2.1: Reporter Assay of GFP Knockdown Efficiency of shRNAmirs in Immortalized MEFs

Six ARTi-shRNA sequences as well as validated shRNAs were cloned into an inducible miR-E expression vector, resulting in ARTi-shRNAmir constructs and subsequently transduced into immortalized mouse embryonic fibroblasts (MEFs), containing a stable integration of the Tet-On Advanced transactivator (Rosa26-rtTA-M2 (RRT)-MEFs) in the Rosa26 locus and expressing a GFP reporter harboring the respective ARTi-shRNAmir target sites in its 3′-UTR. This “GFP-sensor assay” allows for the quantification of knockdown levels by reading out the GFP fluorescence which is impacted by the shRNA-mediated knockdown of the mRNA containing the shRNA target sequences in its 3′-UTR. For each reporter cell line single cells were FACS-sorted into 96-well plates using a FACSAria III cell sorter (BD Bioscience) to obtain single-cell derived clones. These clones were transduced with retroviruses expressing either the respective doxycycline inducible ARTi shRNA or validated shRNAs and mCherry fluorescence marker. ShRNA expression was induced with doxycycline and GFP levels were quantified via flow cytometry 2 days post induction. Knockdown efficiency was calculated as 1 minus the ratio of mean GFP signal in mCherry+(shRNA+) cells over mCherry-cells and normalized to Renilla luciferase specific neutral control shRNA (Ren713).

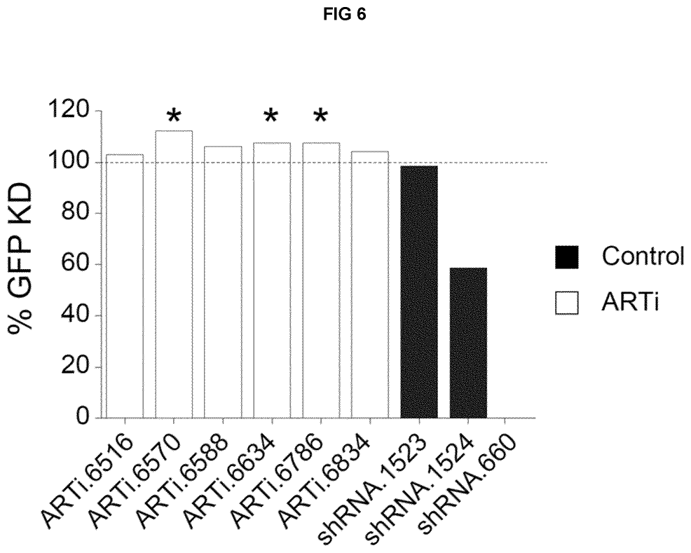

Knockdown (KD) efficacy was evaluated via flow cytometry-based quantification of GFP fluorescence levels in cells expressing ARTi-shRNAmirs compared to cells expressing validated shRNAmirs or no shRNAmir. After induction of shRNA expression, all ARTi-shRNAmirs showed strong and durable knockdown of GFP with effect sizes matching or exceeding those of strong validated shRNAs (FIG. 6).

### Example 2.2: Competitive Proliferation Assay of 3T3, EPP2 and RN2 Cells Expressing shRNAmirs and Toxicity of shRNAmirs

The 3 best-performing ARTi-shRNAmirs were analyzed for their effects on proliferation of diverse mouse cell lines originating from different tissues. This experimental step monitors potential off-target effects. Should an off-target, relevant to the proliferative capacity be knocked down, a decrease in cellular fitness would be expected. NIH 3T3 (embryonic fibroblasts), EPP2 (PDAC) and RN2 (AML) cells were transduced with ARTi-shRNAs or validated control shRNAs. Subsequently, the percentage of shRNA expressing cells was monitored via flow cytometry in regular intervals for 8 days in competitive co-culture assays. For competitive proliferation assays, NIH 3T3, EPP2 and RN2 cells were retrovirally transduced with shRNAmir expression plasmids and initial infection levels were determined by flow cytometry based on mCherry expression 4 days post transduction (Day 0). The percentage of infected cells was then further monitored in regular intervals to determine the effects of shRNA expression in the absence of an endogenous target gene for the ARTi-shRNAmirs.

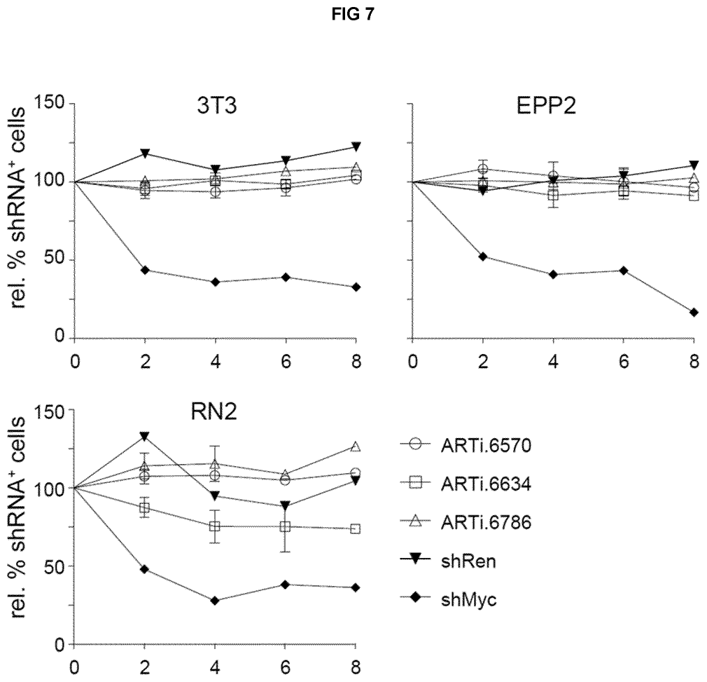

As expected, cells expressing an shRNA targeting the essential transcription factor Myc rapidly depleted from the cultures, while the percentage of cells expressing the neutral Renilla luciferase control shRNA stayed unchanged over time. Similarly, no detectable toxicity was observed for the three tested ARTi shRNAs in NIH 3T3 and EPP2 cells. In RN2 cells, ARTi-shRNA 6634, but not the other ARTi-shRNAs caused a growth disadvantage compared to non-transduced cells (FIG. 7).

### Example 2.3: Transcriptional Profiling in NIH 3T3, EPP2 and MR125 Cells Expressing shRNAmirs and Number of Downregulated Genes

To directly quantify off-target effects of the top 3 ARTi-shRNAs, transcriptome profiling in NIH 3T3, EPP2 and MR125 cells expressing ARTi-shRNAs or empty vector control was performed. For the unbiased identification of ARTi shRNA off-targets NIH-3T3, EPP2 and MR125 cells were retrovirally transduced with shRNAmir expression plasmids and selected for shRNA expression using G418 Sulfate (0.5 mg/mL for EPP2 and NIH 3T3 and 1 mg/mL for MR125, respectively; Gibco). 1.5×106 (NIH 3T3 and EPP2) or 2×106 (MR125) cells were pelleted, washed with PBS, lysed, treated with DNAse I and whole RNA was extracted using magnetic beads (in-house) and a KingFisher Duo Prime Purification System (ThermoFisher). Next generation sequencing libraries were prepared using QuantSeq 3′ mRNA-Seq Library Prep Kit (Lexogen) according to manufacturer's instructions and sequenced on a HiSeq2500 platform (Illumina).

RefSeq genes and 3′-UTR annotations were prepared from the UCSC table browser (https://genome.ucsc.edu/cgi-bin/hgTables, May 2016). For any RefSeq genes lacking an annotated 3′-UTR, Ensembl v84 3′-UTRs were checked for corresponding entries and added if available, resulting in a total of 33,163 annotated 3′-UTRs for 22,552 genes. Reads were aligned to the GRCm38 primary assembly using Slamdunk v0.2.410 in QuantSeq mode (slamdunk map-5 12-n 100-q) and alignments were filtered to recover multimappers with the acquired 3′-UTR set (slamdunk filter-b). Reads were quantified against said 3′-UTR annotation using featureCounts v1.5.0-p211 using stranded counting and counting multimapping reads (−M ˜s 1). Principal component analysis based on the 100 most highly variable genes was performed with DeSeq2. The number of downregulated genes were calculated by comparing TPM normalized reads for each ARTi shRNA to empty vector control and filtering for genes with a fold-change 2 2 and TPM≥10.

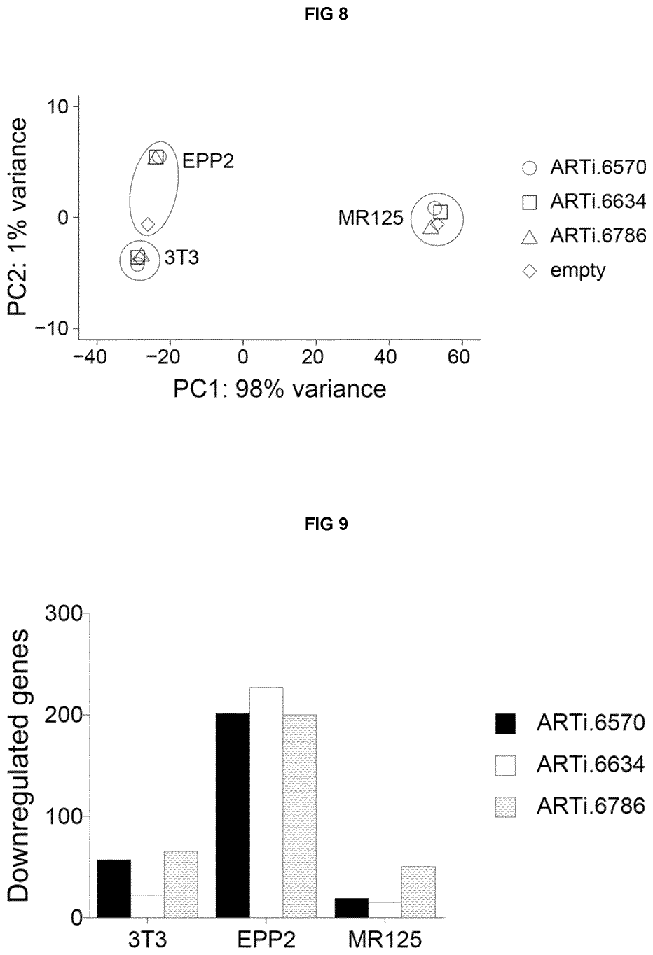

Principal component analysis showed that the samples clustered by tissue (adherent versus suspension cells; PC1; 98% of variance) rather than shRNA, indicating that the expression of ARTi-shRNAs did not have strong transcriptional effects, even in the absence of target gene expression (FIG. 8). Furthermore, also the number of deregulated genes was determined mainly by cell line and not by shRNA expression, with all ARTi-shRNAs showing comparable numbers of downregulated genes per cell line (FIG. 9). ARTi-shRNA 6634 was consequently deprioritized for adverse effect on RN2 cells and ARTi-shRNAs 6570 and 6786 were selected as they showed no considerable toxicity and only mild transcriptional responses in all tested cell lines, as standard reagents for future applications.

### Example 3: Validation of Novel Methods for RNA Interference In Vitro and In Vivo

### Example 3.1: Genome Engineering of EGFR and Ectopically Expression of EGFRdel19::ARTi in PC-9 Cells

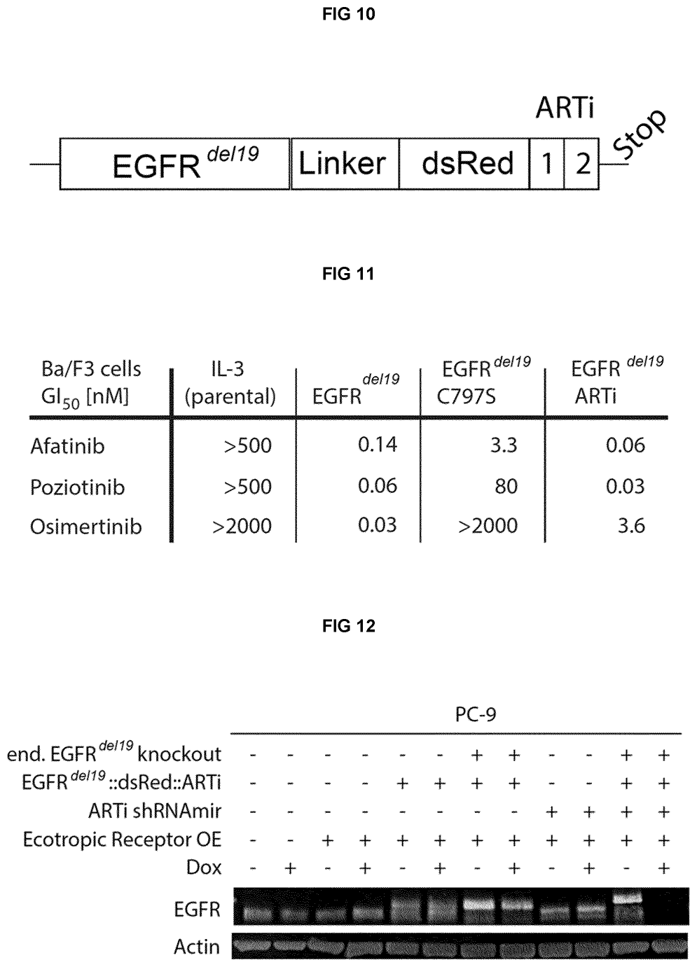

The ARTi method was validated for EGFR, an established oncology target. Aberrant signaling of EGFR is associated with the initiation and maintenance of lung cancer. Oncogenic variants of EGFR, including the deletion of exon 19 (EGFR^{del19}), are characterized by chronically increasing signaling and thereby sustaining the growth and replicative potential of tumor cells. Tyrosine kinase inhibitors (TKIs) targeting EGFR have been proven efficacious in inducing tumor regressions and increasing overall survival of EGFR mutant lung cancer patients. Therefore, EGFR is a well-established oncogene, suitable for validating the ARTi approach. To this end, the ARTi-shRNAmir target sequences were inserted into a construct designed to drive the expression of an oncogenic version of EGFR (EGFR^{del19}::Linker::dsRed::ARTi; Note: the ARTi sequence is part of the open reading frame and thus an in-frame fusion) (FIG. 10).

### Example 3.2: Functional Validation of EGFRdel19::ARTi Construct in Ba/F3 Cells (Compound Treatment)

The functionality of this construct was validated by transforming Ba/F3 cells. The murine Ba/F3 haematopoetic cells are dependent on exogenously provided interleukin (IL)-3. Upon the expression of activated human oncogenes, Ba/F3 cells can be maintained in the absence of IL-3 due to the oncogenic and proliferative stimulus provided by the functional oncogene. Ba/F3 cells were transduced with an ecotropic pMSCV-EGFR^{del19}::V5::dsRed::ARTi-PGK-Blasticidin retrovirus cloned at GenScript, China and produced in Platinum E cells (Cell Biolabs) in the presence of 4 μg/mL Polybrene (Merck Millipore). After 72 hours, stable transgenic cells were selected by using 50 μg/mL Blasticidin (SIGMA, P9620) without adding IL-3.

The EGFR^{del19}::Linker::dsRed::ARTi construct was able to confer IL-3 independent replicative potential to Ba/F3 cells indicating that the construct is functional. Consistently, EGFR^{del19}::Linker::dsRed::ARTi dependent cells were sensitive to EGFR selective TKIs further supporting the notion that the construct is functional (FIG. 11).

### Example 3.3: Western Blot Analysis Confirming Doxycycline Induced Knockdown of EGFRdel19::ARTi

The EGFR^{del19}::Linker::dsRed::ARTi construct was transduced into EGFR^{del19}dependent PC-9 cells. The endogenous EGFR gene was then knocked out using CRISPR/Cas9, making the PC-9 cells dependent on EGFR^{del19}::dsRed::ARTi. PC-9 cell pellets for different genetic backgrounds and treatments were lysed in triton lysis buffer, sonicated, and stored at −80° C. until for protein detection, the pellets were thawed on ice, followed by 15 minutes centrifugation at 13,000 rpm at 4° C. Then, cell lysates were loaded onto a 12% pre-casted SDS-polyacrylamide gel electrophoresis and proteins were transferred onto a nitrocellulose membrane and PDVF-membrane, respectively. Both membranes were probed with the respective primary antibodies overnight. The next day, the secondary antibodies conjugated with fluorescent dye were added and the proteins were detected by the Odyssey detection system.

Doxycycline induced expression of ARTi-shRNAmirs (ARTi #6570) resulted in a reduction of EGFR^{del19}::dsRed::ARTi protein levels, demonstrated by Western blotting (FIG. 12).

### Example 3.4: Proliferation Assay and Subsequent Crystal Violet Staining of Parental and Engineered PC-9 Cells in Absence or Presence of Doxycycline

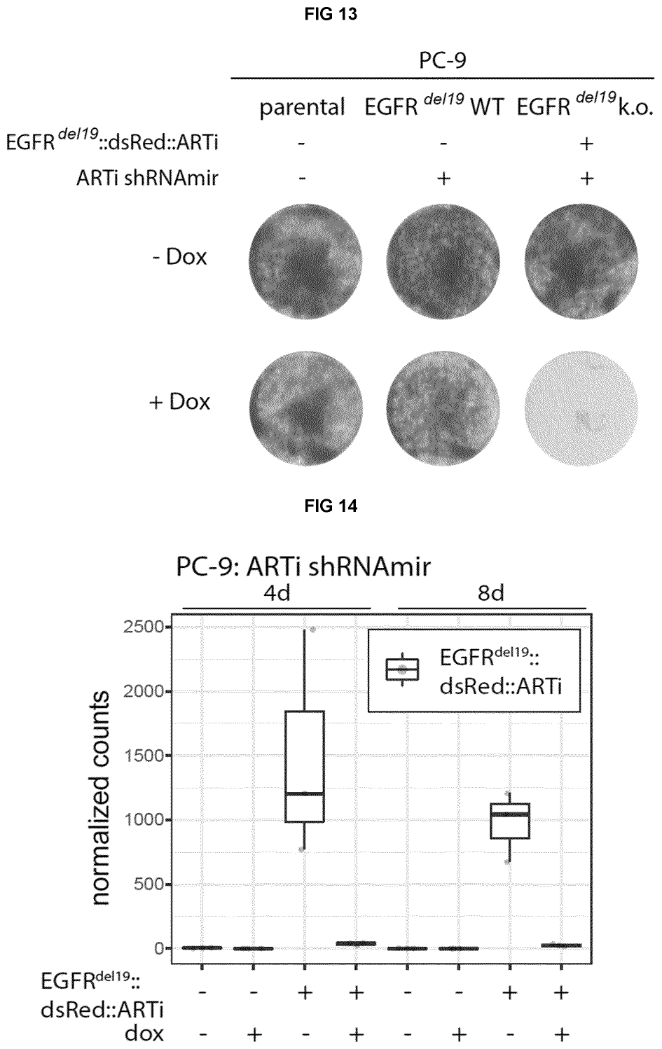

To assess the strength of the loss-of-function phenotype associated with the ARTi mediated knock-down of EGFR in vitro, crystal violet staining assays (FIG. 13) were performed. 15 000 PC-9 cells/well (parental, EGFR^{del19} wild type and EGFR^{del19}knockout) were plated in a 6-well plate containing 2 mL tetracycline-free medium with or without 1 μg/mL doxycyline. Medium was exchanged every 2-3 days. After 9 days, the wells were washed with cold 1×PBS and subsequently stained with 1 mL of 2.3% crystal violet solution for 10 minutes. Then, the wells were washed with ultrapure water and dried overnight.

Doxycycline induced expression of ARTi-shRNAmirs resulted in a reduced proliferative capacity and cell survival of EGFR^{del19}::dsRed::ARTi dependent cell lines but not their wild type counterparts (FIG. 13).

### Example 3.5: Validation of ARTi Knockdown Efficiency by RNA Sequencing

To monitor the extend of target gene modulation and the knockdown of the EGFR^{del19}::dsRed::ARTi transgene, an RNA-seq experiment was conducted. Engineered PC-9 cells were cultured in the presence of 1 μg/ml doxycycline to induce expression of the ARTi-shRNAmir. Media containing doxycycline was replenished twice per week and on day 4 and day 8 after initial treatment, 2×10⁶ doxycycline-treated or untreated control cells were harvested, washed with PBS, lysed, treated with DNAse I (QIAGEN) and total RNA was extracted using the RNeasy Mini Kit (QIAGEN). Next generation sequencing libraries were prepared using the QuantSeq 3′ mRNA-Seq Library Prep Kit (Lexogen) according to the manufacturer's instructions and sequenced on an Illumina platform.

While counts mapping to the human codon optimized EGFR^{del19}::dsRed::ARTi transgene were not detected in PC-9 cells expressing the ARTi-shRNAmir, the construct was readily detected in non-doxycycline treated PC-9 cells transduced with the construct. Addition of doxycycline resulted in an almost complete drop in mappable reads, consistent with a strong knockdown, presumably by mRNA cleavage (FIG. 14).

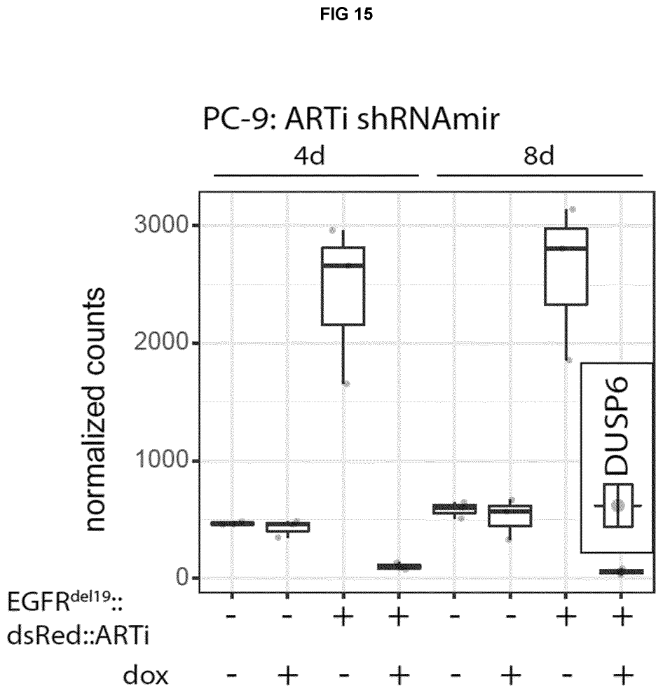

Consistent with the near complete knockdown of the EGFR^{del19}::dsRed::ARTi transgene, strong modulation of the mitogen activated kinase (MAPK) pathway downstream gene DUSP6 was observed (FIG. 15). In parental cells DUSP6 expression was detected at basal levels at day 4 and day 8 of sampling. Upon overexpression of EGFR^{del19}::dsRed::ARTi and knockout of endogenous EGFR, DUSP 6 levels were induced, suggesting that the transgene activates MAPK signaling, exceeding the basal levels. ARTi-mediated knockdown of EGFR^{del19}::dsRed::ARTi resulted in a near complete decrease of DUSP6 levels, below the basal expression. These data suggest that ARTi-mediated loss of EGFR function is associated with a strong inhibition of the MAPK pathway in PC-9 cells.

### Example 3.6: In Vivo Experiment Comparing Doxycycline Induced EGFRdel19-ARTi Knockdown to Osimertinib-Mediated Inhibition of EGFRdel19 Oncogenic Signaling

Subsequently, the ARTi method was tested in vivo. PC-9 cells expressing the ARTi-shRNAmir construct were injected into mice. Female BomTac:NMRI-Foxn1nu mice were obtained from Taconic Denmark at an age of 6-8 weeks. After arrival of the local animal facility at Boehringer Ingelheim RCV GmbH & Co KG mice were allowed to adjust to housing conditions at least for 5 days before the start of the experiment. Mice were group-housed under pathogen-free and controlled environmental conditions and handled according to the institutional, governmental and European Union guidelines (Austrian Animal Protection Laws, GV-SOLAS and FELASA guidelines). Animal studies were approved by the internal ethics committee and the local governmental committee. To establish subcutaneous tumors mice were injected with 1×10⁷ PC-9 cells in PBS with 5% FCS. Tumor diameters were measured with a caliper three times a week. The volume of each tumor [in mm³]was calculated according to the formula “tumor volume=length*diameter²*π/6.” To monitor side effects of treatment, mice were inspected daily for abnormalities and body weight was determined three times per week. Animals were sacrificed when the tumors reached a size of 1,500 mm³. Mice were dispatched randomly into treatment groups then the tumor size was ˜130 and 190 mm³. 2 mg/ml doxycycline and 5 mg/ml sucrose were added to the drinking water of the treatment groups, the control group received water with 5 mg/ml sucrose only. Osimertinib (Tagrisso, AstraZeneca) was dosed per os daily at a dose of 25 mg/kg and control mice were dosed with Natrosol.

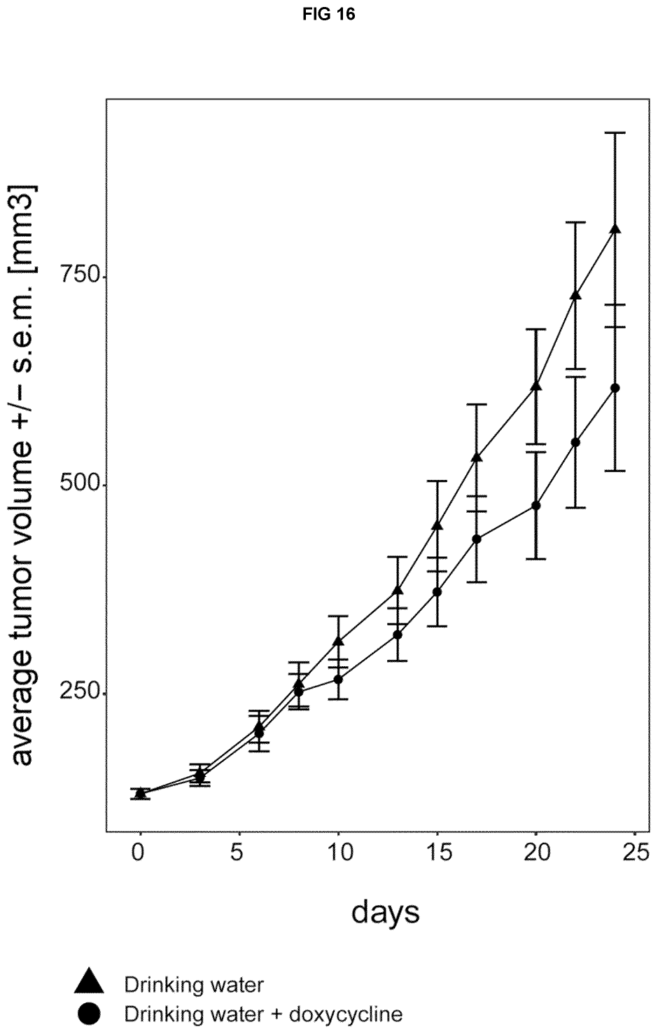

When treated with doxycycline, similar tumor growth in both groups was observed, suggesting that neither doxycycline, nor the expression of the ARTi-shRNAmir interfered with the in vivo growth of PC-9 cells in this xenotransplantation experiment (FIG. 16).

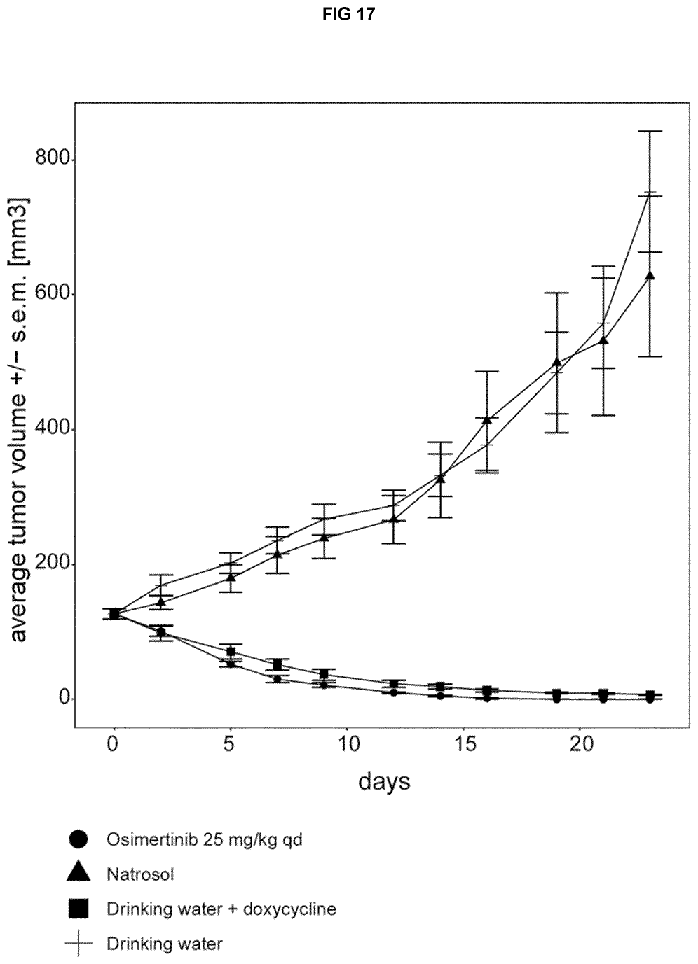

Next, PC-9 cells were injected in which endogenous EGFR alleles were knocked out and rescued by the expression of the EGFR-ARTi construct (FIG. 10). In addition, these cells contain a doxycycline inducible ARTi-shRNAmir construct. Upon doxycycline treatment, established tumors went into regression, showing that the treatment abrogated EGFR signaling. ARTi-mediated knockdown of EGFR in vivo phenocopied the pharmacological inhibition of EGFR using Osimertinib at a clinically relevant dose (FIG. 17). These data suggest that ARTi-mediated loss-of-function induces phenotypes similar to an established inhibitor.

### Example 4: Establishment of an In Vivo Benchmark Phenotype Through Oncogene Switching

### Example 4.1: Genome Engineering of KRASG12R Dependent MIA PaCa2 Cells

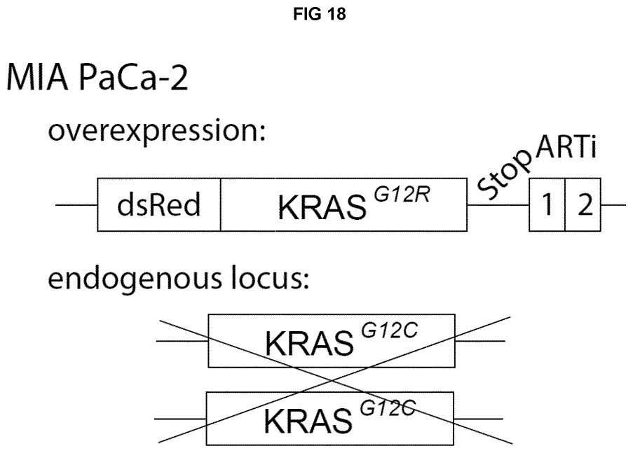

KRAS^{G12C} dependent MIA PaCa-2 cells were engineered such that they express the ARTi-shRNAmir under a doxycycline inducible promoter and a dsRed tagged version of KRAS^{G12R}. Subsequently, endogenous KRAS was knocked out using CRISPR/Cas9 to make the cells dependent on KRAS^{G12R}, thus switching the oncogene (FIG. 18).

### Example 4.2: Validating the Knockdown Efficiency for KRASG12R

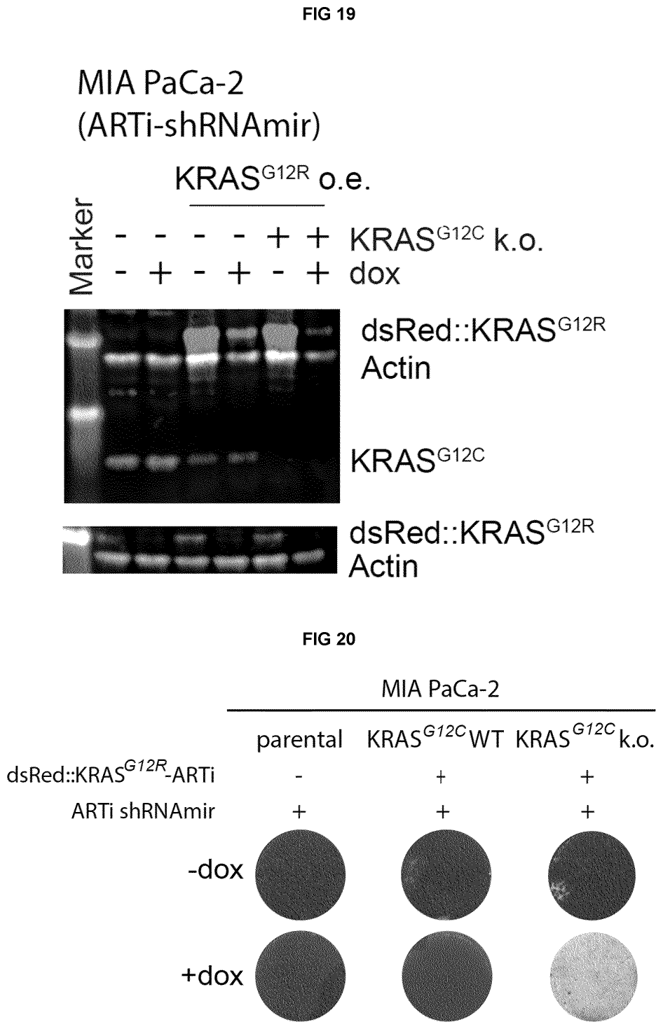

As the KRAS^{G12R} overexpression oncogene contains an ARTi target sequence downstream the STOP codon (dsRed::KRAS^{G12R}-ARTi), this construct is amenable to doxycycline induced knockdown. To validate the knockdown efficacy, we induced the expression of the ARTi-shRNAmir for 5 days and probed the dsRed::KRAS^{G12R} levels. An almost complete knockdown of dsRed::KRAS^{G12R} was observed, validating the excellent knockdown efficacy of the ARTi method (FIG. 19).

### Example 4.3: Validating KRASG12R Dependency In Vitro

Consistent with the excellent knockdown efficacy, loss of KRAS^{G12R} in the engineered cells was associated with impaired proliferation as determined by a proliferation assay and subsequent chrystal violet staining (FIG. 20; same assay procedures as described as in Example 3.4).

### Example 4.4: Validating KRASG12R Dependency In Vitro

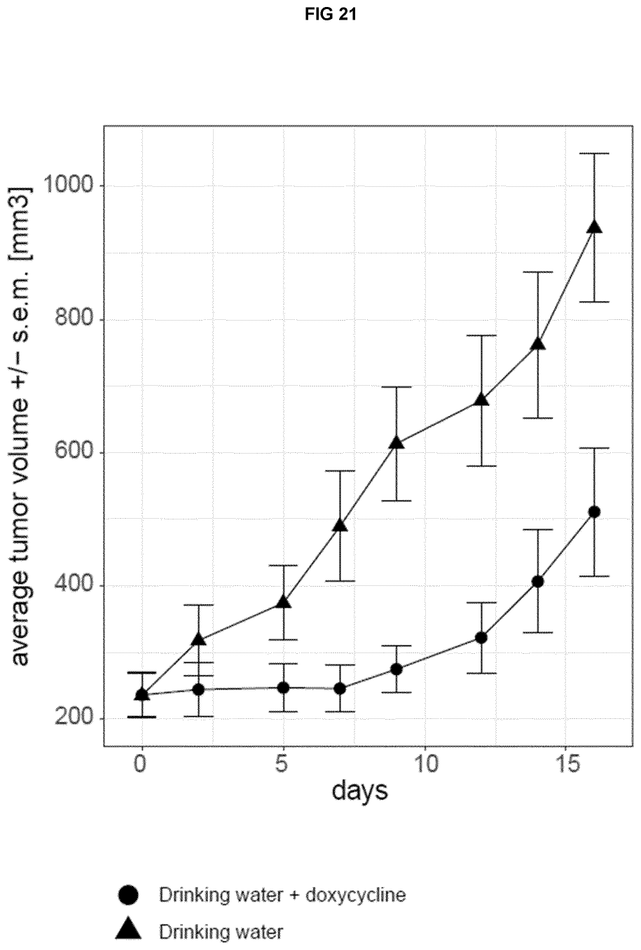

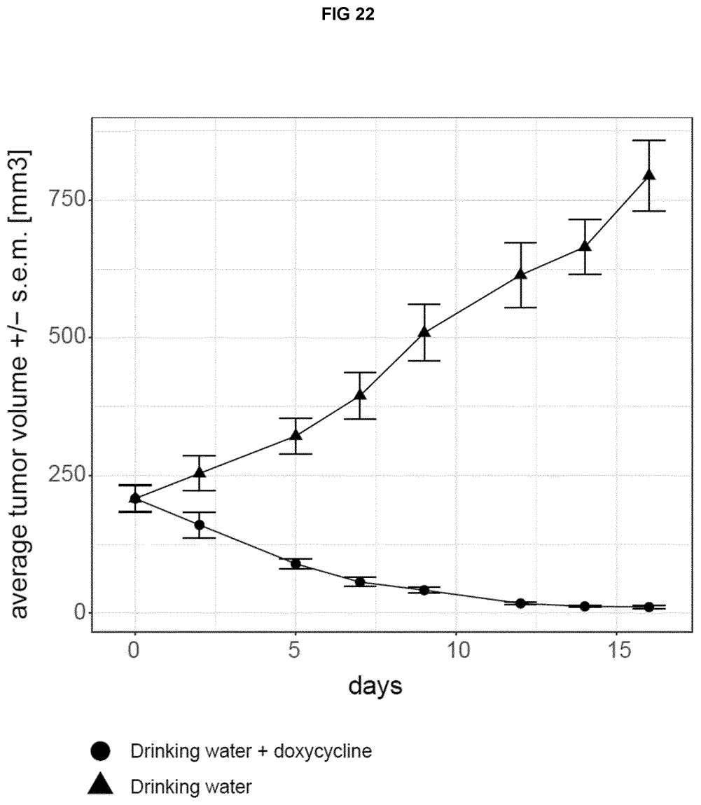

To determine if these cells could be used in an in vivo setting we used isogenic KRAS^{G12C} MIA PaCa-2 cells expressing the ARTi-shRNAmir and the dsRed::KRAS^{G12R}-ARTi construct. While the KRAS^{G12C} proficient cells grew under continuous doxycycline treatment (FIG. 21), the tumors formed by the KRAS^{G12C}deficient cells regressed to undetectable levels (FIG. 22). The delay in the onset of tumor growth in the KRAS^{G12C} proficient cells can potentially be explained by a simultaneous KRAS^{G12}C and KRAS^{G12R} dependency. Altogether, these results show that ARTi is an excellent method for setting up genetically validated, mechanistic tumor models.

### Example 5: Establishment of an In Vivo Benchmark Phenotype

### Example 5.1: Genome Engineering of STAG1 at Endogenous Locus to Generate a Homozygous STAG1::ARTi Allele

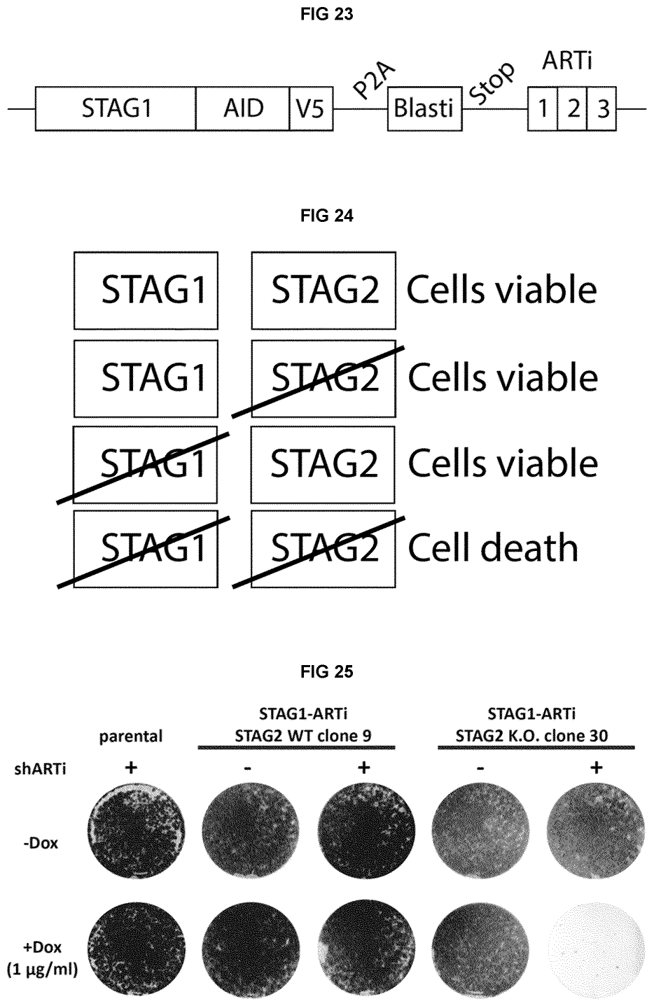

The paralog synthetic lethality of STAG1/2 was used to validate the specificity and potency of ARTi both in vivo and in vitro with the aim to generate an in vivo benchmark phenotype that candidate inhibitors of STAG1 will have to match. To utilize the ARTi approach for the establishment of an in vivo benchmark phenotype, isogenic STAG2 HCT 116 cells were engineered to contain the ARTi-shRNAmir target site (along with an AID and V5 tag and a Blasticidine resistance gene) homozygously inserted into the 3′-UTR of STAG1 (FIG. 23).

STAG2 mutations and deletions are observed in a subset of human cancers, rendering these cells dependent on the functionally redundant paralog STAG1 (FIG. 24). Untransformed cells or tumor cells with intact function of STAG2 are not dependent on STAG1, making STAG1 a prime candidate for targeted therapy.

### Example 5.2: Proliferation Assay and Subsequent Crystal Violet Staining of HCT 116 Cells in Absence or Presence of Doxycycline

ARTi mediated knockdown of STAG1 resulted in impaired growth of tumor cells deficient for STAG2 (FIG. 25). Cells were cultured in the presence or absence of doxycycline and were subsequently stained with crystal violet. ARTi mediated knockdown of STAG1 in the STAG2 deficient background lead to impairments in tumor cell proliferation, whereas STAG1 knockdown in the STAG2 proficient background did not result in a detectable phenotype in this assay.

### Example 5.3: Western Blot Confirmation of STAG1 Knock-In and Knockout of STAG2

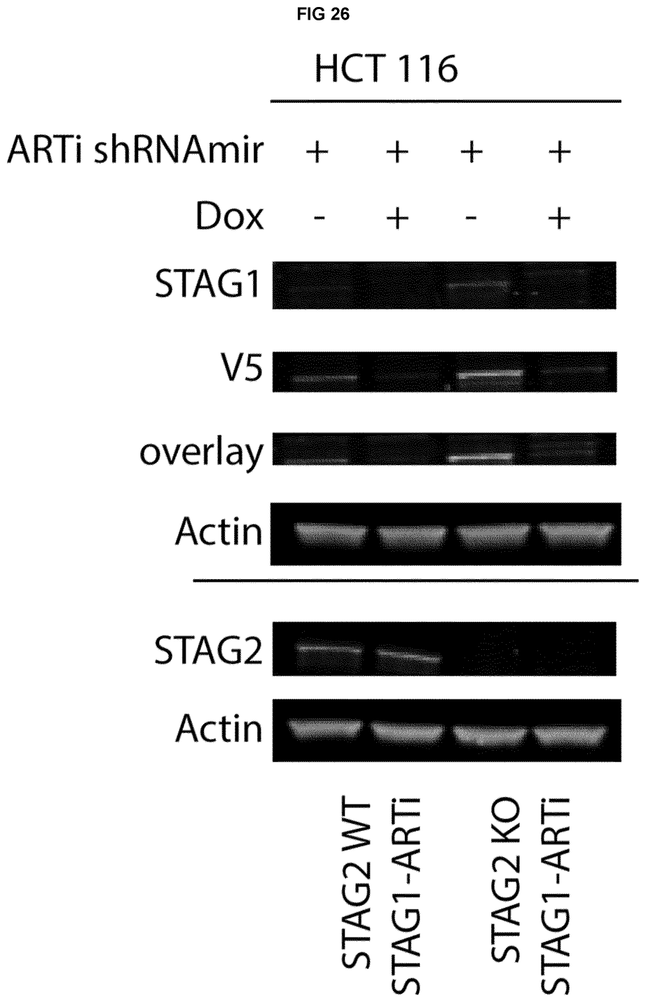

Western blotting confirmed both, the knockout of STAG2 as well as the insertion of the targeting cassette into the STAG 1 locus. Knockdown efficacy was validated by probing for endogenous STAG1 or V5, which was inserted into the STAG1 gene (FIG. 26). The cells were engineered into STAG2 proficient (STAG2WT) or deficient (STAG2^{k.o.}) cells. Both cell lines have the endogenous ARTi target site knock-in into the STAG1 locus and express the ARTi-shRNAmir. Induction of ARTi-shRNAmir expression with doxycycline is associated with the knockdown of STAG1.

### Example 5.4: In Vivo Experiment Comparing Growth Curves of Tumors Implanted with Engineered HCT 116 Cells in the Absence and Presence of Doxycycline

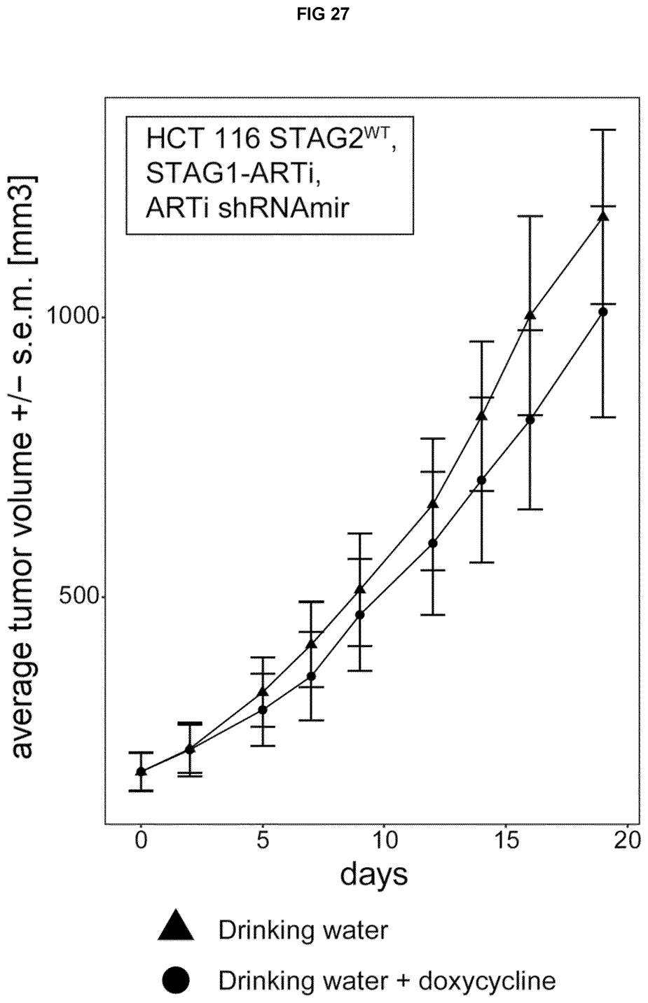

ARTi mediated knock-down in control tumors that express STAG2 resulted in tumor growth comparable to the non-doxycycline control group (FIG. 27), consistent with the concept that the remaining paralog STAG2 can compensate for the function.

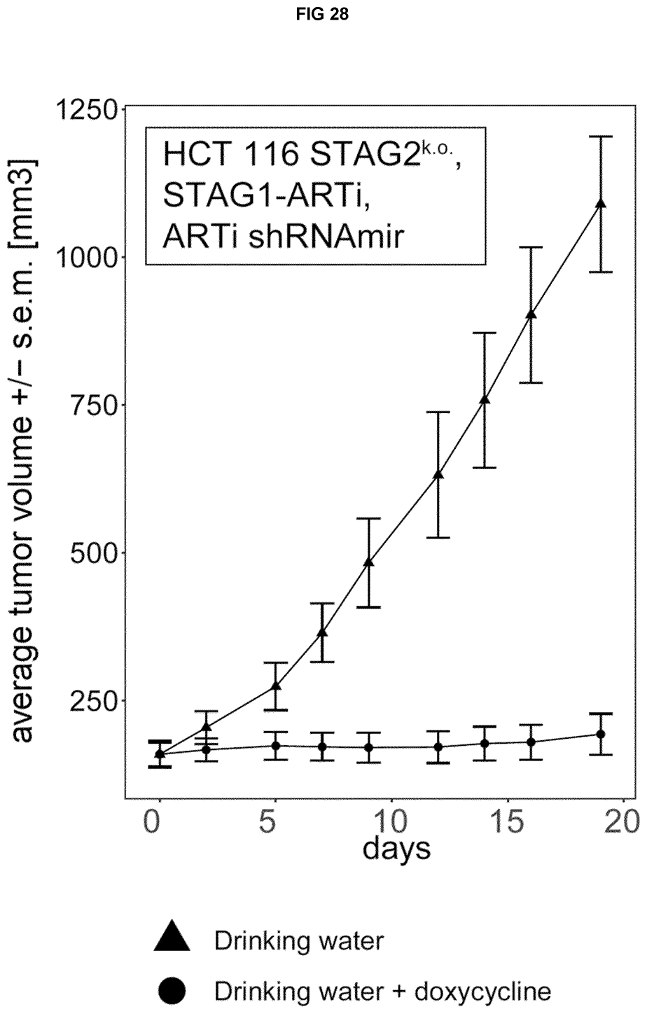

Doxycycline induced expression of an ARTi-shRNAmir resulted in an abrogation of tumor growth in mice harboring xeno-transplanted tumors in which STAG2 was knocked out using CRISPR/Cas9 and STAG1 was engineered to express ARTi-shRNAmir target sequences in the 3′-UTR of the endogenous STAG1 gene (FIG. 28). This phenotype is consistent with the in vitro and Western Blot data, demonstrating that STAG1 knockdown is associated with decreased tumor cell growth and proliferation depending on the STAG2 status.

### Sequence Listing Free Text

Table 1 below summarizes preferred nucleotide sequences of the invention, including inhibitory nucleic acids (DNA and RNA), artificial RNAi molecules, heterologous target sites and model target genes of the present invention.

TABLE 1
Nucleotide sequences and SEQ ID NOs of artificial RNAi molecules,
exogenous target sites and model target genes of the invention

| SEQ ID | Brief | |
|---|---|---|
| Number | description | Sequence |
| SEQ ID | ARTi generic | TTCGWWWNNAHHWWCATCCGGN |
| NO: 1 | antisense | W consists of A or T; |
| | (DNA) | H consists of A or T or C; |
| | | N consists of A or T or G or C |
| SEQ ID | ARTi generic | UUCGWWWNNAHHWWCAUCCGGN |
| NO: 2 | antisense | W consists of A or U; |
| | (RNA) | H consists of A or U or C; |
| | | N consists of A or U or G or C |
| SEQ ID | ARTi 6516 | TTCGAATTAAACTTCATCCGGA |
| NO: 3 | antisense | |
| | (DNA) | |
| SEQ ID | ARTi 6516 | UUCGAAUUAAACUUCAUCCGGA |
| NO: 4 | antisense | |
| | (RNA) | |
| SEQ ID | ARTi 6570 | TTCGATAACAATATCATCCGGA |
| NO: 5 | antisense | |
| | (DNA) | |
| SEQ ID | ARTi 6570 | UUCGAUAACAAUAUCAUCCGGA |
| NO: 6 | antisense | |
| | (RNA) | |
| SEQ ID | ARTi 6588 | TTCGATATAAACTTCATCCGGA |
| NO: 7 | antisense | |
| | (DNA) | |
| SEQ ID | ARTi 6588 | UUCGAUAUAAACUUCAUCCGGA |
| NO: 8 | antisense | |
| | (RNA) | |
| SEQ ID | ARTi 6634 | TTCGATTAAAACATCATCCGGA |
| NO: 9 | antisense | |
| | (DNA) | |
| SEQ ID | ARTi 6634 | UUCGAUUAAAACAUCAUCCGGA |
| NO: 10 | antisense | |
| | (RNA) | |
| SEQ ID | ARTi 6786 | TTCGTATACAATATCATCCGGA |
| NO: 11 | antisense | |
| | (DNA) | |
| SEQ ID | ARTi 6786 | UUCGUAUACAAUAUCAUCCGGA |
| NO: 12 | antisense | |
| | (RNA) | |
| SEQ ID | ARTi 6834 | TTCGTATGCAATATCATCCGGA |
| NO: 13 | antisense | |
| | (DNA) | |
| SEQ ID | ARTi 6834 | UUCGUAUGCAAUAUCAUCCGGA |
| NO: 14 | antisense | |
| | (RNA) | |
| SEQ ID | ARTi 6516 | TGCTGTTGACAGTGAGCGCCCGGATGAAGTTTAATTC |
| NO: 15 | 97 mer (DNA) | GAATAGTGAAGCCACAGATGTATTCGAATTAAACTTC |
| | | ATCCGGATGCCTACTGCCTCGGA |
| SEQ ID | ARTi 6516 | UGCUGUUGACAGUGAGCGCCCGGAUGAAGUUUAAU |
| NO: 16 | 97 mer (RNA) | UCGAAUAGUGAAGCCACAGAUGUAUUCGAAUUAAA |
| | | CUUCAUCCGGAUGCCUACUGCCUCGGA |
| SEQ ID | ARTi 6570 | TGCTGTTGACAGTGAGCGCCCGGATGATATTGTTATC |
| NO: 17 | 97 mer (DNA) | GAATAGTGAAGCCACAGATGTATTCGATAACAATATC |
| | | ATCCGGATGCCTACTGCCTCGGA |
| SEQ ID | ARTi 6570 | UGCUGUUGACAGUGAGCGCCCGGAUGAUAUUGUUA |
| NO: 18 | 97 mer (RNA) | UCGAAUAGUGAAGCCACAGAUGUAUUCGAUAACAA |
| | | UAUCAUCCGGAUGCCUACUGCCUCGGA |
| SEQ ID | ARTi 6588 | TGCTGTTGACAGTGAGCGCCCGGATGAAGTTTATATC |
| NO: 19 | 97 mer (DNA) | GAATAGTGAAGCCACAGATGTATTCGATATAAACTTC |
| | | ATCCGGATGCCTACTGCCTCGGA |
| SEQ ID | ARTi 6588 | UGCUGUUGACAGUGAGCGCCCGGAUGAAGUUUAUA |
| NO: 20 | 97 mer (RNA) | UCGAAUAGUGAAGCCACAGAUGUAUUCGAUAUAAA |
| | | CUUCAUCCGGAUGCCUACUGCCUCGGA |
| SEQ ID | ARTi 6634 | TGCTGTTGACAGTGAGCGCCCGGATGATGTTTTAATC |
| NO: 21 | 97 mer (DNA) | GAATAGTGAAGCCACAGATGTATTCGATTAAAACATC |
| | | ATCCGGATGCCTACTGCCTCGGA |
| SEQ ID | ARTi 6634 | UGCUGUUGACAGUGAGCGCCCGGAUGAUGUUUUAA |
| NO: 22 | 97 mer (RNA) | UCGAAUAGUGAAGCCACAGAUGUAUUCGAUUAAAA |
| | | CAUCAUCCGGAUGCCUACUGCCUCGGA |
| SEQ ID | ARTi 6786 | TGCTGTTGACAGTGAGCGCCCGGATGATATTGTATAC |
| NO: 23 | 97 mer (DNA) | GAATAGTGAAGCCACAGATGTATTCGTATACAATATC |
| | | ATCCGGATGCCTACTGCCTCGGA |
| SEQ ID | ARTi 6786 | UGCUGUUGACAGUGAGCGCCCGGAUGAUAUUGUAU |
| NO: 24 | 97 mer (RNA) | ACGAAUAGUGAAGCCACAGAUGUAUUCGUAUACAA |
| | | UAUCAUCCGGAUGCCUACUGCCUCGGA |
| SEQ ID | ARTi 6834 | TGCTGTTGACAGTGAGCGCCCGGATGATATTGCATA |
| NO: 25 | 97 mer (DNA) | CGAATAGTGAAGCCACAGATGTATTCGTATGCAATAT |
| | | CATCCGGATGCCTACTGCCTCGGA |
| SEQ ID | ARTi 6834 | UGCUGUUGACAGUGAGCGCCCGGAUGAUAUUGCAU |
| NO: 26 | 97 mer (RNA) | ACGAAUAGUGAAGCCACAGAUGUAUUCGUAUGCAA |
| | | UAUCAUCCGGAUGCCUACUGCCUCGGA |
| SEQ ID | ARTi generic | NCCGGATGWWDDTNNWWWCGAA |
| NO: 27 | target site | W consists of T or A; |
| | (DNA) | D consist of T or A or G; |
| | | N consist of T or A or C or G |
| SEQ ID | ARTi generic | NCCGGAUGWWDDUNNWWWCGAA |
| NO: 28 | target site | W consists of U or A; |
| | (RNA) | D consist of U or A or G; |
| | | N consist of U or A or C or G |
| SEQ ID | ARTi 6516 | TCCGGATGAAGTTTAATTCGAA |
| NO: 29 | target site | |
| | (DNA) | |
| SEQ ID | ARTi 6516 | UCCGGAUGAAGUUUAAUUCGAA |
| NO: 30 | target site | |
| | (RNA) | |
| SEQ ID | ARTi 6570 | TCCGGATGATATTGTTATCGAA |
| NO: 31 | target site | |
| | (DNA) | |
| SEQ ID | ARTi 6570 | UCCGGAUGAUAUUGUUAUCGAA |
| NO: 32 | target site | |
| | (RNA) | |
| SEQ ID | ARTi 6588 | TCCGGATGAAGTTTATATCGAA |
| NO: 33 | target site | |
| | (DNA) | |
| SEQ ID | ARTi 6588 | UCCGGAUGAAGUUUAUAUCGAA |
| NO: 34 | target site | |
| | (RNA) | |
| SEQ ID | ARTi 6634 | TCCGGATGATGTTTTAATCGAA |
| NO: 35 | target site | |
| | (DNA) | |
| SEQ ID | ARTi 6634 | UCCGGAUGAUGUUUUAAUCGAA |
| NO: 36 | target site | |
| | (RNA) | |
| SEQ ID | ARTi 6786 | TCCGGATGATATTGTATACGAA |
| NO: 37 | target site | |
| | (DNA) | |
| SEQ ID | ARTi 6786 | UCCGGAUGAUAUUGUAUACGAA |
| NO: 38 | target site | |
| | (RNA) | |
| SEQ ID | ARTi 6834 | TCCGGATGATATTGCATACGAA |
| NO: 39 | target site | |
| | (DNA) | |
| SEQ ID | ARTi 6834 | UCCGGAUGAUAUUGCAUACGAA |
| NO: 40 | target site | |
| | (RNA) | |
| SEQ ID | GPT | TAGGAATTATAATGCTTATCTACGTTTGACCGCCTGA |
| NO: 41 | (GFP::ARTi) | AGTCTCTGATTAAGTATCTGCCAGCTAAAGGTGAAGA |
| | 6516 target | TATATTCCCCATCCACTACAAGTACATGTGTAATAGCT |
| | site (DNA) | TTGCACAGGAATTTTGTTTAATATAACTCGAGCGANN |
| | | NNNNNNNNNNNNNNNNNNNN |
| | | wherein Ns are: TCCGGATGAAGTTTAATTCGAA |
| SEQ ID | GPT | UAGGAAUUAUAAUGCUUAUCUACGUUUGACCGCCU |
| NO: 42 | (GFP::ARTi) | GAAGUCUCUGAUUAAGUAUCUGCCAGCUAAAGGUG |
| | 6516 target | AAGAUAUAUUCCCCAUCCACUACAAGUACAUGUGUA |
| | site (RNA) | AUAGCUUUGCACAGGAAUUUUGUUUAAUAUAACUC |
| | | GAGCGANNNNNNNNNNNNNNNNNNNNNN |
| | | wherein Ns are: UCCGGAUGAAGUUUAAUUCGAA |
| SEQ ID | GPT | TAGGAATTATAATGCTTATCTACGTTTGACCGCCTGA |
| NO: 43 | (GFP::ARTi) | AGTCTCTGATTAAGTATCTGCCAGCTAAAGGTGAAGA |
| | 6570 target | TATATTCCCCATCCACTACAAGTACATGTGTAATAGCT |
| | site (DNA) | TTGCACAGGAATTTTGTTTAATATAACTCGAGCGANN |
| | | NNNNNNNNNNNNNNNNNNNN |
| | | wherein Ns are: TCCGGATGATATTGTTATCGAA |
| SEQ ID | GPT | UAGGAAUUAUAAUGCUUAUCUACGUUUGACCGCCU |
| NO: 44 | (GFP::ARTi) | GAAGUCUCUGAUUAAGUAUCUGCCAGCUAAAGGUG |
| | 6570 target | AAGAUAUAUUCCCCAUCCACUACAAGUACAUGUGUA |
| | site (RNA) | AUAGCUUUGCACAGGAAUUUUGUUUAAUAUAACUC |
| | | GAGCGANNNNNNNNNNNNNNNNNNNNNN |
| | | wherein Ns are: UCCGGAUGAUAUUGUUAUCGAA |
| SEQ ID | GPT | TAGGAATTATAATGCTTATCTACGTTTGACCGCCTGA |
| NO: 45 | (GFP::ARTi) | AGTCTCTGATTAAGTATCTGCCAGCTAAAGGTGAAGA |
| | 6588 target | TATATTCCCCATCCACTACAAGTACATGTGTAATAGCT |
| | site (DNA) | TTGCACAGGAATTTTGTTTAATATAACTCGAGCGANN |
| | | NNNNNNNNNNNNNNNNNNNN |
| | | wherein Ns are: TCCGGATGAAGTTTATATCGAA |
| SEQ ID | GPT | UAGGAAUUAUAAUGCUUAUCUACGUUUGACCGCCU |
| NO: 46 | (GFP::ARTi) | GAAGUCUCUGAUUAAGUAUCUGCCAGCUAAAGGUG |
| | 6588 target | AAGAUAUAUUCCCCAUCCACUACAAGUACAUGUGUA |
| | site (RNA) | AUAGCUUUGCACAGGAAUUUUGUUUAAUAUAACUC |
| | | GAGCGANNNNNNNNNNNNNNNNNNNNNN |
| | | wherein Ns are: UCCGGAUGAAGUUUAUAUCGAA |
| SEQ ID | GPT | TAGGAATTATAATGCTTATCTACGTTTGACCGCCTGA |
| NO: 47 | (GFP::ARTi) | AGTCTCTGATTAAGTATCTGCCAGCTAAAGGTGAAGA |
| | | TATATTCCCCATCCACTACAAGTACATGTGTAATAGCT |
| | 6634 target | TTGCACAGGAATTTTGTTTAATATAACTCGAGCGANN |
| | | NNNNNNNNNNNNNNNNNNNN |
| | site (DNA) | wherein Ns are: TCCGGATGATGTTTTAATCGAA |
| SEQ ID | GPT | UAGGAAUUAUAAUGCUUAUCUACGUUUGACCGCCU |
| NO: 48 | (GFP::ARTi) | GAAGUCUCUGAUUAAGUAUCUGCCAGCUAAAGGUG |
| | 6634 target | AAGAUAUAUUCCCCAUCCACUACAAGUACAUGUGUA |
| | site (RNA) | AUAGCUUUGCACAGGAAUUUUGUUUAAUAUAACUC |
| | | GAGCGANNNNNNNNNNNNNNNNNNNNNN |
| | | wherein Ns are: UCCGGAUGAUGUUUUAAUCGAA |
| SEQ ID | GPT | TAGGAATTATAATGCTTATCTACGTTTGACCGCCTGA |
| NO: 49 | (GFP::ARTi) | AGTCTCTGATTAAGTATCTGCCAGCTAAAGGTGAAGA |
| | 6786 target | TATATTCCCCATCCACTACAAGTACATGTGTAATAGCT |
| | site (DNA) | TTGCACAGGAATTTTGTTTAATATAACTCGAGCGANN |
| | | NNNNNNNNNNNNNNNNNNNN |
| | | wherein Ns are: TCCGGATGATATTGTATACGAA |
| SEQ ID | GPT | UAGGAAUUAUAAUGCUUAUCUACGUUUGACCGCCU |
| NO: 50 | (GFP::ARTi) | GAAGUCUCUGAUUAAGUAUCUGCCAGCUAAAGGUG |
| | 6786 target | AAGAUAUAUUCCCCAUCCACUACAAGUACAUGUGUA |
| | site (RNA) | AUAGCUUUGCACAGGAAUUUUGUUUAAUAUAACUC |
| | | GAGCGANNNNNNNNNNNNNNNNNNNNNN |
| | | wherein Ns are: UCCGGAUGAUAUUGUAUACGAA |
| SEQ ID | GPT | TAGGAATTATAATGCTTATCTACGTTTGACCGCCTGA |
| NO: 51 | (GFP::ARTi) | AGTCTCTGATTAAGTATCTGCCAGCTAAAGGTGAAGA |
| | 6834 target | TATATTCCCCATCCACTACAAGTACATGTGTAATAGCT |
| | site (DNA) | TTGCACAGGAATTTTGTTTAATATAACTCGAGCGANN |
| | | NNNNNNNNNNNNNNNNNNNN |
| | | wherein Ns are: TCCGGATGATATTGCATACGAA |
| SEQ ID | GPT | UAGGAAUUAUAAUGCUUAUCUACGUUUGACCGCCU |
| NO: 52 | (GFP::ARTi) | GAAGUCUCUGAUUAAGUAUCUGCCAGCUAAAGGUG |
| | 6834 target | AAGAUAUAUUCCCCAUCCACUACAAGUACAUGUGUA |
| | site (RNA) | AUAGCUUUGCACAGGAAUUUUGUUUAAUAUAACUC |
| | | GAGCGANNNNNNNNNNNNNNNNNNNNNN |
| | | wherein Ns are: UCCGGAUGAUAUUGCAUACGAA |
| SEQ ID | EGFRdel19:: | ATGCGACCAAGCGGAACCGCTGGAGCCGCTCTGCT |
| NO: 53 | Linker::V5:: | GGCTCTGCTGGCCGCCCTGTGCCCTGCCAGCAGGG |
| | dsRed::ARTi | CTCTGGAGGAAAAGAAAGTGTGCCAGGGGACATCCA |
| | target sites- | ACAAGCTGACACAGCTGGGAACTTTCGAGGATCACT |
| | [STOP] | TTCTGAGCCTGCAGAGAATGTTTAACAATTGCGAAGT |
| | (DNA) | GGTGCTGGGCAACCTGGAGATCACTTACGTGCAGAG |
| | | GAATTATGACCTGAGCTTCCTGAAGACCATTCAGGAA |
| | | GTGGCCGGATACGTGCTGATCGCTCTGAACACAGTG |
| | | GAACGGATCCCCCTGGAGAATCTGCAGATCATTAGG |
| | | GGCAACATGTACTATGAGAACAGCTACGCCCTGGCT |
| | | GTGCTGAGCAACTATGACGCCAATAAGACCGGGCTG |
| | | AAAGAACTGCCAATGAGAAATCTGCAGGAGATCCTG |
| | | CATGGAGCCGTGAGGTTCTCAAACAATCCCGCCCTG |
| | | TGCAACGTGGAGAGCATCCAGTGGAGGGACATTGTG |
| | | AGCTCCGATTTCCTGAGCAACATGTCCATGGATTTTC |
| | | AGAATCACCTGGGGTCCTGCCAGAAGTGTGACCCAA |
| | | GTTGTCCCAACGGATCATGCTGGGGAGCCGGCGAG |
| | | GAAAATTGCCAGAAGCTGACTAAAATCATTTGCGCTC |
| | | AGCAGTGTTCCGGCCGCTGCAGGGGGAAATCTCCTA |
| | | GTGATTGCTGTCATAATCAGTGTGCTGCTGGATGCAC |
| | | CGGACCACGCGAATCTGACTGCCTGGTGTGCCGCAA |
| | | GTTTCGGGACGAGGCCACCTGTAAAGATACATGCCC |
| | | CCCTCTGATGCTGTACAACCCCACCACATATCAGATG |
| | | GATGTGAATCCTGAGGGGAAGTACTCCTTCGGAGCC |
| | | ACCTGCGTGAAGAAGTGCCCTCGCAACTATGTGGTG |
| | | ACAGACCACGGAAGCTGCGTGCGGGCTTGCGGAGC |
| | | TGACTCCTACGAAATGGAGGAAGATGGCGTGCGCAA |
| | | GTGCAAGAAATGTGAGGGGCCATGTCGGAAAGTGTG |
| | | CAATGGGATCGGAATTGGCGAGTTCAAGGATTCACT |
| | | GAGCATCAACGCCACCAATATTAAGCACTTCAAGAAC |
| | | TGCACATCCATCTCTGGAGATCTGCACATTCTGCCCG |
| | | TGGCCTTCAGAGGCGACTCTTTTACTCACACCCCACC |
| | | CCTGGACCCTCAGGAACTGGATATCCTGAAGACTGT |
| | | GAAAGAGATCACCGGCTTCCTGCTGATTCAGGCCTG |
| | | GCCTGAGAACAGGACAGACCTGCATGCTTTTGAGAA |
| | | TCTGGAAATCATTAGAGGGAGGACTAAGCAGCACGG |
| | | ACAGTTCAGCCTGGCCGTGGTGTCCCTGAACATCAC |
| | | TTCTCTGGGCCTGCGCAGTCTGAAGGAGATTTCAGA |
| | | CGGCGATGTGATCATTAGCGGGAACAAAAATCTGTG |
| | | CTACGCCAACACCATCAATTGGAAGAAACTGTTTGGG |
| | | ACATCCGGACAGAAGACTAAAATCATTTCTAACAGGG |
| | | GAGAGAATAGTTGTAAGGCCACAGGCCAGGTGTGCC |
| | | ATGCTCTGTGCTCCCCAGAAGGATGCTGGGGACCAG |
| | | AGCCTCGCGATTGCGTGAGCTGTAGGAACGTGTCCC |
| | | GCGGCAGGGAATGCGTGGACAAGTGCAATCTGCTG |
| | | GAGGGGGAACCAAGAGAGTTCGTGGAAAACTCTGAG |
| | | TGCATCCAGTGTCACCCTGAGTGTCTGCCACAGGCC |
| | | ATGAACATTACATGCACTGGGAGGGGACCCGACAAT |
| | | TGCATCCAGTGTGCCCATTACATTGATGGCCCCCACT |
| | | GCGTGAAGACATGCCCTGCTGGAGTGATGGGCGAG |
| | | AACAATACTCTGGTGTGGAAATACGCCGACGCTGGA |
| | | CACGTGTGCCACCTGTGCCACCCCAATTGTACTTATG |
| | | GATGCACCGGACCAGGACTGGAGGGATGCCCTACTA |
| | | ACGGGCCTAAGATCCCAAGCATTGCCACCGGGATGG |
| | | TGGGAGCTCTGCTGCTGCTGCTGGTGGTGGCCCTG |
| | | GGAATCGGCCTGTTTATGCGGAGAAGGCACATTGTG |
| | | AGAAAAAGGACCCTGCGCCGGCTGCTGCAGGAGAG |
| | | GGAACTGGTGGAACCCCTGACACCTTCTGGAGAGGC |
| | | CCCTAACCAGGCTCTGCTGCGGATCCTGAAGGAGAC |
| | | TGAGTTCAAGAAGATCAAGGTGCTGGGGAGTGGAGC |
| | | CTTTGGAACCGTGTACAAGGGCCTGTGGATCCCAGA |
| | | GGGGGAAAAGGTGAAAATCCCCGTGGCCATCAAGAC |
| | | ATCTCCTAAGGCTAACAAGGAAATCCTGGACGAGGC |
| | | CTATGTGATGGCTAGTGTGGATAACCCACACGTGTG |
| | | CCGCCTGCTGGGCATCTGCCTGACCTCAACAGTGCA |
| | | GCTGATTACCCAGCTGATGCCCTTCGGGTGTCTGCT |
| | | GGACTACGTGCGGGAACACAAGGATAACATCGGAAG |
| | | CCAGTATCTGCTGAATTGGTGCGTGCAGATTGCCAAA |
| | | GGCATGAATTACCTGGAGGACAGAAGGCTGGTGCAT |
| | | AGAGATCTGGCCGCTAGGAACGTGCTGGTGAAGACA |
| | | CCCCAGCACGTGAAAATCACTGATTTTGGACTGGCC |
| | | AAGCTGCTGGGCGCTGAGGAAAAAGAATATCATGCC |
| | | GAGGGCGGGAAGGTGCCCATCAAGTGGATGGCTCT |
| | | GGAGAGCATCCTGCATCGGATCTACACACACCAGAG |
| | | CGACGTGTGGTCCTATGGCGTGACCGTGTGGGAACT |
| | | GATGACCTTCGGGTCTAAGCCCTACGACGGAATCCC |
| | | TGCCAGTGAGATCTCTAGTATTCTGGAAAAAGGGGA |
| | | GAGACTGCCACAGCCTCCAATCTGTACCATTGACGT |
| | | GTATATGATCATGGTGAAGTGCTGGATGATTGACGCC |
| | | GATAGTAGACCAAAGTTCAGGGAACTGATCATTGAGT |
| | | TTTCAAAAATGGCTCGCGACCCCCAGCGGTACCTGG |
| | | TCATCCAGGGCGATGAAAGAATGCACCTGCCATCTC |
| | | CCACCGACAGTAACTTCTACAGGGCCCTGATGGACG |
| | | AGGAAGATATGGACGATGTGGTGGACGCTGATGAGT |
| | | ATCTGATCCCCCAGCAGGGCTTCTTTTCAAGCCCAAG |
| | | TACTTCACGGACCCCCCTGCTGTCCTCTCTGTCTGCC |
| | | ACCAGTAACAATTCAACAGTGGCTTGTATCGATCGCA |
| | | ACGGCCTGCAGTCCTGCCCAATTAAGGAGGACTCTT |
| | | TTCTGCAGCGGTATAGTTCAGACCCCACAGGGGCCC |
| | | TGACTGAAGATTCCATCGACGATACCTTCCTGCCTGT |
| | | GCCAGAGTACATTAATCAGAGTGTGCCAAAGAGGCC |
| | | AGCTGGATCAGTGCAGAACCCCGTGTACCACAACCA |
| | | GCCACTGAATCCAGCTCCTTCCCGGGACCCACATTA |
| | | CCAGGATCCCCACTCTACAGCTGTGGGGAACCCTGA |
| | | GTATCTGAATACCGTGCAGCCAACATGCGTGAACAG |
| | | CACCTTCGACAGCCCTGCCCATTGGGCTCAGAAGGG |
| | | ATCCCACCAGATCTCTCTGGACAACCCAGATTACCAG |
| | | CAGGACTTCTTTCCTAAGGAAGCCAAACCAAACGGAA |
| | | TCTTCAAGGGCAGCACCGCCGAAAATGCTGAGTATC |
| | | TGAGAGTGGCCCCTCAGAGCTCCGAGTTCATCGGCG |
| | | CTGGCGGCGGCGGCAGCGGCGGCGGCGGCTCCGG |
| | | CGGCGGCGGCTCTGGCGGCAAACCTATTCCTAACCC |
| | | TCTGCTGGGACTGGACTCAACTATGGCAAGTAGCGA |
| | | GAATGTGATTACAGAGTTTATGAGGTTCAAAGTCAGG |
| | | ATGGAGGGGACCGTCAATGGGCACGAGTTCGAGATT |
| | | GAGGGAGAGGGAGAGGGCAGGCCTTATGAGGGCCA |
| | | CAACACCGTGAAGCTGAAGGTGACAAAGGGAGGACC |
| | | ACTGCCTTTCGCCTGGGACATCCTGTCTCCACAGTTT |
| | | CAGTATGGCAGCAAGGTGTACGTGAAGCACCCAGCC |
| | | GACATCCCCGATTACAAGAAGCTGTCTTTCCCCGAG |
| | | GGCTTTAAGTGGGAGAGAGTGATGAACTTCGAGGAC |
| | | GGAGGAGTGGCAACCGTGACACAGGACAGCTCCCT |
| | | GCAGGATGGCTGCTTTATCTATAAGGTGAAGTTCATC |
| | | GGCGTGAATTTTCCTTCCGATGGCCCAGTGATGCAG |
| | | AAGAAGACCATGGGCTGGGAGGCCTCTACAGAGCG |
| | | GCTGTACCCCAGAGACGGCGTGCTGAAGGGCGAGA |
| | | CACACAAGGCCCTGAAGCTGAAGGATGGCGGCCACT |
| | | ATCTGGTGGAGTTCAAGAGCATCTACATGGCCAAGA |
| | | AGCCCGTGCAGCTGCCTGGCTACTATTACGTGGACG |
| | | CCAAGCTGGATATCACATCCCACAATGAAGATTACAC |
| | | CATCGTGGAGCAGTATGAACGCACCGAAGGGAGGCA |
| | | TCACCTGTTTCTGATCCGGATGATATTGTATACGAAT |
| | | CCGGATGATATTGTTATCGAA[STOP] |
| SEQ ID | EGFRdel19:: | AUGCGACCAAGCGGAACCGCUGGAGCCGCUCUGCU |
| NO: 54 | Linker::V5:: | GGCUCUGCUGGCCGCCCUGUGCCCUGCCAGCAGG |
| | dsRed::ARTi | GCUCUGGAGGAAAAGAAAGUGUGCCAGGGGACAUC |
| | target sites- | CAACAAGCUGACACAGCUGGGAACUUUCGAGGAUC |
| | [STOP] | ACUUUCUGAGCCUGCAGAGAAUGUUUAACAAUUGC |
| | (RNA) | GAAGUGGUGCUGGGCAACCUGGAGAUCACUUACGU |
| | | GCAGAGGAAUUAUGACCUGAGCUUCCUGAAGACCA |
| | | UUCAGGAAGUGGCCGGAUACGUGCUGAUCGCUCUG |
| | | AACACAGUGGAACGGAUCCCCCUGGAGAAUCUGCA |
| | | GAUCAUUAGGGGCAACAUGUACUAUGAGAACAGCU |
| | | ACGCCCUGGCUGUGCUGAGCAACUAUGACGCCAAU |
| | | AAGACCGGGCUGAAAGAACUGCCAAUGAGAAAUCU |
| | | GCAGGAGAUCCUGCAUGGAGCCGUGAGGUUCUCAA |
| | | ACAAUCCCGCCCUGUGCAACGUGGAGAGCAUCCAG |
| | | UGGAGGGACAUUGUGAGCUCCGAUUUCCUGAGCAA |
| | | CAUGUCCAUGGAUUUUCAGAAUCACCUGGGGUCCU |
| | | GCCAGAAGUGUGACCCAAGUUGUCCCAACGGAUCA |
| | | UGCUGGGGAGCCGGCGAGGAAAAUUGCCAGAAGCU |
| | | GACUAAAAUCAUUUGCGCUCAGCAGUGUUCCGGCC |
| | | GCUGCAGGGGGAAAUCUCCUAGUGAUUGCUGUCAU |
| | | AAUCAGUGUGCUGCUGGAUGCACCGGACCACGCGA |
| | | AUCUGACUGCCUGGUGUGCCGCAAGUUUCGGGACG |
| | | AGGCCACCUGUAAAGAUACAUGCCCCCCUCUGAUG |
| | | CUGUACAACCCCACCACAUAUCAGAUGGAUGUGAAU |
| | | CCUGAGGGGAAGUACUCCUUCGGAGCCACCUGCGU |
| | | GAAGAAGUGCCCUCGCAACUAUGUGGUGACAGACC |
| | | ACGGAAGCUGCGUGCGGGCUUGCGGAGCUGACUC |
| | | CUACGAAAUGGAGGAAGAUGGCGUGCGCAAGUGCA |
| | | AGAAAUGUGAGGGGCCAUGUCGGAAAGUGUGCAAU |
| | | GGGAUCGGAAUUGGCGAGUUCAAGGAUUCACUGAG |
| | | CAUCAACGCCACCAAUAUUAAGCACUUCAAGAACUG |
| | | CACAUCCAUCUCUGGAGAUCUGCACAUUCUGCCCG |
| | | UGGCCUUCAGAGGCGACUCUUUUACUCACACCCCA |
| | | CCCCUGGACCCUCAGGAACUGGAUAUCCUGAAGAC |
| | | UGUGAAAGAGAUCACCGGCUUCCUGCUGAUUCAGG |
| | | CCUGGCCUGAGAACAGGACAGACCUGCAUGCUUUU |
| | | GAGAAUCUGGAAAUCAUUAGAGGGAGGACUAAGCA |
| | | GCACGGACAGUUCAGCCUGGCCGUGGUGUCCCUGA |
| | | ACAUCACUUCUCUGGGCCUGCGCAGUCUGAAGGAG |
| | | AUUUCAGACGGCGAUGUGAUCAUUAGCGGGAACAA |
| | | AAAUCUGUGCUACGCCAACACCAUCAAUUGGAAGAA |
| | | ACUGUUUGGGACAUCCGGACAGAAGACUAAAAUCA |
| | | UUUCUAACAGGGGAGAGAAUAGUUGUAAGGCCACA |
| | | GGCCAGGUGUGCCAUGCUCUGUGCUCCCCAGAAGG |
| | | AUGCUGGGGACCAGAGCCUCGCGAUUGCGUGAGCU |
| | | GUAGGAACGUGUCCCGCGGCAGGGAAUGCGUGGA |
| | | CAAGUGCAAUCUGCUGGAGGGGGAACCAAGAGAGU |
| | | UCGUGGAAAACUCUGAGUGCAUCCAGUGUCACCCU |
| | | GAGUGUCUGCCACAGGCCAUGAACAUUACAUGCAC |
| | | UGGGAGGGGACCCGACAAUUGCAUCCAGUGUGCCC |
| | | AUUACAUUGAUGGCCCCCACUGCGUGAAGACAUGC |
| | | CCUGCUGGAGUGAUGGGCGAGAACAAUACUCUGGU |
| | | GUGGAAAUACGCCGACGCUGGACACGUGUGCCACC |
| | | UGUGCCACCCCAAUUGUACUUAUGGAUGCACCGGA |
| | | CCAGGACUGGAGGGAUGCCCUACUAACGGGCCUAA |
| | | GAUCCCAAGCAUUGCCACCGGGAUGGUGGGAGCUC |
| | | UGCUGCUGCUGCUGGUGGUGGCCCUGGGAAUCGG |
| | | CCUGUUUAUGCGGAGAAGGCACAUUGUGAGAAAAA |
| | | GGACCCUGCGCCGGCUGCUGCAGGAGAGGGAACU |
| | | GGUGGAACCCCUGACACCUUCUGGAGAGGCCCCUA |
| | | ACCAGGCUCUGCUGCGGAUCCUGAAGGAGACUGAG |
| | | UUCAAGAAGAUCAAGGUGCUGGGGAGUGGAGCCUU |
| | | UGGAACCGUGUACAAGGGCCUGUGGAUCCCAGAGG |
| | | GGGAAAAGGUGAAAAUCCCCGUGGCCAUCAAGACA |
| | | UCUCCUAAGGCUAACAAGGAAAUCCUGGACGAGGC |
| | | CUAUGUGAUGGCUAGUGUGGAUAACCCACACGUGU |
| | | GCCGCCUGCUGGGCAUCUGCCUGACCUCAACAGUG |
| | | CAGCUGAUUACCCAGCUGAUGCCCUUCGGGUGUCU |
| | | GCUGGACUACGUGCGGGAACACAAGGAUAACAUCG |
| | | GAAGCCAGUAUCUGCUGAAUUGGUGCGUGCAGAUU |
| | | GCCAAAGGCAUGAAUUACCUGGAGGACAGAAGGCU |
| | | GGUGCAUAGAGAUCUGGCCGCUAGGAACGUGCUGG |
| | | UGAAGACACCCCAGCACGUGAAAAUCACUGAUUUU |
| | | GGACUGGCCAAGCUGCUGGGCGCUGAGGAAAAAGA |
| | | AUAUCAUGCCGAGGGCGGGAAGGUGCCCAUCAAGU |
| | | GGAUGGCUCUGGAGAGCAUCCUGCAUCGGAUCUAC |
| | | ACACACCAGAGCGACGUGUGGUCCUAUGGCGUGAC |
| | | CGUGUGGGAACUGAUGACCUUCGGGUCUAAGCCCU |
| | | ACGACGGAAUCCCUGCCAGUGAGAUCUCUAGUAUU |
| | | CUGGAAAAAGGGGAGAGACUGCCACAGCCUCCAAU |
| | | CUGUACCAUUGACGUGUAUAUGAUCAUGGUGAAGU |
| | | GCUGGAUGAUUGACGCCGAUAGUAGACCAAAGUUC |
| | | AGGGAACUGAUCAUUGAGUUUUCAAAAAUGGCUCG |
| | | CGACCCCCAGCGGUACCUGGUCAUCCAGGGCGAUG |
| | | AAAGAAUGCACCUGCCAUCUCCCACCGACAGUAACU |
| | | UCUACAGGGCCCUGAUGGACGAGGAAGAUAUGGAC |
| | | GAUGUGGUGGACGCUGAUGAGUAUCUGAUCCCCCA |
| | | GCAGGGCUUCUUUUCAAGCCCAAGUACUUCACGGA |
| | | CCCCCCUGCUGUCCUCUCUGUCUGCCACCAGUAAC |
| | | AAUUCAACAGUGGCUUGUAUCGAUCGCAACGGCCU |
| | | GCAGUCCUGCCCAAUUAAGGAGGACUCUUUUCUGC |
| | | AGCGGUAUAGUUCAGACCCCACAGGGGCCCUGACU |
| | | GAAGAUUCCAUCGACGAUACCUUCCUGCCUGUGCC |
| | | AGAGUACAUUAAUCAGAGUGUGCCAAAGAGGCCAG |
| | | CUGGAUCAGUGCAGAACCCCGUGUACCACAACCAG |
| | | CCACUGAAUCCAGCUCCUUCCCGGGACCCACAUUA |
| | | CCAGGAUCCCCACUCUACAGCUGUGGGGAACCCUG |
| | | AGUAUCUGAAUACCGUGCAGCCAACAUGCGUGAAC |
| | | AGCACCUUCGACAGCCCUGCCCAUUGGGCUCAGAA |
| | | GGGAUCCCACCAGAUCUCUCUGGACAACCCAGAUU |
| | | ACCAGCAGGACUUCUUUCCUAAGGAAGCCAAACCAA |
| | | ACGGAAUCUUCAAGGGCAGCACCGCCGAAAAUGCU |
| | | GAGUAUCUGAGAGUGGCCCCUCAGAGCUCCGAGUU |
| | | CAUCGGCGCUGGCGGCGGCGGCAGCGGCGGCGGC |
| | | GGCUCCGGCGGCGGCGGCUCUGGCGGCAAACCUA |
| | | UUCCUAACCCUCUGCUGGGACUGGACUCAACUAUG |
| | | GCAAGUAGCGAGAAUGUGAUUACAGAGUUUAUGAG |
| | | GUUCAAAGUCAGGAUGGAGGGGACCGUCAAUGGGC |
| | | ACGAGUUCGAGAUUGAGGGAGAGGGAGAGGGCAG |
| | | GCCUUAUGAGGGCCACAACACCGUGAAGCUGAAGG |
| | | UGACAAAGGGAGGACCACUGCCUUUCGCCUGGGAC |
| | | AUCCUGUCUCCACAGUUUCAGUAUGGCAGCAAGGU |
| | | GUACGUGAAGCACCCAGCCGACAUCCCCGAUUACA |
| | | AGAAGCUGUCUUUCCCCGAGGGCUUUAAGUGGGAG |
| | | AGAGUGAUGAACUUCGAGGACGGAGGAGUGGCAAC |
| | | CGUGACACAGGACAGCUCCCUGCAGGAUGGCUGCU |
| | | UUAUCUAUAAGGUGAAGUUCAUCGGCGUGAAUUUU |
| | | CCUUCCGAUGGCCCAGUGAUGCAGAAGAAGACCAU |
| | | GGGCUGGGAGGCCUCUACAGAGCGGCUGUACCCCA |
| | | GAGACGGCGUGCUGAAGGGCGAGACACACAAGGCC |
| | | CUGAAGCUGAAGGAUGGCGGCCACUAUCUGGUGGA |
| | | GUUCAAGAGCAUCUACAUGGCCAAGAAGCCCGUGC |
| | | AGCUGCCUGGCUACUAUUACGUGGACGCCAAGCUG |
| | | GAUAUCACAUCCCACAAUGAAGAUUACACCAUCGUG |
| | | GAGCAGUAUGAACGCACCGAAGGGAGGCAUCACCU |
| | | GUUUCUGAUCCGGAUGAUAUUGUAUACGAAUCCGG |
| | | AUGAUAUUGUUAUCGAA[STOP] |
| SEQ ID | STAG1 | GGAGGTGGATCGGGAGGTGGATCGCCTAAAGATCCA |
| NO: 57 | (endogenous | GCCAAACCTCCGGCCAAGGCACAAGTIGTGGGATGG |
| | sequence not | CCACCGGTGAGATCATACCGGAAGAACGTGATGGTT |
| | displayed):: | TCCTGCCAAAAATCAAGCGGTGGCCCGGAGGCGGC |
| | Linker::AID:: | GGCGTTCGTGAAGAGCGCTGGGAAGCCCATCCCCAA |
| | V5::P2A::Blasti- | CCCCCTGCTGGGCCTGGACAGCACGGGCAGCGGCG |
| | [STOP]-ARTi | CCACCAACTTCAGCCTGCTGAAGCAGGCCGGCGACG |
| | target sites | TGGAGGAGAACCCCGGCCCCATGAAGACCTTCAACA |
| | (DNA) | TCTCTCAGCAGGACCTTGAGCTGGTGGAAGTTGCCA |
| | | CTGAGAAAATCACCATGCTCTATGAGGACAACAAGCA |
| | | CCATGTTGGTGCTGCCATCAGGACCAAGACAGGAGA |
| | | AATCATCTCTGCTGTCCACATTGAAGCCTACATTGGC |
| | | AGGGTCACTGTGTGTGCAGAGGCCATTGCCATTGGG |
| | | TCTGCAGTCTCCAATGGGCAGAAGGACTTTGACACC |
| | | ATTGTGGCTGTGAGGCACCCCTACAGTGATGAGGTG |
| | | GACAGGTCCATCAGAGTGGTGTCCCCCTGTGGCATG |
| | | TGCAGGGAGCTCATCTCAGACTATGCCCCTGATTGC |
| | | TTTGTTCTGATTGAGATGAATGGCAAGCTGGTCAAGA |
| | | CAACCATTGAGGAGCTGATTCCACTGAAATACACCAG |
| | | AAACTGA[STOP]TCCGGATGATATTGTATACGAATCC |
| | | GGATGATGTTTTAATCGAATCCGGATGATATTGTTAT |
| | | CGAA |
| SEQ ID | STAG1 | GGAGGUGGAUCGGGAGGUGGAUCGCCUAAAGAUCC |
| NO: 58 | (endogenous | AGCCAAACCUCCGGCCAAGGCACAAGUUGUGGGAU |
| | sequence not | GGCCACCGGUGAGAUCAUACCGGAAGAACGUGAUG |
| | displayed):: | GUUUCCUGCCAAAAAUCAAGCGGUGGCCCGGAGGC |
| | Linker::AID:: | GGCGGCGUUCGUGAAGAGCGCUGGGAAGCCCAUC |
| | V5:P2A::Blasti- | CCCAACCCCCUGCUGGGCCUGGACAGCACGGGCAG |
| | [STOP]-ARTi | CGGCGCCACCAACUUCAGCCUGCUGAAGCAGGCCG |
| | target sites | GCGACGUGGAGGAGAACCCCGGCCCCAUGAAGACC |
| | (RNA) | UUCAACAUCUCUCAGCAGGACCUUGAGCUGGUGGA |
| | | AGUUGCCACUGAGAAAAUCACCAUGCUCUAUGAGG |
| | | ACAACAAGCACCAUGUUGGUGCUGCCAUCAGGACC |
| | | AAGACAGGAGAAAUCAUCUCUGCUGUCCACAUUGAA |
| | | GCCUACAUUGGCAGGGUCACUGUGUGUGCAGAGGC |
| | | CAUUGCCAUUGGGUCUGCAGUCUCCAAUGGGCAGA |
| | | AGGACUUUGACACCAUUGUGGCUGUGAGGCACCCC |
| | | UACAGUGAUGAGGUGGACAGGUCCAUCAGAGUGGU |
| | | GUCCCCCUGUGGCAUGUGCAGGGAGCUCAUCUCAG |
| | | ACUAUGCCCCUGAUUGCUUUGUUCUGAUUGAGAUG |
| | | AAUGGCAAGCUGGUCAAGACAACCAUUGAGGAGCU |
| | | GAUUCCACUGAAAUACACCAGAAACUGA[STOP]UCC |
| | | GGAUGAUAUUGUAUACGAAUCCGGAUGAUGUUUUA |
| | | AUCGAAUCCGGAUGAUAUUGUUAUCGAA |
| SEQ ID | dsRed::Linker:: | ATGGCAAGCTCAGAAAATGTCATCACCGAGTTCATGC |
| NO: 55 | KRASG12R- | GATTCAAAGTGCGCATGGAGGGCACCGTGAACGGCC |
| | [STOP]-ARTi | ACGAGTTCGAGATCGAGGGAGAGGGAGAGGGCCGG |
| | target sites | CCATATGAGGGCCACAATACCGTGAAGCTGAAGGTG |
| | (DNA) | ACAAAGGGAGGACCACTGCCTTTCGCCTGGGACATC |
| | | CTGTCTCCTCAGTTTCAGTATGGCAGCAAGGTGTACG |
| | | TGAAGCACCCAGCCGACATCCCCGATTACAAGAAGC |
| | | TGTCCTTCCCAGAGGGCTTTAAGTGGGAGAGAGTGA |
| | | TGAACTTCGAGGACGGAGGAGTGGCAACCGTGACAC |
| | | AGGACAGCTCCCTGCAGGATGGCTGCTTTATCTATAA |
| | | GGTGAAGTTCATCGGCGTGAATTTTCCTAGCGATGG |
| | | CCCAGTGATGCAGAAGAAGACCATGGGCTGGGAGG |
| | | CCTCCACAGAGAGACTGTACCCTAGGGACGGCGTGC |
| | | TGAAGGGCGAGACACACAAGGCCCTGAAGCTGAAG |
| | | GATGGCGGCCACTATCTGGTGGAGTTTAAGAGCATC |
| | | TACATGGCCAAGAAGCCCGTGCAGCTGCCTGGCTAC |
| | | TATTACGTGGACGCCAAGCTGGATATCACCTCCCACA |
| | | ACGAGGATTATACAATCGTGGAGCAGTACGAGCGCA |
| | | CCGAGGGCCGGCACCACCTGTTCCTGGGGGGGGC |
| | | GGCTCTGGAGGAGGAGGAAGCGGAGGAGGAGGATC |
| | | CGGCATGACAGAGTACAAGCTGGTGGTGGTGGGAG |
| | | CCAGAGGCGTGGGCAAGTCCGCCCTGACCATCCAG |
| | | CTGATCCAGAATCACTTTGTGGACGAGTATGATCCCA |
| | | CAATCGAGGACTCTTACAGGAAGCAGGTGGTCATCG |
| | | ATGGCGAGACATGCCTGCTGGACATCCTGGATACAG |
| | | CCGGCCAGGAGGAGTATTCTGCCATGCGGGACCAGT |
| | | ACATGAGGACCGGCGAGGGCTTCCTGTGCGTGTTCG |
| | | CCATCAACAATACAAAGAGCTTCGAGGATATCCACCA |
| | | CTATCGCGAGCAGATCAAGCGGGTGAAGGACTCTGA |
| | | GGATGTGCCAATGGTGCTGGTGGGCAACAAGTGTGA |
| | | CCTGCCCAGCAGAACCGTGGACACAAAGCAGGCCCA |
| | | GGATCTGGCCAGGTCCTACGGCATCCCTTTCATCGA |
| | | GACAAGCGCCAAGACACGGCAGAGAGTGGAGGATG |
| | | CCTTTTATACCCTGGTGAGGGAGATCCGCCAGTACC |
| | | GGCTGAAGAAAATCTCAAAGGAAGAAAAGACCCCTG |
| | | GCTGCGTGAAAATTAAGAAATGTATTATTATGTGA[STOP] |
| | | TCCGGATGATATTGTATACGAATCCGGATGATGTT |
| | | TTAATCGAATCCGGATGATATTGTTATCGAACTCGAG |
| SEQ ID | dsRed::Linker:: | AUGGCAAGCUCAGAAAAUGUCAUCACCGAGUUCAU |
| NO: 56 | KRASG12R- | GCGAUUCAAAGUGCGCAUGGAGGGCACCGUGAACG |
| | [STOP]-ARTi | GCCACGAGUUCGAGAUCGAGGGAGAGGGAGAGGG |
| | target sites | CCGGCCAUAUGAGGGCCACAAUACCGUGAAGCUGA |
| | (RNA) | AGGUGACAAAGGGAGGACCACUGCCUUUCGCCUGG |
| | | GACAUCCUGUCUCCUCAGUUUCAGUAUGGCAGCAA |
| | | GGUGUACGUGAAGCACCCAGCCGACAUCCCCGAUU |
| | | ACAAGAAGCUGUCCUUCCCAGAGGGCUUUAAGUGG |
| | | GAGAGAGUGAUGAACUUCGAGGACGGAGGAGUGGC |
| | | AACCGUGACACAGGACAGCUCCCUGCAGGAUGGCU |
| | | GCUUUAUCUAUAAGGUGAAGUUCAUCGGCGUGAAU |
| | | UUUCCUAGCGAUGGCCCAGUGAUGCAGAAGAAGAC |
| | | CAUGGGCUGGGAGGCCUCCACAGAGAGACUGUACC |
| | | CUAGGGACGGCGUGCUGAAGGGCGAGACACACAAG |
| | | GCCCUGAAGCUGAAGGAUGGCGGCCACUAUCUGGU |
| | | GGAGUUUAAGAGCAUCUACAUGGCCAAGAAGCCCG |
| | | UGCAGCUGCCUGGCUACUAUUACGUGGACGCCAAG |
| | | CUGGAUAUCACCUCCCACAACGAGGAUUAUACAAUC |
| | | GUGGAGCAGUACGAGCGCACCGAGGGCCGGCACCA |
| | | CCUGUUCCUGGGCGGCGGCGGCUCUGGAGGAGGA |
| | | GGAAGCGGAGGAGGAGGAUCCGGCAUGACAGAGUA |
| | | CAAGCUGGUGGUGGUGGGAGCCAGAGGCGUGGGC |
| | | AAGUCCGCCCUGACCAUCCAGCUGAUCCAGAAUCA |
| | | CUUUGUGGACGAGUAUGAUCCCACAAUCGAGGACU |
| | | CUUACAGGAAGCAGGUGGUCAUCGAUGGCGAGACA |
| | | UGCCUGCUGGACAUCCUGGAUACAGCCGGCCAGGA |
| | | GGAGUAUUCUGCCAUGCGGGACCAGUACAUGAGGA |
| | | CCGGCGAGGGCUUCCUGUGCGUGUUCGCCAUCAAC |
| | | AAUACAAAGAGCUUCGAGGAUAUCCACCACUAUCGC |
| | | GAGCAGAUCAAGCGGGUGAAGGACUCUGAGGAUGU |
| | | GCCAAUGGUGCUGGUGGGCAACAAGUGUGACCUGC |
| | | CCAGCAGAACCGUGGACACAAAGCAGGCCCAGGAU |
| | | CUGGCCAGGUCCUACGGCAUCCCUUUCAUCGAGAC |
| | | AAGCGCCAAGACACGGCAGAGAGUGGAGGAUGCCU |
| | | UUUAUACCCUGGUGAGGGAGAUCCGCCAGUACCGG |
| | | CUGAAGAAAAUCUCAAAGGAAGAAAAGACCCCUGGC |
| | | UGCGUGAAAAUUAAGAAAUGUAUUAUUAUGUGA[STOP] |
| | | UCCGGAUGAUAUUGUAUACGAAUCCGGAUGAUG |
| | | UUUUAAUCGAAUCCGGAUGAUAUUGUUAUCGAACU |
| | | CGAG |
| SEQ ID | MYC 1891 | TGCTGTTGACAGTGAGCGCACGACGAGAACAGTTGA |
| NO: 59 | 97 mer (DNA) | AACATAGTGAAGCCACAGATGTATGTTTCAACTGTTC |
| | | TCGTCGTTTGCCTACTGCCTCGGA |
| SEQ ID | MYC 1891 | UGCUGUUGACAGUGAGCGCACGACGAGAACAGUUG |
| NO: 60 | 97 mer (RNA) | AAACAUAGUGAAGCCACAGAUGUAUGUUUCAACUGU |
| | | UCUCGUCGUUUGCCUACUGCCUCGGA |
| SEQ ID | Ren 660 | TGCTGTTGACAGTGAGCGACTCGTGAAATCCCGTTA |
| NO: 61 | 97 mer (DNA) | GTAATAGTGAAGCCACAGATGTATTACTAACGGGATT |
| | | TCACGAGGTGCCTACTGCCTCGGA |
| SEQ ID | Ren 660 | UGCUGUUGACAGUGAGCGACUCGUGAAAUCCCGUU |
| NO: 62 | 97 mer (RNA) | AGUAAUAGUGAAGCCACAGAUGUAUUACUAACGGG |
| | | AUUUCACGAGGUGCCUACUGCCUCGGA |
| SEQ ID | Ren 713 | TGCTGTTGACAGTGAGCGCAGGAATTATAATGCTTAT |
| NO: 63 | 97 mer (DNA) | CTATAGTGAAGCCACAGATGTATAGATAAGCATTATA |
| | | ATTCCTATGCCTACTGCCTCGGA |
| SEQ ID | Ren 713 | UGCUGUUGACAGUGAGCGCAGGAAUUAUAAUGCUU |
| NO: 64 | 97 mer (RNA) | AUCUAUAGUGAAGCCACAGAUGUAUAGAUAAGCAUU |
| | | AUAAUUCCUAUGCCUACUGCCUCGGA |
| SEQ ID | PTEN 1523 | TGCTGTTGACAGTGAGCGACCAGCTAAAGGTGAAGA |
| NO: 65 | 97 mer (DNA) | TATATAGTGAAGCCACAGATGTATATATCTTCACCTTT |
| | | AGCTGGCTGCCTACTGCCTCGGA |
| SEQ ID | PTEN 1523 | UGCUGUUGACAGUGAGCGACCAGCUAAAGGUGAAG |
| NO: 66 | 97 mer (RNA) | AUAUAUAGUGAAGCCACAGAUGUAUAUAUCUUCACC |
| | | UUUAGCUGGCUGCCUACUGCCUCGGA |
| SEQ ID | PTEN 1524 | TGCTGTTGACAGTGAGCGACAGCTAAAGGTGAAGAT |
| NO: 67 | 97 mer (DNA) | ATATTAGTGAAGCCACAGATGTAATATATCTTCACCTT |
| | | TAGCTGGTGCCTACTGCCTCGGA |
| SEQ ID | PTEN 1524 | UGCUGUUGACAGUGAGCGACAGCUAAAGGUGAAGA |
| NO: 68 | 97 mer (RNA) | UAUAUUAGUGAAGCCACAGAUGUAAUAUAUCUUCAC |
| | | CUUUAGCUGGUGCCUACUGCCUCGGA |

### CITATION LIST

### Patent Literature

EP2267131B1: Fire, A. et al. (2017). Genetic inhibition by double-stranded RNA.
EP1309726B2: Tuschl et al. (2018). RNA sequence-specific mediators of RNA interference.
EP1407044B2: Tuschl et al. (2017). RNA interference mediating small RNA molecules.
EP1546174B1: Bernstein, E. et al. (2011). Methods and compositions for RNA interference.
WO2014117050A2: Fellmann, C. et al. (2014). Modified miRNA as a scafold for shRNA

### Non-Patent Literature

1. Mohr, S. E., Smith, J. A., Shamu, C. E., Neumuller, R. A. & Perrimon, N. RNAi screening comes of age: improved techniques and complementary approaches. Nat Rev Mol Cell Bio 15, 591-600 (2014).
2. Housden, B. E. et al. Loss-of-function genetic tools for animal models: cross-species and cross-platform differences. Nat Rev Genet 18, 24-40 (2017).
3. Jackson, A. L. et al. Expression profiling reveals off-target gene regulation by RNAi. Nat Biotechnol 21, 635-637 (2003).
4. Scacheri, P. C. et al. Short interfering RNAs can induce unexpected and divergent changes in the levels of untargeted proteins in mammalian cells. P Natl Acad Sci Usa 101, 1892-1897 (2004).
5. Lin, X. et al. siRNA-mediated off-target gene silencing triggered by a 7 nt complementation. Nucleic Acids Res 33, 4527-4535 (2005).
6. Yesbolatova, A. et al. The auxin-inducible degron 2 technology provides sharp degradation control in yeast, mammalian cells, and mice. Nat Commun 11, 5701 (2020).
7. Vert, J.-P., Foveau, N., Lajaunie, C. & Vandenbrouck, Y. An accurate and interpretable model for siRNA efficacy prediction. Bmc Bioinformatics 7, 520 (2006).
8. Fellmann, C. et al. Functional Identification of Optimized RNAi Triggers Using a Massively Parallel Sensor Assay. Mol Cell 41, 733-746 (2011).
9. Schwarz, D. S. et al. Asymmetry in the Assembly of the RNAi Enzyme Complex. Cell 115,199-208 (2003).
10. Neumann, T. et al. Quantification of experimentally induced nucleotide conversions in high-throughput sequencing datasets. Bmc Bioinformatics 20, 258 (2019).
11. Liao, Y., Smyth, G. K. & Shi, W. featureCounts: an efficient general purpose program for assigning sequence reads to genomic features. Bioinformatics 30, 923-930 (2014).
13. Neumuller, R. A. et al. Stringent Analysis of Gene Function and Protein-Protein Interactions Using Fluorescently Tagged Genes. Genetics 190, 931-940 (2012).
14. Wissel, S. et al. A Combination of CRISPR/Cas9 and Standardized RNAi as a Versatile Platform for the Characterization of Gene Function. G3 Genes Genomes Genetics 6, 2467-2478 (2016).
15. Fellmann, C. et al. An Optimized microRNA Backbone for Effective Single-Copy RNAi. Cell Reports 5, 1704-1713 (2013).
15. Pelossof, R. et al. Prediction of potent shRNAs with a sequential classification algorithm. Nature Biotechnology 35(4), 350-353 (2017).

1. A method of suppressing gene expression, comprising
providing a mammalian cell with a suppression target RNA molecule comprising a heterologous RNAi target site operatively linked to a RNA sequence from a gene of interest, or with a DNA encoding said suppression target RNA molecule;
providing to the cell an inhibitory RNA that has a complementary nucleotide sequence to the heterologous RNAi target site;
wherein binding of the inhibitory RNA to the heterologous RNAi target site suppresses gene expression of the operatively linked RNA sequence from the gene of interest;
and wherein said heterologous RNAi target site has a size of at least 18 nucleotides in length.

2. The method of claim 1, wherein the DNA encoding said suppression target RNA molecule is an expression vector or a genomic DNA of the mammalian cell; and/or wherein the inhibitory RNA is expressed from a DNA encoding said inhibitory RNA in said mammalian cell, wherein expression of the inhibitory RNA is optionally inducible expression by an inducible promoter, or the DNA encoding said inhibitory RNA comprises a tetracycline-responsive element promoter.

3. The method of claim 1, wherein the inhibitory RNA is selected from shRNA, siRNA, miRNA or a combination thereof, wherein the shRNA is optionally shRNAmir; and/or wherein the inhibitory RNA comprises a double strand, wherein one strand of the double strand is complementary to the heterologous RNAi target site.

4. The method of claim 1, wherein the heterologous RNAi target site is a non-murine and/or non-human, optionally a non-mammalian nucleic acid sequence.

5. The method of to claim 1, wherein the complementary nucleotide sequence of the inhibitory RNA comprises or consists of the nucleic acid sequence 5′-UUCGWWWNNAHHWWCAUCCGGN-3′ (SEQ ID NO: 2), wherein W is A or U, H is A or U or C, and N is A or U or G or C; wherein the complementary nucleotide sequence of the inhibitory RNA optionally comprises or consists of a nucleic acid sequence selected from any one of SEQ ID NO: 4, 6, 8, 10, 12, 14, 16, 18, 20, 22, 24, or 26.

6. The method of claim 1, wherein the heterologous RNAi target site comprises or consists of the nucleic acid sequence 5′-NCCGGAUGWWDDUNNWWWCGAA-3′, (SEQ ID NO: 28), wherein W is U or A, D is U or A or G, and N is U or A or C or G; wherein the heterologous RNAi target site optionally comprises or consists of a nucleic acid sequence selected from any one of SEQ ID NO: 30, 32, 34, 36, 38, or 40.

7. The method of claim 1, wherein the mammalian cell lacks a functional genetic copy of the gene of interest in its genome apart from a DNA encoding said suppression target RNA molecule.

8. The method of claim 1, wherein the gene of interest is an exogenous gene; wherein an endogenous variant of the exogenous gene in the genome of the mammalian cell is optionally disrupted.

9. The method of claim 1, wherein the gene of interest is EGFR, KRAS or STAG1.

10. The method of claim 1, wherein the gene of interest is an endogenous gene, optionally the endogenous gene is an oncogene or tumor suppressor gene, wherein the oncogene or tumor suppressor gene is optionally EGFR, KRAS or STAG1.

11. A nucleic acid comprising the nucleic acid sequence 5′-TTCGWWWNNAHHWWCATCCGGN-3′ (SEQ ID NO: 1), wherein W is A or T, H is A or T or C, and N is A or T or G or C; wherein A is adenine, C is cytosine, G is guanine, T is thymine in DNA or uracil in RNA; the nucleic acid is optionally RNA comprising the nucleic acid sequence 5′-UUCGWWWNNAHHWWCAUCCGGN-3′ (SEQ ID NO: 2); and/or the nucleic acid sequence is optionally selected from any one of SEQ ID NO: 3, 5, 7, 9, 11, 13, 15, 17, 19, 21, 23, 25.

12. The nucleic acid of claim 11, wherein the sequence of SEQ ID NO: 1 is double stranded.

13. The nucleic acid of claim 11, wherein the nucleic acid is RNA and comprises a stem-loop structure, or wherein the nucleic acid is DNA and encodes or transcribes a RNA that comprises a stem-loop structure; and/or wherein the nucleic acid is RNA and is selected from shRNA, siRNA, miRNA or a combination thereof, wherein the shRNA is optionally shRNAmir, or wherein the nucleic acid is DNA and encodes or transcribes a RNA that is a shRNA, a siRNA, a miRNA or a combination thereof, wherein the shRNA is optionally a shRNAmir.

14. A nucleic acid comprising the nucleic acid sequence 5′-NCCGGATGWWDDTNNWWWCGAA-3′ (SEQ ID NO: 27), wherein W is T or A, D is T or A or G, and N is T or A or C or G, wherein the nucleic acid optionally comprises or consists of a nucleic acid sequence selected from any one of SEQ ID NO: 29, 31, 33, 35, 37, 39, 41, 43, 45, 47, 49, 51, 53, 55, 57.

15. A nucleic acid comprising a promoter, an expression sequence and a nucleic acid sequence of a nucleic acid according to claim 11; wherein the sequence of a nucleic acid according to claim 11 is optionally located in a 5′ UTR of the expression sequence, in the expression sequence or in a 3′ UTR of the expression sequence; and/or wherein the expression sequence is optionally a protein coding sequence, more preferably an exon.

16. A vector comprising a nucleic acid of claim 11 as DNA.

17. A mammalian cell comprising a DNA encoding a gene of interest operatively linked to a sequence of a heterologous RNAi target site;
further comprising a DNA encoding an inhibitory RNA that has a complementary region to the heterologous RNAi target site;
wherein said heterologous RNAi target site has a size of at least 18 nucleotides in length;
wherein the gene of interest is optionally on an exogenous gene;
wherein an endogenous variant of the exogenous gene is optionally disrupted.

18. A mammalian cell comprising a nucleic acid of claim 11.

19. A method of determining the effects of a loss of function of a gene of interest in a mammalian cell, comprising
providing a mammalian cell with a DNA that transcribes a suppression target RNA molecule comprising a heterologous RNAi target site operatively linked to a RNA sequence from the gene of interest;
providing to the cell an inhibitory RNA that has a complementary nucleotide sequence to the heterologous RNAi target site;
wherein binding of the inhibitory RNA to the heterologous RNAi target site suppresses gene expression of the operatively linked RNA sequence from the gene of interest;
and wherein said heterologous RNAi target site has a size of at least 18 nucleotides in length;
and observing a change of phenotype of the mammalian cell between the mammalian cells with the provided inhibitory RNA and without providing the inhibitory RNA; wherein providing to the cell an inhibitory RNA optionally comprises suppressing gene expression according to claim 1.

20. A nucleic acid comprising a promoter, an expression sequence and a nucleic acid sequence of a nucleic acid according to claim 14; wherein the sequence of a nucleic acid according to claim 14 is optionally located in a 5′ UTR of the expression sequence, in the expression sequence or in a 3′ UTR of the expression sequence; and/or wherein the expression sequence is optionally a protein coding sequence, more preferably an exon.

21. A vector comprising a nucleic acid of claim 14 as DNA.

22. A mammalian cell comprising a nucleic acid of claim 14.
